(19) United States
(12) Patent Application Publication
(10) Pub. No.: US 2025/0250573 A1
(43) Pub. Date: Aug. 7, 2025

(54) POLYNUCLEIC ACID MOLECULES FOR INHIBITING EXPRESSION OF FXI, PHARMACEUTICAL COMPOSITIONS, AND USES THEREOF

(71) Applicant: Sirius Therapeutics, Inc., San Diego, CA (US)

(72) Inventors: Curt BRADSHAW, San Diego, CA (US); Anthony NICHOLAS, Oregon, WI (US)

(21) Appl. No.: 19/088,429

(22) Filed: Mar. 24, 2025

Related U.S. Application Data
(63) Continuation of application No. PCT/US2023/075046, filed on Sep. 25, 2023.
(60) Provisional application No. 63/410,084, filed on Sep. 26, 2022.

(51) Int. Cl. C12N 15/113; A61P 7/02

(52) U.S. Cl. CPC C12N 15/1137; A61P 7/02; C12N 2310/11; C12N 2310/14; C12N 2310/315; C12N 2310/321; C12N 2310/322; C12N 2310/333; C12N 2310/335; C12N 2310/351; C12Y 304/21027

(57) ABSTRACT

Disclosed herein are polynucleic acid molecules, pharmaceutical compositions, and methods for suppressing the expression of coagulation factor XI (FXI) gene.

### CROSS-REFERENCE

This application is a continuation of International Application No. PCT/US2023/075046, filed Sep. 25, 2023, which claims the benefit of U.S. Provisional Application No. 63/410,084, filed on Sep. 26, 2022, all of which are incorporated herein by reference in their entirety.

### SEQUENCE LISTING

The instant application contains a Sequence Listing which has been submitted electronically in XML format and is hereby incorporated by reference in its entirety. Said XML copy, created on Mar. 21, 2025, is named 61382-714.301_SL.xml and is 893,891 bytes in size.

### BACKGROUND OF THE DISCLOSURE

The discovery of RNA interference (RNAi) as a cellular mechanism that selectively degrades mRNAs allows for both the targeted manipulation of cellular phenotypes in cell culture and the potential for development of directed therapeutics (Behlke, 2006, Mol. Ther. 13, 644-670; Xie et al., 2006, Drug Discov. Today 11, 67-73).

Thrombosis is the formation of a blood clot, known as a thrombus, which occurs within a blood vessel. This can prevent blood from flowing normally via the circulatory system and can be deadly. Coagulation Factor XI (FXI or F11) is a protein encoded by the FXI gene. FXI plays an important role in the coagulation cascade and is made primarily by cells in the liver. Accordingly, there is a need for developing an effective FXI inhibitor without cytotoxicity. The polynucleic acid molecules, conjugates thereof, and methods described herein satisfy this need and provide related advantages.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### SUMMARY OF THE DISCLOSURE

To meet the need for a more effective FXI inhibitor, provided herein, in one aspect, includes a polynucleic acid molecule for modulating expression of coagulation factor XI (FXI) gene, wherein the polynucleic acid molecule comprises a nucleic acid sequence that is at least 80%, at least 85%, at least 90% identical to a nucleic acid sequence in Tables 1-2 and Tables 7-9. In some instances, wherein the polynucleic acid molecule is a double-stranded nucleic acid molecule comprising a sense strand (passenger strand) and an antisense strand (guide strand). In some instances, the sense strand comprises a nucleic acid sequence that is at least 80%, at least 85%, at least 90%, at least 95% identical to a nucleic acid sequence selected from SEQ ID NOs: 101-150 and 220. In some instances, the antisense strand comprises a nucleic acid sequence that is at least 80%, at least 85%, at least 90%, at least 95% identical to a nucleic acid sequence selected from SEQ ID NOs: 1-50 and 215-216.

In some instances, the sense strand comprises a nucleic acid sequence comprising at least 14, 15, 16, 17, 18, 19, or 20 consecutive sequences of a nucleic acid sequence selected from SEQ ID NOs: 101-150 and 220 with no more than 1, 2, 3, or 4 mismatches. In some instances, the antisense strand comprises a nucleic acid sequence comprising at least 14, 15, 16, 17, 18, 19, 20, 21, or 22 consecutive sequences of a nucleic acid sequence selected from SEQ ID NOs:1-50 and 215-216 with no more than 1, 2, 3, or 4 mismatches. In some instances, the sense strand comprises a nucleic acid sequence of SEQ ID NOs: 101-150 and 220 and the antisense strand comprises a nucleic acid sequence of SEQ ID NOs: 1-50 and 215-216. In some instances, the sense strand comprises a nucleic acid sequence that is at least 90%, at least 95% identical to a nucleic acid sequence selected from SEQ ID NOs: 104, 106-107, 109-111, 113-114, 135, 150, and 220. In some instances, the antisense strand comprises a nucleic acid sequence that is at least 90%, at least 95% identical to a nucleic acid sequence selected from SEQ ID NOs: 4, 6-7, 9-11, 13-14, 35, 50, and 215-216. In some instances, the sense strand comprises a nucleic acid sequence selected from SEQ ID NOs: 104, 106-107, 109-111, 113-114, 135, 150, and 220 and the antisense strand comprises a nucleic acid sequence selected from a nucleic acid sequence of SEQ ID NOs: 4, 6-7, 9-11, 13-14, 35, 50, and 215-216.

In some instances, the antisense strand comprises 5′-nNfnnnNfnNfNfnnnnNfnNfnnnnnnn-3′, 5′-nNfnnnNfnnnnnnnNfnNfnnnnnnn-3′, 5′-nNfnnnnNfnnnnNfnNfnnnnnnnnn-3′, 5′-nNfnnnnNfnnnnNfnNfnNfnnnnnnn-3′, or 5′-nNfnnnnnnnnnNfnNfnNfnnnnnnn-3′, wherein “Nf” stands for a 2′-fluoro modified nucleotide, and wherein “n” stands for a 2′-O-methyl modified nucleotide. In some instances, the sense strand comprises 5′-nnnnnnNfnNfnNfnnnnnnnnnn-3′, 5′-nnnnnnNfnNfNfNfnnnnnnnnnn-3′, 5′-nnnnnnnnNfNfNfnnnnnnnnnn-3′, or 5′-nnnnnnNfnNfnNfnnnnnnnnnn-invdN-invdN-3′ wherein “Nf” stands for a 2′-fluoro modified nucleotide, and wherein “n” stands for a 2′-O-methyl modified nucleotide, and wherein “invdN” stands for an inverted deoxy-nucleotide. In some instances, the sense strand comprises 5′-nnnnnnNfnNfnNfnnnnnnnnnn-3′, wherein the antisense strand comprises 5′-nNfnnnnnnnnnNfnNfnNfnnnnnnn-3′, wherein “Nf” stands for a 2′-fluoro modified nucleotide, and wherein “n” stands for a 2′-O-methyl modified nucleotide. In some instances, the sense strand comprises 5′-nnnnnnNfnNfnNfnnnnnnnnnn-3′, wherein the antisense strand comprises 5′ _nNfnnnnNfnnnnNfnNfnNfnnnnnnn-3′, wherein “Nf” stands for a 2′-fluoro modified nucleotide, and wherein “n” stands for a 2′-O-methyl modified nucleotide.

In some instances, the polynucleic acid molecule comprises a modified internucleotide linkage, optionally wherein the modified internucleotide linkage is a phosphorothioate internucleotide linkage. In some instances, the modified internucleotide linkage comprises a stereochemically enriched phosphorothioate internucleotide linkage. In some instances, the modified internucleotide linkage is an SP chiral internucleotide phosphorothioate linkage. In some instances, the polynucleic acid comprises a plurality of modified internucleotide linkages, and at least 1, 2, 3, or 4 of the plurality of modified internucleotide linkages are stereochemically enriched phosphorothioate internucleotide linkages. In some instances, the stereochemically enriched phosphorothioate internucleotide linkages comprise both R- and S-isomers. In some instances, the stereochemically enriched phosphorothioate is disposed between two consecutive nucleosides that are two of six 5′ or 3′-terminal nucleosides of the sense strand or the antisense strand.

In some instances, the polynucleic acid molecule comprises a hypoxanthine nucleobase-containing nucleoside substitution. In some instances, the hypoxanthine nucleobase-containing nucleoside substitution is an inosine substitution, optionally wherein the inosine substitution comprises 2′-O-methylinosine-3′-phosphate. In some instances, the inosine substitution is within a seed region of the antisense strand. In some instances, the inosine substitution is within 7 nucleotides from the 5′ end of the antisense strand, optionally wherein the inosine substitution is in the first nucleotide from the 5′ end of the antisense strand.

In some instances, the first nucleotide from the 5′ end of the antisense strand is substituted by a uridine or an adenosine, optionally wherein the uridine comprises 2′-O-methyluridine-3′-phosphate, or optionally wherein the adenosine comprises 2′-O-methyl-8-bromo-adenosine-3′-phopshate.

In some instances, wherein the sense strand comprises a nucleic acid sequence that is at least 80%, at least 85%, at least 90%, at least 95% identical to a nucleic acid sequence selected from SEQ ID NOs: 151-200, 214, and 221. In some instances, the antisense strand comprises a nucleic acid sequence that is at least 80%, at least 85%, at least 90%, at least 95% identical to a nucleic acid sequence selected from SEQ ID NOs: 51-100, 201-213, and 217-219. In some instances, the sense strand comprises a nucleic acid sequence selected from a nucleic acid sequence of SEQ ID NOs: 151-200, 214, and 221 and the antisense strand comprises a nucleic acid sequence selected from a nucleic acid sequence of SEQ ID NOs: 51-100, 201-213, and 217-219.

In another aspects, the present disclosure includes a polynucleic acid molecule for modulating expression of coagulation factor XI (FXI) gene, wherein the polynucleic acid molecule comprises:
(a) an antisense strand comprising a nucleotide sequence of SEQ ID NO: 4, 6-7, 9-11, 13-14, 35, 50, and 215-216 and a sense strand comprising a nucleotide sequence of SEQ ID NO: 104, 106-107, 109-111, 113-114, 135, 150, and 220; or
(b) an antisense strand comprising a nucleotide sequence of SEQ ID NO: 54, 56-57, 59-61, 63-64, 85, 100, 201-213, and 217-219 and a sense strand comprising a nucleotide sequence of SEQ ID NO: 154, 156-157, 159-161, 163-164, 185, 200, 214, and 221.

In some instances, the polynucleic acid molecule comprise:
(a) an antisense strand comprising a nucleotide sequence of AUAAAUGUCUUUGUUGCAAGCGC (SEQ ID NO:11) and a sense strand comprising a nucleotide sequence of GCUUGCAACAAAGACAUUUAU (SEQ ID NO: 111);
(b) an antisense strand comprising a nucleotide sequence of AUGUCUUUGUUGCAAGCGCUUAU (SEQ ID NO: 9) and a sense strand comprising a nucleotide sequence of AAGCGCUUGCAACAAAGACAU (SEQ ID NO: 109);
(c) an antisense strand comprising a nucleotide sequence of AAUGUCUUUGUUGCAAGCGCUUA (SEQ ID NO: 10) and a sense strand comprising a nucleotide sequence of AGCGCUUGCAACAAAGACAUU (SEQ ID NO: 110);
(d) an antisense strand comprising a nucleotide sequence of UUAUAGUUUAUGCCCUUCAUGUC (SEQ ID NO: 13) and a sense strand comprising a nucleotide sequence of CAUGAAGGGCAUAAACUAUAA (SEQ ID NO: 113);
(e) an antisense strand comprising a nucleotide sequence of AUAGGUAAAAAACUGGCAGCGGA (SEQ ID NO: 35) and a sense strand comprising a nucleotide sequence of CGCUGCCAGUUUUUUACCUAU (SEQ ID NO: 135)
(f) an antisense strand comprising a nucleotide sequence of IUAAAUGUCUUUGUUGCAAGCGC (SEQ ID NO: 215) and a sense strand comprising a nucleotide sequence of GCUUGCAACAAAGACAUUUAU (SEQ ID NO: 111); or
(g) an antisense strand comprising a nucleotide sequence of UUAAAUGUCUUUGUUGCAAGCGC (SEQ ID NO: 216) and a sense strand comprising a nucleotide sequence of GCUUGCAACAAAGACAUUUAA (SEQ ID NO: 220).

In another aspect, the present disclosure includes a polynucleic acid molecule for modulating expression of coagulation factor XI (FXI) gene, wherein the polynucleic acid molecule comprises:
(a) an antisense strand comprising a nucleotide sequence of asUfsaaaugucuuUfgUfuGfcaagcsgsc (SEQ ID NO: 61) and a sense strand comprising a nucleotide sequence of gscsuugcAfaCfaAfagacauuuau (SEQ ID NO: 161);
(b) an antisense strand comprising a nucleotide sequence of asUfsaaaugucuuUfgUfuGfcsaagcsgsc (SEQ ID NO: 211) and a sense strand comprising a nucleotide sequence of gscsuugcAfaCfaAfagacauuuau (SEQ ID NO: 161);
(c) an antisense strand comprising a nucleotide sequence of asUfsaaaugucuuUfgUfsuGfcaagcsgsc (SEQ ID NO: 212) and a sense strand comprising a nucleotide sequence of gscsuugcAfaCfaAfagacauuuau (SEQ ID NO: 161);
(d) an antisense strand comprising a nucleotide sequence of asUfsaaaugucuuUfgUfuGfcaasgcsgsc (SEQ ID NO: 213) and a sense strand comprising a nucleotide sequence of gscsuugcAfaCfaAfagacauuuau (SEQ ID NO: 161);
(e) an antisense strand comprising a nucleotide sequence of asUfsaaaugucuuUfgUfuGfcaagcsgsc (SEQ ID NO: 61) and a sense strand comprising a nucleotide sequence of gscsuugcAfaCfaAfagacauuuau(invdT)(invdT) (SEQ ID NO: 214);
(f) an antisense strand comprising a nucleotide sequence of asUfsgucuUfuguuGfcAfaGfcgcuusasu (SEQ ID NO: 204) and a sense strand comprising a nucleotide sequence of asasgcgcUfuGfcAfacaaagacau (SEQ ID NO: 159);
(g) an antisense strand comprising a nucleotide sequence of asAfsugucUfuuguUfgCfaAfgcgcususa (SEQ ID NO: 205) and a sense strand comprising a nucleotide sequence of asgscgcuUfgCfaAfcaaagacauu (SEQ ID NO: 160);
(h) an antisense strand comprising a nucleotide sequence of asUfsaaauGfucuuUfgUfuGfcaagcsgsc (SEQ ID NO: 206) and a sense strand comprising a nucleotide sequence of gscsuugcAfaCfaAfagacauuuau (SEQ ID NO: 161);
(i) an antisense strand comprising a nucleotide sequence of usUfsauagUfuuauGfcCfcUfucaugsusc (SEQ CD NO: 207) and a sense strand comprising a nucleotide sequence of csasugaaGfgGfcAfuaaacuauaa (SEQ ID NO: 163).
(j) an antisense strand comprising a nucleotide sequence of asUfsagguAfaaaaAfcUfgGfcagcgsgsa (SEQ ID NO: 209) and a sense strand comprising a nucleotide sequence of csgscugcCfaGfuUfuuuuaccuau (SEQ ID NO: 185);
(k) an antisense strand comprising a nucleotide sequence of isUfsaaaugucuuUfgUfuGfcaagcsgsc (SEQ ID NO: 217) and a sense strand comprising a nucleotide sequence of gscsuugcAfaCfaAfagacauuuau (SEQ ID NO: 161);
(l) an antisense strand comprising a nucleotide sequence of usUfsaaauGfucuuUfgUfuGfcaagcsgsc (SEQ ID NO: 218) and a sense strand comprising a nucleotide sequence of gscsuugcAfaCfaAfagacauuuaa (SEQ ID NO: 222), or
(m) an antisense strand comprising a nucleotide sequence of a4sUfsaaauGfucuuUfgUfuGfcaagcsgsc (SEQ ID NO: 219) and a sense strand comprising a nucleotide sequence of gscsuugcAfaCfaAfagacauuuau (SEQ ID NO: 161);
wherein “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “invdT” refers to 3′ inverted thymidine, “i” refers to 2′-O-methylinosine-3′-phosphate, and “a4” refers to 2′-O-methyl-8-bromo-adenosine-3′-phopshate.

In another aspect, the present disclosure includes a polynucleic acid molecule conjugate for modulating expression of coagulation factor XI (FXI) gene, wherein the polynucleic acid molecule conjugate comprises a polynucleic acid molecule as disclosed herein and an asialoglycoprotein receptor targeting moiety.

In some instances the polynucleic acid molecule and the asialoglycoprotein receptor targeting moiety is coupled via a linker. In some instances, the linker comprises formula (IV) below,

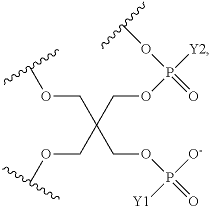

wherein at least one of Y1 and Y2 is a nucleotide in the polynucleic acid molecule. In some instances, the Y1 is the last nucleotide on the 3′-terminus of the sense strand of the polynucleic acid molecule, or wherein the Y2 is the first nucleotide on the 5′-terminus of the sense strand of the polynucleic acid molecule. In some instances, wherein the Y1 and Y2 are two consecutive nucleotides in the polynucleic acid molecule

In some instances, the asialoglycoprotein receptor targeting moiety comprises N-Acetylgalactosamine (GalNAc) or galactose.

In some instances, the linker and the asialoglycoprotein receptor targeting moiety with the last nucleotide on the 3′-terminus of the sense strand of the polynucleic acid molecule are shown in:

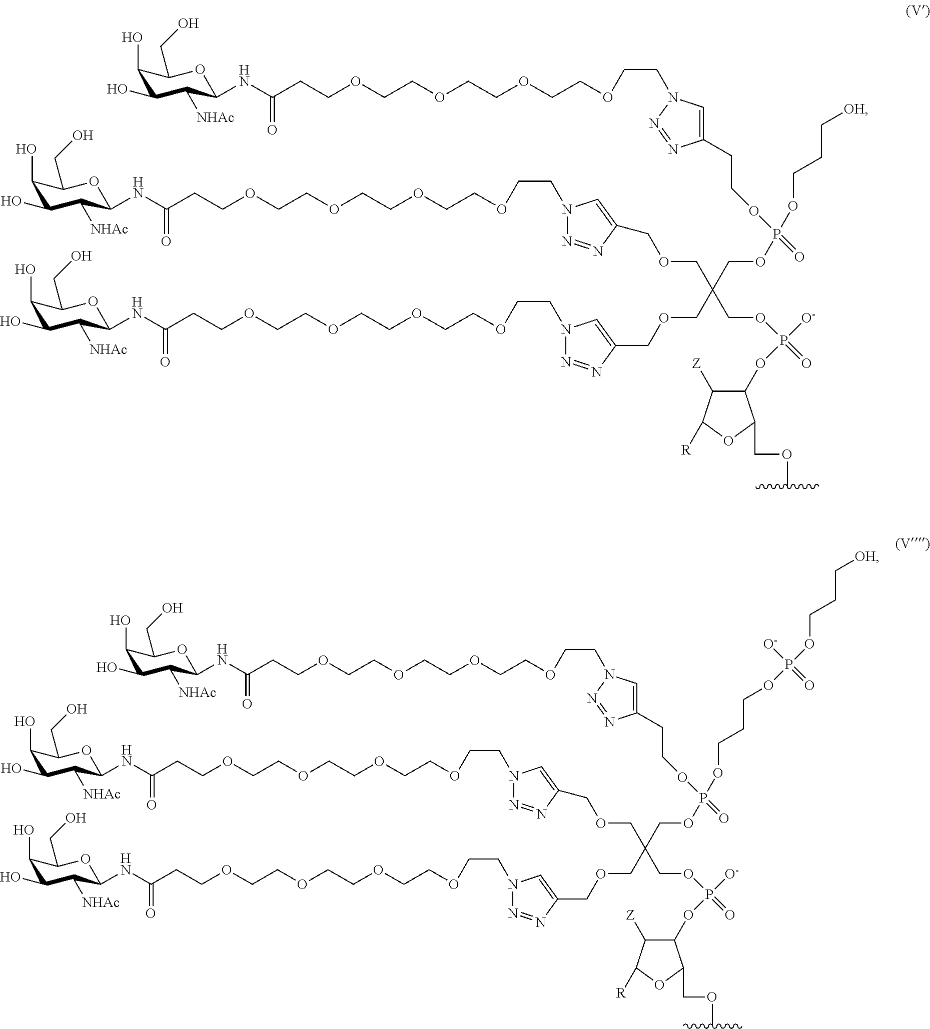

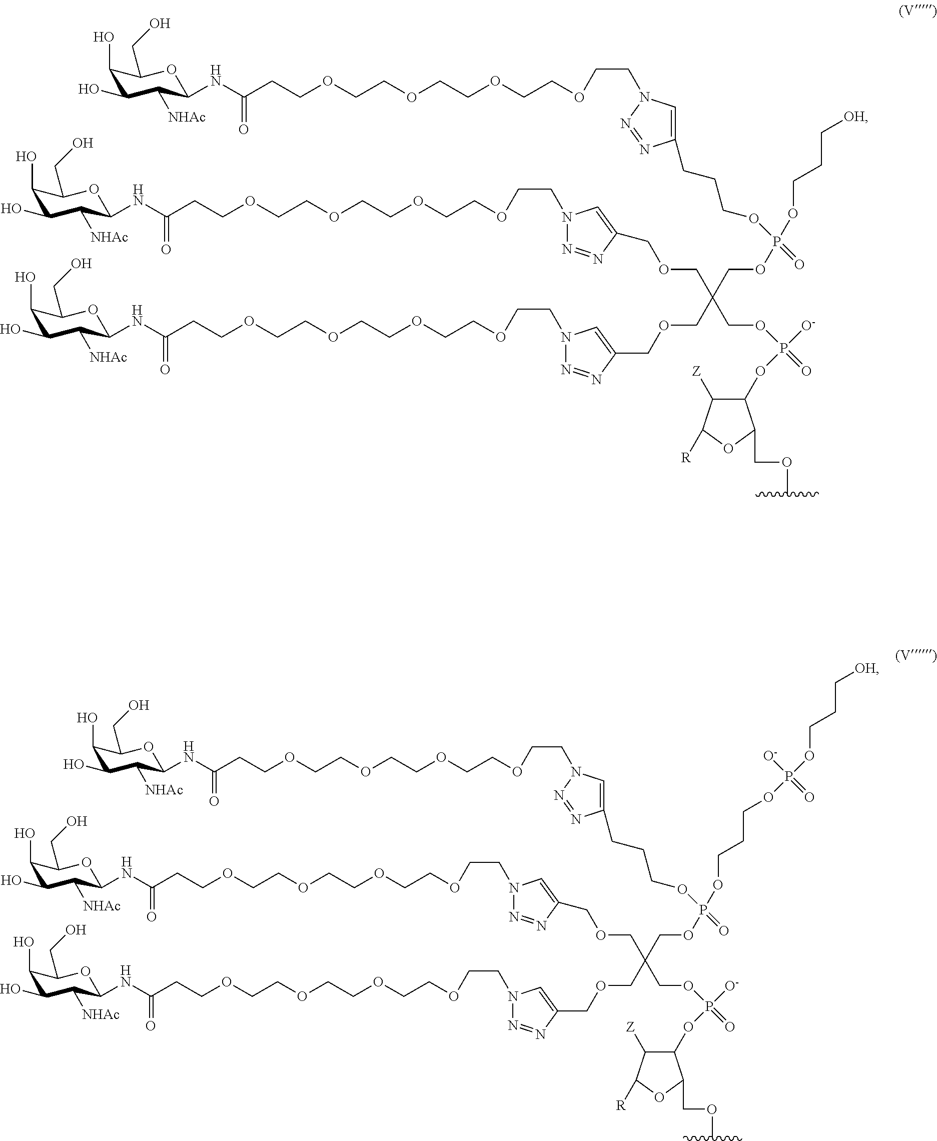

wherein Z in formula (V′), (V″″), (V′″″), or (V″″″) is —H, —OH, —O-Methyl, —F, or —O-methoxyethyl, and R in formula (V′) is adenine, uracil, guanine, cytosine, thymine, abasic, or others.

In another aspect, the present disclosure includes a pharmaceutical composition comprising a polynucleic acid molecule or a polynucleic acid molecule conjugate as disclosed herein, and a pharmaceutically acceptable excipient.

In another aspect, the present disclosure includes a method of modulating mRNA expression of coagulation factor XI (FX) gene in a subject, comprising: administering to the subject a polynucleic acid molecule, a polynucleic acid molecule conjugate, or a pharmaceutical composition as disclosed herein, thereby modulating the mRNA expression of FXI gene in the subject.

In another aspect, the present disclosure includes a method of modulating FXI or FXIa protein levels or FXI or FXIa activity in a subject in need thereof, comprising: administering to the subject a polynucleic acid molecule, a polynucleic acid molecule conjugate, or a pharmaceutical composition as disclosed herein, thereby modulating the FXI or FXIa protein levels or FXI or FXIa activity in the subject. In some instances, the subject in need thereof is diagnosed of, or suffers from thrombosis or a symptom thereof.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative aspects, in which the principles of the disclosure are utilized, and the accompanying drawings below.

FIG. 1 depicts the in vitro efficacy of siRNAs targeting FXI from SRS-000579 to SRS-000602 in primary human hepatocytes. Each siRNA was tested at three concentrations: 100 nM, 30 nM, and 10 nM. Three bars of each siRNA in the bar graph represent the average percentage of inhibition in 100 nM, 30 nM, and 10 nM, respectively from left to right. Three replicates were run per dose level per each siRNA. Error bars represent the standard deviation.

FIG. 2 depicts the in vitro efficacy of siRNAs targeting FXI from SRS-000603 to SRS-000628 in primary human hepatocytes. Each siRNA was tested at three concentrations: 100 nM, 30 nM, and 10 nM. Three bars of each siRNA in the bar graph represent the average percentage of inhibition in 100 nM, 30 nM, and 10 nM, respectively from left to right. Three replicates were run per dose level per each siRNA. Error bars represent the standard deviation.

FIG. 3 depicts the in vivo efficacy of SRS-000007 siRNA targeting FXI or Saline control in cynomolgus monkeys. SRS-000007 was tested at two dose levels, 1 mg/kg and 5 mg/kg via single subcutaneous injection. Results are shown in % change in plasma FXI protein compared to the pre-dose baseline.

FIG. 4 depicts dose response curves of SRS-000007 in three different donor lots of primary human hepatocytes.

FIG. 5 depicts % change in plasma FXI circulating protein levels relative to the pre-dose baseline in cynomolgus monkeys treated with siRNAs shown in Table 7.

FIG. 6 depicts % change in plasma FXI circulating protein levels relative to the pre-dose baseline in cynomolgus monkeys treated with siRNAs shown in Table 8.

FIG. 7 depicts dose response curves of SRS-000007 and SRS-002331 in two different donor lots of primary human hepatocytes.

### DETAILED DESCRIPTION OF THE DISCLOSURE

The gene of FXI is located on the distal end of the long arm of chromosome 4 (4q35.2). The mRNA of FXI (NM_000128.4) contains 3053 bp, which divides into 15 exons (exon 2 signal peptide, exons 3-10 apple domains, exons 11-15 protease domain). (see e.g., Mohammed et al., Thromb Res. 2018; 161:94-105 and NCBI Reference Sequence No: NM_000128.4).

Factor XI (FXI) is the zymogen of the coagulation protease factor XIa (FXIa) and is comprises of 625 amino acid residues that contains four apple domains (or PAN domains which stands for Plasminogen-Apple-Nematode; A1 to A4) and a trypsin-like catalytic domain. In some instances, FXI is also known as Plasma Thromboplastin Antecedent or PTA. Similar to other coagulation protease precursors, plasma FXI is synthesized primarily in hepatocytes. In some instances, in human, FXI is also expressed in the Islets of Langerhans in the pancreas, and in renal tubule cells of kidney. Each FXI subunit may be converted to its active form by thrombins (e.g., α-thrombin, β-thrombin, γ-thrombin, and meizothrombin) or Factor XIIa. In some cases, FXI can be autoactivated by FXIa in the presence of polyanions. Regardless of activating protease, FXI action requires cleavage of the Arg369-Ile370 bond to become active FXIa.

As such, targeting FXI can achieve an antithrombotic effect without causing severe bleeding, and further provide prevention or treatment of thrombosis.

Described herein is a polynucleic acid molecule for modulating expression of FXI gene. In some aspects, the polynucleic acid molecule is a single-stranded nucleic acid molecule. In some aspects, the polynucleic acid molecule is a double-stranded nucleic acid molecule that comprises a sense strand and an antisense strand. In some aspects, the polynucleic acid molecule comprises a nucleic acid sequence in Tables 1-2 and Tables 7-9. Accordingly, provided herein are various target regions of human FXI mRNA the polynucleic acid molecule described herein hybridizes to. In some cases, provided herein is the sequences of the polynucleic acid molecule described herein. In some cases, provided herein is the possible modifications of the polynucleic acid molecule described herein. In some cases, provided herein is the possible conjugates of the polynucleic acid molecule described herein.

Also described herein is a method of modulating expression of FXI mRNA or protein in a subject. Described further herein is a method of modulating FXI or FXIa activity levels in a subject in need thereof.

### Definitions

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed with in the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

“Percent (%) sequence identity” or “Percent (%) identity” with respect to the nucleic acid sequences identified herein is defined as the percentage of nucleic acid in a candidate sequence that are identical with the nucleic acid sequence being compared, after aligning the sequences considering any conservative substitutions as part of the sequence identity.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and the like. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Unless defined otherwise, all technical and scientific terms used herein have the same meaning as commonly understood by one of ordinary skill in the art to which the polynucleic acid molecules, the polynucleic acid molecule conjugates, the pharmaceutical compositions, the methods and other aspects belong.

As used herein, the term “complementary” indicates a sufficient degree of complementarity between two nucleic acid molecules that bind stably and specifically to avoid nonspecific binding.

As used herein, the term “polynucleic acid” and the term “poly nucleotide” are interchangeably used to refer a chain of nucleotides. The term “nucleotide” includes a sequence “G,” “C,” “A,” “T” and “U” each generally stand for a nucleotide that contains guanine, cytosine, adenine, thymidine and uracil as a base. In some instances, the “nucleotide” can refer to a modified nucleotide (e.g., with modified sugar moiety, modified base, modified internucleotide linkage, or combination thereof, including, but not limited to 2′-modified nucleotide, LNA, ENA, BNA, UNA, GNA etc.) In some instances, the “nucleotide” can refer to a modified nucleotide with a non-canonical base (e.g. including, but not limited to, 2-thiouridine, 2-thiothymidine, inosine, 2-aminopurine, 2,6-diaminopurine, dihydrouridine, 4-thiouridine, 4-thiothymidine, 2-thiocytidine).

As used herein, a “subject” can be any mammal, including a human and a non-human primate.

The term “condition,” as used herein, includes diseases, disorders, and susceptibilities. In some cases, the condition is an FXI related disorder or symptoms thereof.

As used herein, the term “treat,” “treating” or “treatment” of any disease or disorder refers, in one instance, to ameliorating the disease or disorder (i.e., slowing or arresting or reducing the development of the disease or at least one of the clinical symptoms thereof). In another instance, “treat”, “treating” or “treatment” refers to alleviating or ameliorating at least one physical parameter including those which may not be discernible by the patient. In yet another instance, “treat”, “treating” or “treatment” refers to modulating the disease or disorder, either physically, (e.g., stabilization of a discernible symptom), physiologically, (e.g., stabilization of a physical parameter), or both.

The terms “prevent,” “preventing,” and “prevention,” as used herein, refer to a decrease in the occurrence of pathology of a condition in a subject, who does not have, but is at risk of or susceptible to developing a disease or condition. The prevention may be complete, e.g., the total absence of pathology of a condition in a subject. The prevention may also be partial, such that the occurrence of pathology of a condition in a subject is less than that which would have occurred without the present disclosure.

“Administering” and its grammatical equivalents as used herein can refer to providing pharmaceutical compositions described herein to a subject or a patient. Conventional methods, known to those of ordinary skill in the art of medicine, can be used to administer the composition to the subject, depending upon the type of disease to be treated or the site of the disease. For example, the composition can be administered, e.g., orally, parenterally, by inhalation spray, topically, rectally, nasally, buccally, vaginally, via an implanted reservoir, or via infusion. One or more such routes can be employed.

The terms “pharmaceutical composition” and its grammatical equivalents as used herein can refer to a mixture or solution comprising a therapeutically effective amount of an active pharmaceutical ingredient together with one or more pharmaceutically acceptable excipients, carriers, and/or a therapeutic agent to be administered to a subject, e.g., a human in need thereof.

The term “pharmaceutically acceptable” and its grammatical equivalents as used herein can refer to an attribute of a material which is useful in preparing a pharmaceutical composition that is generally safe, non-toxic, and neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic, i.e., the material may be administered to a subject without causing undesirable biological effects or interacting in a deleterious manner with any of the components of the pharmaceutical composition in which it is contained.

A “pharmaceutically acceptable excipient” refers to an excipient that can be administered to a subject, together with an agent, and which does not destroy the pharmacological activity thereof and is nontoxic when administered in doses sufficient to deliver a therapeutic amount of the agent.

The term “therapeutic agent” can refer to any agent that, when administered to a subject, has a therapeutic, diagnostic, and/or prophylactic effect and/or elicits a desired biological and/or pharmacological effect. Therapeutic agents can also be referred to as “actives” or “active agents.” Such agents include, but are not limited to, cytotoxins, radioactive ions, chemotherapeutic agents, small molecule drugs, proteins, and nucleic acids.

It is appreciated that certain features of the polynucleic acid molecules, and/or polynucleic acid molecule conjugates, pharmaceutical composition comprising the polynucleic acid molecules or the polynucleic acid molecule conjugates, methods and other aspects, which are, for clarity, described in the context of separate embodiments, may also be provided in combination in a single embodiment. Conversely, various features of the polynucleic acid molecules, and/or polynucleic acid molecule conjugates, pharmaceutical composition comprising the polynucleic acid molecules or the polynucleic acid molecule conjugates, methods and other aspects, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present polynucleic acid molecules, and/or polynucleic acid molecule conjugates, pharmaceutical composition comprising the polynucleic acid molecules or the polynucleic acid molecule conjugates, methods and other aspects and are disclosed herein just as if each and every such sub-combination was individually and explicitly disclosed herein.

As used herein, the term “sense strand” can be interchangeably used with the term “passenger strand”, and the tern “antisense strand” can be interchangeably used with the term “guide strand”.

As used herein, the term “consecutive sequence” refers to a sequence contains a number of consecutive nucleotides from a reference sequence. For example, if a reference sequence is N₁N₂N₃N₄N₅N₆N₇, a consecutive sequence can be N₁N₂N₃N₄ or N₃N₄N₅N₆, but a sequence of N₁N₃N₄N₅ or N₃N₄N₇ cannot be a consecutive sequence.

As used herein, the term “negative control” refers to a subject or a cell receiving no treatment or placebo.

### Polynucleic Acid Molecules

### Target Regions of Polynucleic Acid Molecules

Described herein is a polynucleic acid molecule for modulating expression of FXI gene. In some instances, the polynucleic acid molecule comprises a single-stranded nucleic acid molecule that hybridizes to certain regions of mRNA. In some instances, the polynucleic acid molecule is a double-stranded nucleic acid molecule. Also described herein is a polynucleic acid molecule for modulating expression of FXI gene, wherein the polynucleic acid molecule is a double-stranded nucleic acid molecule, which comprises a sense strand and an antisense strand, and the antisense strand hybridizes to certain regions of FXI mRNA.

In some aspects, the polynucleic acid molecule described herein hybridizes to certain regions of human FXI mRNA. In some instances, the human FXI mRNA is NM_000128.4. In some aspects, the polynucleic acid molecule described herein hybridizes to certain regions of non-human FXI mRNA.

In some aspects, the polynucleic acid molecule described herein hybridizes to the 5′ UTR region of human FXI mRNA. In some aspects, the polynucleic acid molecule described herein hybridizes to the coding region of human FXI mRNA. In some aspects, the polynucleic acid molecule described herein hybridizes to a portion of exon 1 of human FXI mRNA. In some aspects, the polynucleic acid molecule described herein hybridizes to a portion of exon 2 of human FXI mRNA. In some aspects, the polynucleic acid molecule described herein hybridizes to a portion of exon 3 of human FXI mRNA. In some aspects, the polynucleic acid molecule described herein hybridizes to a portion of exon 4 of human FXI mRNA. In some aspects, the polynucleic acid molecule described herein hybridizes to a portion of exon 5 of human FXI mRNA. In some aspects, the polynucleic acid molecule described herein hybridizes to a portion of exon 6 of human FXI mRNA. In some aspects, the polynucleic acid molecule described herein hybridizes to a portion of exon 7 of human FXI mRNA. In some aspects, the polynucleic acid molecule described herein hybridizes to a portion of exon 8 of human FXI mRNA. In some aspects, the polynucleic acid molecule described herein hybridizes to a portion of exon 9 of human FXI mRNA. In some aspects, the polynucleic acid molecule described herein hybridizes to a portion of exon 10 of human FXI mRNA. In some aspects, the polynucleic acid molecule described herein hybridizes to a portion of exon 11 of human FXI mRNA. In some aspects, the polynucleic acid molecule described herein hybridizes to a portion of exon 12 of human FXI mRNA. In some aspects, the polynucleic acid molecule described herein hybridizes to a portion of exon 13 of human FXI mRNA. In some aspects, the polynucleic acid molecule described herein hybridizes to a portion of exon 14 of human FXI mRNA. In some aspects, the polynucleic acid molecule described herein hybridizes to a portion of exon 15 of human FXI mRNA. In some aspects, the polynucleic acid molecule described herein hybridizes to the 3′ UTR region of human FXI mRNA.

In some aspects, the target region that the polynucleic acid molecule described herein hybridizes to is determined by FXI silencing effectiveness and possible off-target effects. In some instances, the start of the target region fall between positions 1-10, 11-20, 21-30, 31-40, 41-50, 51-60, 61-70, 71-80, 81-90, or 91-100 of NM_000128.4. In some instances, the start of the target region fall between positions 101-110, 111-120, 121-130, 131-140, 141-150, 151-160, 161-170, 171-180, 181-190, or 191-200 of NM_000128.4. In some instances, the start of the target region fall between positions of 201-210, 211-220, 221-230, 231-240, 241-250, 251-260, 261-270, 271-280, 281-290, or 291-300 of NM_000128.4. In some instances, the start of the target region fall between positions 301-310, 311-320, 321-330, 331-340, 341-350, 351-360, 361-370, 371-380, 381-390, or 391-400 of NM_000128.4. In some instances, the start of the target region fall between positions 401-410, 411-420, 421-430, 431-440, 441-450, 451-460, 461-470, 471-480, 481-490, or 491-500 of NM_000128.4. In some instances, the start of the target region fall between positions 501-510, 511-520, 521-530, 531-540, 541-550, 551-560, 561-570, 571-580, 581-590, or 591-600 of NM_000128.4. In some instances, the start of the target region fall between positions 601-610, 611-620, 621-630, 631-640, 641-650, 651-660, 661-670, 671-680, 681-690, or 691-700 of NM_000128.4. In some instances, the start of the target region fall between positions 701-710, 711-720, 721-730, 731-740, 741-750, 751-760, 761-770, 771-780, 781-790, or 791-800 of NM_000128.4. In some instances, the start of the target region fall between positions 801-810, 811-820, 821-830, 831-840, 841-850, 851-860, 861-870, 871-880, 881-890, or 891-900 of NM_000128.4. In some instances, the start of the target region fall between positions 901-910, 911-920, 921-930, 931-940, 941-950, 951-960, 961-970, 971-980, 981-990, or991-1000 of NM_000128.4. In some instances, the start of the target region fall between positions 1001-1010, 1011-1020, 1021-1030, 1031-1040, 1041-1050, 1051-1060, 1061-1070, 1071-1080, 1081-1090, or 1091-1100 of NM_000128.4. In some instances, the start of the target region fall between positions 1101-1110, 1111-1120, 1121-1130, 1131-1140, 1141-1150, 1151-1160, 1161-1170, 1171-1180, 1181-1190, or 1191-1200 of NM_000128.4. In some instances, the start of the target region fall between positions 1201-1210, 1211-1220, 1221-1230, 1231-1240, 1241-1250, 1251-1260, 1261-1270, 1271-1280, 1281-1290, or 1291-1300 of NM_000128.4. In some instances, the start of the target region fall between positions 1301-1310, 1311-1320, 1321-1330, 1331-1340, 1341-1350, 1351-1360, 1361-1370, 1371-1380, 1381-1390, or 1391-1400 of NM_000128.4. In some instances, the start of the target region fall between positions 1401-1410, 1411-1420, 1421-1430, 1431-1440, 1441-1450, 1451-1460, 1461-1470, 1471-1480, 1481-1490, or 1491-1500 of NM_000128.4. In some instances, the start of the target region fall between positions 1501-1510, 1511-1520, 1521-1530, 1531-1540, 1541-1550, 1551-1560, 1561-1570, 1571-1580, 1581-1590, or 1591-1600 of NM_000128.4. In some instances, the start of the target region fall between positions 1601-1610, 1611-1620, 1621-1630, 1631-1640, 1641-1650, 1651-1660, 1661-1670, 1671-1680, 1681-1690, or 1691-1700 of NM_000128.4. In some instances, the start of the target region fall between positions 1701-1710, 1711-1720, 1721-1730, 1731-1740, 1741-1750, 1751-1760, 1761-1770, 1771-1780, 1781-1790, or 1791-1800 of NM_000128.4. In some instances, the start of the target region fall between positions 1801-1810, 1811-1820, 1821-1830, 1831-1840, 1841-1850, 1851-1860, 1861-1870, 1871-1880, 1881-1890, or 1891-1900 of NM_000128.4. In some instances, the start of the target region fall between positions 1901-1910, 1911-1920, 1921-1930, 1931-1940, 1941-1950, 1951-1960, 1961-1970, 1971-1980, 1981-1990, or 1991-2000 of NM_000128.4. In some instances, the start of the target region fall between positions 2001-2010, 2011-2020, 2021-2030, 2031-2040, 2041-2050, 2051-2060, 2061-2070, 2071-2080, 2081-2090, or 2091-2100 of NM_000128.4. In some instances, the start of the target region fall between positions 2101-2110, 2111-2120, 2121-2130, 2131-2140, 2141-2150, 2151-2160, 2161-2170, 2171-2180, 2181-2190, or 2191-2200 of NM_000128.4. In some instances, the start of the target region fall between positions 2201-2210, 2211-2220, 2221-2230, 2231-2240, 2241-2250, 2251-2260, 2261-2270, 2271-2280, 2281-2290, or 2291-2300 of NM_000128.4. In some instances, the start of the target region fall between positions 2301-2310, 2311-2320, 2321-2330, 2331-2340, 2341-2350, 2351-2360, 2361-2370, 2371-2380, 2381-2390, or 2391-2400 of NM_000128.4. In some instances, the start of the target region fall between positions 2401-2410, 2411-2420, 2421-2430, 2431-2440, 2441-2450, 2451-2460, 2461-2470, 2471-2480, 2481-2490, or 2491-2500 of NM_000128.4. In some instances, the start of the target region fall between positions 2501-2510, 2511-2520, 2521-2530, 2531-2540, 2541-2550, 2551-2560, 2561-2570, 2571-2580, 2581-2590, or 2591-2600 of NM_000128.4. In some instances, the start of the target region fall between positions 2601-2610, 2611-2620, 2621-2630, 2631-2640, 2641-2650, 2651-2660, 2661-2670, 2671-2680, 2681-2690, or 2691-2700 of NM_000128.4. In some instances, the start of the target region fall between positions 2701-2710, 2711-2720, 2721-2730, 2731-2740, 2741-2750, 2751-2760, 2761-2770, 2771-2780, 2781-2790, or 2791-2800 of NM_000128.4. In some instances, the start of the target region fall between positions 2801-2810, 2811-2820, 2821-2830, 2831-2840, 2841-2850, 2851-2860, 2861-2870, 2871-2880, 2881-2890, or 2891-2900 of NM_000128.4. In some instances, the start of the target region fall between positions 2901-2910, 2911-2920, 2921-2930, 2931-2940, 2941-2950, 2951-2960, 2961-2970, 2971-2980, 2981-2990, or 2991-3000 of NM_000128.4. In some instances, the start of the target region fall between positions 3001-3010, 3011-3020, 3021-3030, 3031-3040, 3041-3050, or 3051-3053 of NM_000128.4.

### Structure of Polynucleic Acid Molecules

### Single-Stranded Nucleic Acid Molecule

Described herein is a polynucleic acid molecule for modulating expression of FXI gene, wherein the polynucleic acid molecule comprises a single-stranded nucleic acid molecule that is reverse complementary to the target region of FXI mRNA as described above.

In some aspects, the polynucleic acid molecule described herein is not 100% complementary to the target region of FXI mRNA. Accordingly, in some instances, the polynucleic acid molecule described herein is about 95% complementary to the target region of FXI mRNA. In some instances, the polynucleic acid molecule described herein is about 90% complementary to the target region of FXI mRNA. In some instances, the polynucleic acid molecule described herein is about 85% complementary to the target region of FXI mRNA. In some instances, the polynucleic acid molecule described herein is about 80% complementary to the target region of FXI mRNA. In some instances, the polynucleic acid molecule described herein is about 75% complementary to the target region of FXI mRNA. In some instances, the polynucleic acid molecule described herein is about 70% complementary to the target region of FXI mRNA.

In some aspects, the polynucleic acid molecule described herein comprises a nucleic acid sequence in Tables 1-2 and Tables 7-9. In some instances, the polynucleic acid molecule described herein comprises a nucleic acid sequence that is at least 80%, at least 85%, at least 90%, or at least 95% complementary to a sequence in Tables 1-2 and Tables 7-9. In some instances, the polynucleic acid molecule described herein comprises a nucleic acid sequence that is at least 80%, at least 85%, at least 90%, or at least 95% complementary to a nucleic acid sequence selected from SEQ ID NOs: 101-150 and 220. In some instances, the polynucleic acid molecule described herein comprises a nucleic acid sequence that is at least 80%, at least 85%, at least 90%, or at least 95% complementary to a nucleic acid sequence selected from SEQ ID NOs: 104, 106-107, 109-111, 113-114, 135, 150, and 220.

In some instances, the polynucleic acid molecule described herein comprises a nucleic acid sequence that is at least 80%, at least 85%, at least 90%, or at least 95% complementary to a sequence in Tables 1-2 and Tables 7-9, excluding overhangs. In some instances, the polynucleic acid molecule described herein comprises a nucleic acid sequence that is at least 80%, at least 85%, at least 90%, or at least 95% complementary to a nucleic acid sequence selected from SEQ ID NOs: 101-150 and 220, excluding overhangs. In some instances, the polynucleic acid molecule described herein comprises a nucleic acid sequence that is at least 80%, at least 85%, at least 90%, or at least 95% complementary to a nucleic acid sequence selected from SEQ ID NOs: 104, 106-107, 109-111, 113-114, 135, 150, and 220, excluding overhangs.

In yet other aspects, the polynucleic acid molecule described herein comprises a nucleic acid sequence comprising 14 consecutive nucleotides that are complementary to a sequence in Tables 1-2 and Tables 7-9 with no more than 1, 2, 3, or 4 mismatches. In some aspects, the polynucleic acid molecule described herein comprises a nucleic acid sequence comprising 14 consecutive nucleotides that are complementary to a nucleic acid sequence of SEQ ID NOs: 101-150 and 220 with no more than 1, 2, 3, or 4 mismatches. In yet other aspects, the polynucleic acid molecule described herein comprises a nucleic acid sequence comprising 15 consecutive nucleotides that are complementary to a sequence in Tables 1-2 and Tables 7-9 with no more than 1, 2, 3, or 4 mismatches. In some aspects, the polynucleic acid molecule described herein comprises a nucleic acid sequence comprising 15 consecutive nucleotides that are complementary to a nucleic acid sequence of SEQ ID NOs: 101-150 and 220 with no more than 1, 2, 3, or 4 mismatches. In yet other aspects, the polynucleic acid molecule described herein comprises a nucleic acid sequence comprising 16 consecutive nucleotides that are complementary to a sequence in Tables 1-2 and Tables 7-9 with no more than 1, 2, 3, or 4 mismatches. In some aspects, the polynucleic acid molecule described herein comprises a nucleic acid sequence comprising 16 consecutive nucleotides that are complementary to a nucleic acid sequence of SEQ ID NOs: 101-150 and 220 with no more than 1, 2, 3, or 4 mismatches. In yet other aspects, the polynucleic acid molecule described herein comprises a nucleic acid sequence comprising 17 consecutive nucleotides that are complementary to a sequence in Tables 1-2 and Tables 7-9 with no more than 1, 2, 3, or 4 mismatches. In some aspects, the polynucleic acid molecule described herein comprises a nucleic acid sequence comprising 17 consecutive nucleotides that are complementary to a nucleic acid sequence of SEQ ID NOs: 101-150 and 220 with no more than 1, 2, 3, or 4 mismatches. In yet other aspects, the polynucleic acid molecule described herein comprises a nucleic acid sequence comprising 18 consecutive nucleotides that are complementary to a sequence in Tables 1-2 and Tables 7-9 with no more than 1, 2, 3, or 4 mismatches. In some aspects, the polynucleic acid molecule described herein comprises a nucleic acid sequence comprising 18 consecutive nucleotides that are complementary to a nucleic acid sequence of SEQ ID NOs: 101-150 and 220 with no more than 1, 2, 3, or 4 mismatches. In yet other aspects, the polynucleic acid molecule described herein comprises a nucleic acid sequence comprising 19 consecutive nucleotides that are complementary to a sequence in Tables 1-2 and Tables 7-9 with no more than 1, 2, 3, or 4 mismatches. In some aspects, the polynucleic acid molecule described herein comprises a nucleic acid sequence comprising 19 consecutive nucleotides that are complementary to a nucleic acid sequence of SEQ ID NOs: 101-150 and 220 with no more than 1, 2, 3, or 4 mismatches. In yet other aspects, the polynucleic acid molecule described herein comprises a nucleic acid sequence comprising 20 consecutive nucleotides that are complementary to a sequence in Tables 1-2 and Tables 7-9 with no more than 1, 2, 3, or 4 mismatches. In some aspects, the polynucleic acid molecule described herein comprises a nucleic acid sequence comprising 20 consecutive nucleotides that are complementary to a nucleic acid sequence of SEQ ID NOs: 101-150 and 220 with no more than 1, 2, 3, or 4 mismatches. In yet other aspects, the polynucleic acid molecule described herein comprises a nucleic acid sequence comprising 21 consecutive nucleotides that are complementary to a sequence in Tables 1-2 and Tables 7-9 with no more than 1, 2, 3, or 4 mismatches. In some aspects, the polynucleic acid molecule described herein comprises a nucleic acid sequence comprising 21 consecutive nucleotides that are complementary to a nucleic acid sequence of SEQ ID NOs: 101-150 and 220 with no more than 1, 2, 3, or 4 mismatches. In yet other aspects, the polynucleic acid molecule described herein comprises a nucleic acid sequence comprising 22 consecutive nucleotides that are complementary to a sequence in Tables 1-2 and Tables 7-9 with no more than 1, 2, 3, or 4 mismatches. In some aspects, the polynucleic acid molecule described herein comprises a nucleic acid sequence comprising 22 consecutive nucleotides that are complementary to a nucleic acid sequence of SEQ ID NOs: 101-150 and 220 with no more than 1, 2, 3, or 4 mismatches.

In some aspects, the polynucleic acid molecule described herein comprises a nucleic acid sequence comprising 14 consecutive nucleotides that are complementary to a nucleic acid sequence of SEQ ID NOs: 104, 106-107, 109-111, 113-114, 135, 150, and 220 with no more than 1, 2, 3, or 4 mismatches. In some aspects, the polynucleic acid molecule described herein comprises a nucleic acid sequence comprising 15 consecutive nucleotides that are complementary to a nucleic acid sequence of SEQ ID NOs: 104, 106-107, 109-111, 113-114, 135, 150, and 220 with no more than 1, 2, 3, or 4 mismatches. In some aspects, the polynucleic acid molecule described herein comprises a nucleic acid sequence comprising 16 consecutive nucleotides that are complementary to a nucleic acid sequence of SEQ ID NOs: 104, 106-107, 109-111, 113-114, 135, 150, and 220 with no more than 1, 2, 3, or 4 mismatches. In some aspects, the polynucleic acid molecule described herein comprises a nucleic acid sequence comprising 17 consecutive nucleotides that are complementary to a nucleic acid sequence of SEQ ID NOs: 104, 106-107, 109-111, 113-114, 135, 150, and 220 with no more than 1, 2, 3, or 4 mismatches. In some aspects, the polynucleic acid molecule described herein comprises a nucleic acid sequence comprising 18 consecutive nucleotides that are complementary to a nucleic acid sequence of SEQ ID NOs: 104, 106-107, 109-111, 113-114, 135, 150, and 220 with no more than 1, 2, 3, or 4 mismatches. In some aspects, the polynucleic acid molecule described herein comprises a nucleic acid sequence comprising 19 consecutive nucleotides that are complementary to a nucleic acid sequence of SEQ ID NOs: 104, 106-107, 109-111, 113-114, 135, 150, and 220 with no more than 1, 2, 3, or 4 mismatches. In some aspects, the polynucleic acid molecule described herein comprises a nucleic acid sequence comprising 20 consecutive nucleotides that are complementary to a nucleic acid sequence of SEQ ID NOs: 104, 106-107, 109-111, 113-114, 135, 150, and 220 with no more than 1, 2, 3, or 4 mismatches. In some aspects, the polynucleic acid molecule described herein comprises a nucleic acid sequence comprising 21 consecutive nucleotides that are complementary to a nucleic acid sequence of SEQ ID NOs: 104, 106-107, 109-111, 113-114, 135, 150, and 220 with no more than 1, 2, 3, or 4 mismatches. In some aspects, the polynucleic acid molecule described herein comprises a nucleic acid sequence comprising 22 consecutive nucleotides that are complementary to a nucleic acid sequence of SEQ ID NOs: 104, 106-107, 109-111, 113-114, 135, 150, and 220 with no more than 1, 2, 3, or 4 mismatches.

In yet other aspects, the polynucleic acid molecule described herein comprises a nucleic acid sequence comprising 14, 15, 16, 17, 18, 19, 20, 21, or 22 consecutive nucleotides that are complementary to a sequence in Tables 1-2 and Tables 7-9 without overhangs with no more than 1, 2, 3, or 4 mismatches. In some aspects, the polynucleic acid molecule described herein comprises a nucleic acid sequence comprising 14, 15, 16, 17, 18, 19, 20, 21, or 22 consecutive nucleotides that are complementary to a nucleic acid sequence of SEQ ID NOs: 101-150 and 220 without overhangs with no more than 1, 2, 3, or 4 mismatches. In some aspects, the polynucleic acid molecule described herein comprises a nucleic acid sequence comprising 14, 15, 16, 17, 18, 19, 20, 21, or 22 consecutive nucleotides that are complementary to a nucleic acid sequence of SEQ ID NOs: 104, 106-107, 109-111, 113-114, 135, 150, and 220 without overhangs with no more than 1, 2, 3, or 4 mismatches.

In some aspects, the polynucleic acid molecule described herein comprises a strand of at least 10, 11, 12, 13, 14, or 15 nucleotides in length. In some aspects, the polynucleic acid molecule described herein comprises a strand of about 15-40, 16-30, 17-30, 18-30, 18-27, 18-25, 18-23, 19-23, 20-23, or 21-23 nucleotides in length. In some aspects, the polynucleic acid molecule described herein comprises a strand of about 15, 16, 17, 18, 19, 20 nucleotides long. In some aspects, the polynucleic acid molecule described herein comprises a strand of about 21, 22, 23, 24, 25 nucleotides long. In some aspects, the polynucleic acid molecule described herein comprises a strand of about 26, 27, 28, 29, 30 nucleotides long.

In some aspects, the polynucleic acid molecule described herein comprises a single-stranded nucleic acid of at least 10, 11, 12, 13, 14, or 15 nucleotides in length. In some aspects, the polynucleic acid molecule described herein comprises a single-stranded nucleic acid of about 15-30, 16-30, 17-30, 18-30, 18-27, 18-25, 18-23, 19-23, 20-23, or 21-23 nucleotides in length.

In some aspects, the polynucleic acid molecule described herein comprises a single-stranded nucleic acid of about 15, 16, 17, 18, 19, 20 nucleotides long. In some aspects, the polynucleic acid molecule described herein comprises a single-stranded nucleic acid of about 21, 22, 23, 24, 25 nucleotides long. In some aspects, the polynucleic acid molecule described herein comprises a single-stranded nucleic acid of about 26, 27, 28, 29, 30 nucleotides long. In some aspects, the polynucleic acid molecule described herein comprises a single-stranded nucleic acid of about 21 nucleotides long. In some aspects, the polynucleic acid molecule described herein comprises a single-stranded nucleic acid of about 23 nucleotides long.

### Double-Stranded Nucleic Acid Molecule

Further described herein is a polynucleic acid molecule for modulating expression of FXI gene, wherein the polynucleic acid molecule is a double-stranded molecule that comprises a sense strand and an antisense strand, and the antisense strand is reverse complementary to the target region of FXI mRNA as described above.

In some aspects, the antisense strand described herein is 100% complementary to the target region of FXI mRNA. In other aspects, the antisense strand described herein is not 100% complementary to the target region of FXI mRNA. Accordingly, in some instances, the antisense strand described herein is about 95% complementary to the target region of FXI mRNA. In some aspects, the antisense strand described herein is about 90% complementary to the target region of FXI mRNA. In some aspects, the antisense strand described herein is about 85% complementary to the target region of FXI mRNA. In some aspects, the antisense strand described herein is about 80% complementary to the target region of FXI mRNA. In some aspects, the antisense strand described herein is about 75% complementary to the target region of FXI mRNA. In some aspects, the antisense strand described herein is about 70% complementary to the target region of FXI mRNA.

In some aspects, the polynucleic acid molecule described herein comprises a nucleic acid sequence in Tables 1-2 and Tables 7-9. In other aspects, the polynucleic acid molecule described herein comprises a nucleic acid sequence that is at least 80%, at least 85%, at least 90%, or at least 95% identical to a sequence in Tables 1-2 and Tables 7-9. In some instances, the sense strand described herein comprises a nucleic acid sequence that is at least 80%, at least 85%, at least 90%, or at least 95% identical to a nucleic acid sequence selected from SEQ ID NOs: 101-150 and 220. In some instances, the antisense strand described herein comprises a nucleic acid sequence that is at least 80%, at least 85%, at least 90%, or at least 95% identical to a nucleic acid sequence selected from SEQ ID NOs: 1-50 and 215-216. In some instances, the sense strand described herein comprises a nucleic acid sequence that is at least 80%, at least 85%, at least 90%, or at least 95% identical to a nucleic acid sequence selected from SEQ ID NOs: 104, 106-107, 109-111, 113-114, 135, 150, and 220. In some instances, the antisense strand described herein comprises a nucleic acid sequence that is at least 80%, at least 85%, at least 90%, or at least 95% identical to a nucleic acid sequence selected from SEQ ID NOs: 4, 6-7,9-11, 13-14, 35, 50, and 215-216.

In yet other aspects, the polynucleic acid molecule described herein comprises a nucleic acid sequence comprising 14 consecutive sequences out of the sequences in Tables 1-2 and Tables 7-9 with no more than 1, 2, 3, or 4 mismatches. In some aspects, the sense strand described herein comprises a nucleic acid sequence comprising 14 consecutive sequences of SEQ ID NOs: 101-150 and 220 with no more than 1, 2, 3, or 4 mismatches. In some aspects, the antisense strand described herein comprises a nucleic acid sequence comprising 14 consecutive sequences of SEQ ID NOs: 1-50 and 215-216 with no more than 1, 2, 3, or 4 mismatches. In yet other aspects, the polynucleic acid molecule described herein comprises a nucleic acid sequence comprising 15 consecutive sequences out of the sequences in Tables 1-2 and Tables 7-9 with no more than 1, 2, 3, or 4 mismatches. In some aspects, the sense strand described herein comprises a nucleic acid sequence comprising 15 consecutive sequences of SEQ ID NOs: 101-150 and 220 with no more than 1, 2, 3, or 4 mismatches. In some aspects, the antisense strand described herein comprises a nucleic acid sequence comprising 15 consecutive sequences of SEQ ID NOs: 1-50 and 215-216 with no more than 1, 2, 3, or 4 mismatches. In yet still other aspects, the polynucleic acid molecule described herein comprises a nucleic acid sequence comprising 16 consecutive sequences out of the sequences in Tables 1-2 and Tables 7-9 with no more than 1, 2, 3, or 4 mismatches. In some aspects, the sense strand described herein comprises a nucleic acid sequence comprising 16 consecutive sequences of SEQ ID NOs: 101-150 and 220 with no more than 1, 2, 3, or 4 mismatches. In some aspects, the antisense strand described herein comprises a nucleic acid sequence comprising 16 consecutive sequences of SEQ ID NOs: 1-50 and 215-216 with no more than 1, 2, 3, or 4 mismatches. In yet still other aspects, the polynucleic acid molecule described herein comprises a nucleic acid sequence comprising 17 consecutive sequences out of the sequences in Tables 1-2 and Tables 7-9 with no more than 1, 2, 3, or 4 mismatches. In some aspects, the sense strand described herein comprises a nucleic acid sequence comprising 17 consecutive sequences of SEQ ID NOs: 101-150 and 220 with no more than 1, 2, 3, or 4 mismatches. In some aspects, the antisense strand described herein comprises a nucleic acid sequence comprising 17 consecutive sequences of SEQ ID NOs: 1-50 and 215-216 with no more than 1, 2, 3, or 4 mismatches. In yet other aspects, the polynucleic acid molecule described herein comprises a nucleic acid sequence comprising 18 consecutive sequences out of the sequences in Tables 1-2 and Tables 7-9 with no more than 1, 2, 3, or 4 mismatches. In some aspects, the sense strand described herein comprises a nucleic acid sequence comprising 18 consecutive sequences of SEQ ID NOs: 101-150 and 220 with no more than 1, 2, 3, or 4 mismatches. In some aspects, the antisense strand described herein comprises a nucleic acid sequence comprising 18 consecutive sequences of SEQ ID NOs: 1-50 and 215-216 with no more than 1, 2, 3, or 4 mismatches. In yet other aspects, the polynucleic acid molecule described herein comprises a nucleic acid sequence comprising 19 consecutive sequences out of the sequences in Tables 1-2 and Tables 7-9 with no more than 1, 2, 3, or 4 mismatches. In some aspects, the sense strand described herein comprises a nucleic acid sequence comprising 19 consecutive sequences of SEQ ID NOs: 101-150 and 220 with no more than 1, 2, 3, or 4 mismatches. In some aspects, the antisense strand described herein comprises a nucleic acid sequence comprising 19 consecutive sequences of SEQ ID NOs: 1-50 and 215-216 with no more than 1, 2, 3, or 4 mismatches. In yet other aspects, the polynucleic acid molecule described herein comprises a nucleic acid sequence comprising 20 consecutive sequences out of the sequences in Tables 1-2 and Tables 7-9 with no more than 1, 2, 3, or 4 mismatches. In some aspects, the sense strand described herein comprises a nucleic acid sequence comprising 20 consecutive sequences of SEQ ID NOs: 101-150 and 220 with no more than 1, 2, 3, or 4 mismatches. In some aspects, the antisense strand described herein comprises a nucleic acid sequence comprising 20 consecutive sequences of SEQ ID NOs: 1-50 and 215-216 with no more than 1, 2, 3, or 4 mismatches. In yet other aspects, the polynucleic acid molecule described herein comprises a nucleic acid sequence comprising 21 consecutive sequences out of the sequences in Tables 1-2 and Tables 7-9 with no more than 1, 2, 3, or 4 mismatches. In some aspects, the sense strand described herein comprises a nucleic acid sequence comprising 21 consecutive sequences of SEQ ID NOs: 101-150 and 220 with no more than 1, 2, 3, or 4 mismatches. In some aspects, the antisense strand described herein comprises a nucleic acid sequence comprising 21 consecutive sequences of SEQ ID NOs: 1-50 and 215-216 with no more than 1, 2, 3, or 4 mismatches. In yet other aspects, the polynucleic acid molecule described herein comprises a nucleic acid sequence comprising 22 consecutive sequences out of the sequences in Tables 1-2 and Tables 7-9 with no more than 1, 2, 3, or 4 mismatches. In some aspects, the sense strand described herein comprises a nucleic acid sequence comprising 22 consecutive sequences of SEQ ID NOs: 101-150 and 220 with no more than 1, 2, 3, or 4 mismatches. In some aspects, the antisense strand described herein comprises a nucleic acid sequence comprising 22 consecutive sequences of SEQ ID NOs: 1-50 and 215-216 with no more than 1, 2, 3, or 4 mismatches.

In some aspects, the sense strand described herein comprises a nucleic acid sequence comprising 15 consecutive sequences of SEQ ID NOs: 104, 106-107, 109-111, 113-114, 135, 150, and 220 with no more than 1, 2, 3, or 4 mismatches. In some aspects, the antisense strand described herein comprises a nucleic acid sequence comprising 15 consecutive sequences of SEQ ID NOs: 4, 6-7, 9-11, 13-14, 35, 50, and 215-216 with no more than 1, 2, 3, or 4 mismatches. In some aspects, the sense strand described herein comprises a nucleic acid sequence comprising 16 consecutive sequences of SEQ ID NOs: 104, 106-107, 109-111, 113-114, 135, 150, and 220 with no more than 1, 2, 3, or 4 mismatches. In some aspects, the antisense strand described herein comprises a nucleic acid sequence comprising 16 consecutive sequences of SEQ ID NOs: 4, 6-7, 9-11, 13-14, 35, 50, and 215-216 with no more than 1, 2, 3, or 4 mismatches. In some aspects, the sense strand described herein comprises a nucleic acid sequence comprising 17 consecutive sequences of SEQ ID NOs: 104, 106-107, 109-111, 113-114, 135, 150, and 220 with no more than 1, 2, 3, or 4 mismatches. In some aspects, the antisense strand described herein comprises a nucleic acid sequence comprising 17 consecutive sequences of SEQ ID NOs: 4, 6-7, 9-11, 13-14, 35, 50, and 215-216 with no more than 1, 2, 3, or 4 mismatches. In some aspects, the sense strand described herein comprises a nucleic acid sequence comprising 18 consecutive sequences of SEQ ID NOs: 104, 106-107, 109-111, 113-114, 135, 150, and 220 with no more than 1, 2, 3, or 4 mismatches. In some aspects, the antisense strand described herein comprises a nucleic acid sequence comprising 18 consecutive sequences of SEQ ID NOs: 4, 6-7, 9-11, 13-14, 35, 50, and 215-216 with no more than 1, 2, 3, or 4 mismatches. In some aspects, the sense strand described herein comprises a nucleic acid sequence comprising 19 consecutive sequences of SEQ ID NOs: 104, 106-107, 109-111, 113-114, 135, 150, and 220 with no more than 1, 2, 3, or 4 mismatches. In some aspects, the antisense strand described herein comprises a nucleic acid sequence comprising 19 consecutive sequences of SEQ ID NOs: 4, 6-7, 9-11, 13-14, 35, 50, and 215-216 with no more than 1, 2, 3, or 4 mismatches. In some aspects, the sense strand described herein comprises a nucleic acid sequence comprising 20 consecutive sequences of SEQ ID NOs: 104, 106-107, 109-111, 113-114, 135, 150, and 220 with no more than 1, 2, 3, or 4 mismatches. In some aspects, the antisense strand described herein comprises a nucleic acid sequence comprising 20 consecutive sequences of SEQ ID NOs: 4, 6-7, 9-11, 13-14, 35, 50, and 215-216 with no more than 1, 2, 3, or 4 mismatches. In some aspects, the sense strand described herein comprises a nucleic acid sequence comprising 21 consecutive sequences of SEQ ID NOs: 104, 106-107, 109-111, 113-114, 135, 150, and 220 with no more than 1, 2, 3, or 4 mismatches. In some aspects, the antisense strand described herein comprises a nucleic acid sequence comprising 21 consecutive sequences of SEQ ID NOs: 4, 6-7, 9-11, 13-14, 35, 50, and 215-216 with no more than 1, 2, 3, or 4 mismatches. In some aspects, the sense strand described herein comprises a nucleic acid sequence comprising 22 consecutive sequences of SEQ ID NOs: 104, 106-107, 109-111, 113-114, 135, 150, and 220 with no more than 1, 2, 3, or 4 mismatches. In some aspects, the antisense strand described herein comprises a nucleic acid sequence comprising 22 consecutive sequences of SEQ ID NOs: 4, 6-7, 9-11, 13-14, 35, 50, and 215-216 with no more than 1, 2, 3, or 4 mismatches.

In some aspects, the polynucleic acid molecule described herein comprises a strand and an antisense of at least 10, 11, 12, 13, 14, or 15 nucleotides in length. In some aspects, the polynucleic acid molecule described herein comprises a sense and an antisense strand of about 15-30, 16-30, 17-30, 18-30, 18-27, 18-25, 18-23, 19-23, 20-23, or 21-23 nucleotides in length. In some aspects, the polynucleic acid molecule described herein comprises a sense and an antisense strand of about 15, 16, 17, 18, 19, 20 nucleotides long. In some aspects, the polynucleic acid molecule described herein comprises a sense and an antisense strand of about 21, 22, 23, 24, 25 nucleotides long. In some aspects, the polynucleic acid molecule described herein comprises a sense and an antisense strand of about 26, 27, 28, 29, 30 nucleotides long. In some aspects, the polynucleic acid molecule described herein comprises a sense strand of 19 nucleotides long, and an antisense strand of about 21 nucleotides long. In some aspects, the polynucleic acid molecule described herein comprises a sense strand of 21 nucleotides long, and an antisense strand of about 23 nucleotides long.

In some aspects, the sense strand and the antisense strand described herein are reverse complementary to each other and form a duplex with a 3′ overhang on the antisense strand. In some aspects, the sense strand and the antisense strand described herein are reverse complementary to each other and form a duplex with a 5′ overhang on the antisense strand. In some aspects, the sense strand and the antisense strand described herein are reverse complementary to each other and form a duplex with a 3′ overhang on the sense strand. In some aspects, the sense strand and the antisense strand described herein are reverse complementary to each other and form a duplex with a 5′ overhang on the sense strand.

### Modifications of Polynucleic Acid Molecules

In some aspects, described herein is the polynucleic acid molecule described herein with modifications. In some aspects, the modifications described herein occurs one or more different structures of the polynucleotide molecule described herein (e.g., modifications on sugar ring(s), backbone(s), base(s)). In some aspects, the modifications described herein comprise substitutions of one or more nucleotide in the polynucleic acid molecule described herein. In some aspects, different percentages of the polynucleic acid molecule described herein comprise the modifications described herein. In some aspects, different positions of the polynucleic acid molecule described herein comprise the modifications described herein. In some aspects, the modifications described herein comprise modification patterns disclosed in WO/2018/035380, which is herein incorporated by reference in its entirety.

### Types of Modifications

In some aspects, the polynucleotide molecule described herein comprises one or more sugar-modified nucleotide. In some aspects, the sugar-modified nucleotide is a 2′-fluoro modified nucleotide. In some instances, the sugar-modified nucleotide includes a modification at a 2′ hydroxyl group of the ribose moiety. In some instances, the sugar-modified nucleotide includes modification with an H, OR, R, halo, SH, SR, NH2, NHR, NR2, or CN, wherein R is an alkyl moiety. In some aspects, the sugar-modified nucleotide is a 2′-O-methyl modified nucleotide or 2′-alkoxy modified nucleotide (e.g., 2′-methoxy modified nucleotide). In some instances, 2′ hydroxyl group modification includes 2′-deoxy, 2′-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In some instances, 2′ hydroxyl group of the ribose moiety includes a locked or bridged ribose modification (e.g., LNA), an unlocked ribose modification (e.g., UNA), or ethylene nucleic acids (ENA). In some instances, the alkyl moiety comprises a hetero substitution. In some instances, the carbon of the heterocyclic group is substituted by a nitrogen, oxygen or sulfur. In some aspects, the sugar-modified nucleotide is a 2′-amino modified nucleotide. In some aspects, the sugar-modified nucleotide is a 2′-azido modified nucleotide. In some aspects, the sugar-modified nucleotide is a 2′-deoxy modified nucleotide. In some aspects, the sugar-modified nucleotide is a 2′-O-methoxythyl (2′-MOE). In some aspects, the sugar-modified nucleotide is a locked nucleic acid (LNA). In some aspects, the sugar-modified nucleotide is an ethylene-bridged nucleic acid (ENA). In some aspects, the sugar-modified nucleotide is a (S)-constrained ethyl (cEt). In some aspects, the sugar-modified nucleotide is a tricyclo-DNA (tcDNA). In some aspects, the sugar-modified nucleotide is a 2′-NH₂ nucleic acid.

In some aspects, the polynucleotide molecule described herein comprises one or more sugarphosphate-modified nucleotide. In some aspects, the modified sugarphosphate is phosphorodiamidate morpholino (PMO). In some aspects, the modified sugarphosphate is phosphoramidate. In some instances, the heterocyclic substitution includes imidazole, and pyrrolidino. In some aspects, the modified sugarphosphate is thiophosphoramidate. In some aspects, the modified sugarphosphate is peptide nucleic acid (PNA).

In some aspects, the poly nucleotide molecule described herein comprises one or more backbone-modified nucleotide. In some aspects, the modified backbone is a methylphosphonate. In some aspects, the modified backbone is phosphorothioate. In some aspects, the modified backbone is a guanidinopropyl phosphoramidate. In some aspects, the modified backbone is a mesyl-phosphoramidate (MsPA) linkages. In some instances, the modified backbone comprises one or more of phosphorodithioates, methylphosphonates, 5′-alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates, phosphoramidates.

In some instances, one or more phosphorothioate internucleotide linkages are located at the 5′ end of the guide strand. In some cases, two phosphorothioate internucleotide linkages are located between the nucleotides at positions 1, 2, and 3 from the 5′-end of the guide strand. In some cases, one or more phosphorothioate internucleotide linkages are located at the 3′ end of the guide strand. In some cases, two phosphorothioate internucleotide linkages are located between the nucleotides at positions 1, 2, and 3 from the 3′-end of the guide strand. In some cases, two phosphorothioate internucleotide linkages are located between the nucleotides at positions 1, 2, and 3 from the 5′-end of the guide strand and two phosphorothioate internucleotide linkages are located between the nucleotides at positions 1, 2, and 3 from the 3′ end of the guide strand. In some cases, the guide strand comprises phosphorothioate internucleotide linkages between the nucleotides at positions 1, 2, 3, 4 (between 1 and 2, between 2 and 3, and between 3 and 4) of the guide strand and between the nucleotides at positions 1, 2, and 3 (between 1 and 2, and between 2 and 3) from the 3′-end of the guide strand.

In some instances, more phosphorothioate internucleotide linkages are located at the 5′ end of the passenger strand. In some cases, two phosphorothioate internucleotide linkages are located between the nucleotides at positions 1, 2, and 3 from the 5′-end of the passenger strand. In some cases, two phosphorothioate internucleotide linkages are located between the nucleotides at positions 1, 2, and 3 from the 5′-end of the passenger strand, and two phosphorothioate internucleotide linkages are located between the nucleotides at positions 1, 2, and 3 from the 5′-end of the guide strand and two phosphorothioate internucleotide linkages are located between the nucleotides at positions 1, 2, and 3 from the 3′-end of the guide strand.

In some instances, one or more phosphorothioate internucleotide linkages are located in the seed region of the guide strand. In some cases, the guide strand comprises a phosphorothioate internucleotide linkage between the nucleotides at positions 5 and 6 from the 5′-end of the guide strand. In some cases, the guide strand comprises a phosphorothioate internucleotide linkage between the nucleotides 6 and 7 from the 3′-end of the guide strand. In some cases, the guide strand comprises a phosphorothioate internucleotide linkage between the nucleotides 9 and 10 from the 3′-end of the guide strand. In some cases, the guide strand comprises phosphorothioate internucleotide linkages between the nucleotides at positions 5 and 6 from the 5′-end of the guide strand and between the nucleotides 6 and 7 from the 3′-end of the guide strand. In some cases, the guide strand comprises phosphorothioate internucleotide linkages between the nucleotides at positions 5 and 6 from the 5′-end of the guide strand and between the nucleotides 9 and 10 from the 3′-end of the guide strand. In some cases, the guide strand comprises phosphorothioate internucleotide linkages between the nucleotides at positions 1, 2, 3, and between 5 and 6 from the 5′-end of the guide strand, and between 1, 2, and 3 from the 3′-end of the guide strand. In some cases, the guide strand comprises phosphorothioate internucleotide linkages between the nucleotides at positions 1, 2, 3, and between 5 and 6 from the 5′-end of the guide strand and between the nucleotides at positions 1, 2, and 3, and between 9 and 10 from the 3′-end of the guide strand. In some cases, the guide strand comprises phosphorothioate internucleotide linkages between the nucleotides at positions 1, 2, 3, and between 5 and 6 from the 5′-end of the guide strand and between the nucleotides at positions 1, 2, and 3, and between 6 and 7 from the 3′-end of the guide strand.

In some aspects, the modified nucleotide comprises a modified guanine (e.g., inosine) or one or more of any types of unnatural nucleic acids.

In some aspects, the modified backbone is phosphorothioate, and the phosphorothioate is a stereochemically enriched phosphorothioate. In certain aspects, the strand contains at least one stereochemically enriched phosphorothioate. In some aspects, the strand comprises at least 1, 2, 3 stereochemically enriched phosphorothioates. In some aspects, the strand comprises only 1, 2, 3, or 4 stereochemically enriched phosphorothioates. In further aspects, at least one (e.g., one or two) stereochemically enriched phosphorothioate is disposed between two consecutive nucleosides that are two of six 5′-terminal nucleosides of the strand. In yet further aspects, at least one (e.g., one or two) stereochemically enriched phosphorothioate is disposed between two consecutive nucleosides that are two of six 3′-terminal nucleosides of the strand. In still further aspects, one stereochemically enriched phosphorothioate is covalently bonded to the first nucleoside and the second nucleoside from the 5′-end within the strand. In some aspects, one stereochemically enriched phosphorothioate is covalently bonded to the twenty first nucleoside and the twenty second nucleoside from the 5′-end within the strand. In certain aspects, one stereochemically enriched phosphorothioate is covalently bonded to the twenty second nucleoside and the twenty third nucleoside from the 5′-end within the strand. In particular aspects, the stereochemically enriched phosphorothioate has R_{P} stereochemical identity. In certain aspects, the stereochemically enriched phosphorothioate has S_{P} stereochemical identity.

In some aspects, the polynucleotide molecules described herein comprises one or more (e.g., from 1 to 20, from 1 to 10, or from 1 to 5) stereochemically enriched (e.g., internucleotide) phosphorothioates (e.g., having diastereomeric excess of at least 10%, at least 50%, at least 60%, at least 70%, at least 80%, or at least 90%, e.g., up to about 99%, for the P-stereogenic center). The polynucleotide molecules described herein comprises one or more (e.g., from 1 to 20, from 1 to 10, or from 1 to 5; e.g., internucleoside) phosphorodithioates. The phosphorodithioates may be non-P-stereogenic in the polynucleotide molecules described herein. Phosphorothioates and phosphorodithioates may enhance the stability of the polynucleotide molecules described herein to exonuclease activity of serum. Non-P-stereogenic phosphorodithioates may simplify the synthesis of the polynucleotide molecule described herein by reducing the number of possible diastereomers. Typically, the phosphorothioate or phosphorodithioate may connect two contiguous nucleosides within the six 3′-terminal nucleosides and the six 5′-terminal nucleosides of the polynucleotide molecules described herein. In some aspects, the stereochemically enriched phosphorothioate (e.g., R_{P}-enriched phosphorothioate) may be covalently bonded to the first nucleoside (e.g., the 3′-carbon atom of the first nucleoside) and the second nucleoside (e.g., the 5′-carbon atom of the second nucleoside) from the 5′-end of the antisense strand. Additionally or alternatively, the stereochemically enriched phosphorothioate (e.g., S_{P}-enriched phosphorothioate) may be covalently bonded to the 21^{st} nucleoside (e.g., the 3′-carbon atom of the 21^{st} nucleoside) from the 5′-end and the 22^{nd} nucleoside (e.g., the 5′-carbon atom of the 22^{nd} nucleoside) of the antisense strand. Further, additionally or alternatively, the stereochemically enriched phosphorothioate (e.g., S_{P}-enriched phosphorothioate or Rp-enriched phosphorothioate) may be covalently bonded to the 22^{nd} nucleoside (e.g., the 3′-carbon atom of the 22^{nd} nucleoside) and the 23^{rd} nucleoside (e.g., the 5′-carbon atom of the 23^{rd} nucleoside) from the 5′-end of the antisense strand.

Combinations of a 5′ R_{P}-enriched phosphorothioate (e.g., R_{P}-enriched phosphorothioate covalently bonded to the first nucleoside (e.g., the 3′-carbon atom of the first nucleoside) and the second nucleoside (e.g., the 5′-carbon atom of the second nucleoside) from the 5′-end and a 3′ S_{P}-enriched phosphorothioate (e.g., S_{P}-enriched phosphorothioate covalently bonded to the 21^{st} nucleoside (e.g., the 3′-carbon atom of the 21^{st} nucleoside) and the 22^{nd} nucleoside (e.g., the 5′-carbon atom of the 22^{nd} nucleoside) from the 5′-end in an antisense strand can produce superior efficacy and/or duration of action, e.g., as measured by the reduction in the activity of the target relative to a reference guide strand that lacks the combination of a 5′ R_{P}-enriched phosphorothioate and a 3′ S_{P}-enriched phosphorothioate, or a 5′ Rp-enriched phosphorothioate and a 3′ Sp and Rp-enriched phosphorothioate. In some case, the stereochemically enriched phosphorothioate may comprise RₚRₚSₚSₚ (RₚRₚ at the positions 1 and 2 of the guide strand and SₚSₚ at the positions 21 and 22 of the guide strand) or RₚRₚSₚRₚ(RₚRₚ, at the positions 1 and 2 of the guide strand and SₚRₚ at the positions 21 and 22 of the guide strand). In some aspects, the polynucleotide molecules described herein comprises four stereochemically enriched phosphorothioates: (1) a Rp-enriched phosphorothioate covalently bonded to the 1^{st} nucleoside (e.g., the 3′-carbon atom of the 1^{st} nucleoside) and the 2^{nd} nucleoside (e.g., the 5′-carbon atom of the 2^{nd} nucleoside) from the 5′-end of the antisense strand; (2) a Rp-enriched phosphorothioate covalently bonded to the 22^{nd} nucleoside (e.g., the 3′-carbon atom of the 2^{nd} nucleoside) and the 3^{rd} nucleoside (e.g., the 5′-carbon atom of the 3^{rd} nucleoside) from the 5′-end of the antisense strand; (3) a Sp-enriched phosphorothioate covalently bonded to the 21^{st} nucleoside (e.g., the 3′-carbon atom of the 21^{st} nucleoside) and the 22^{th} nucleoside (e.g., the 5′-carbon atom of the 22^{th} nucleoside) from the 5′-end of the antisense strand; and (4) a Sp-enriched phosphorothioate covalently bonded to the 22^{th} nucleoside (e.g., the 3′-carbon atom of the 22^{th} nucleoside) and the 23^{rd} nucleoside (e.g., the 5′-carbon atom of the 23^{rd} nucleoside) from the 5′-end of the antisense strand. In some aspects, the polynucleotide molecules described herein comprises four stereochemically enriched phosphorothioates: (1) a Rp-enriched phosphorothioate covalently bonded to the 1^{st} nucleoside (e.g., the 3′-carbon atom of the 1^{st} nucleoside) and the 2^{nd} nucleoside (e.g., the 5′-carbon atom of the 2^{nd} nucleoside) from the 5′-end of the antisense strand; (2) a Rp-enriched phosphorothioate covalently bonded to the 2^{nd} nucleoside (e.g., the 3′-carbon atom of the 2^{nd} nucleoside) and the 3^{rd} nucleoside (e.g., the 5′-carbon atom of the 3^{rd} nucleoside) from the 5′-end of the antisense strand; (3) a Sp-enriched phosphorothioate covalently bonded to the 21^{st} nucleoside (e.g., the 3′-carbon atom of the 21^{st} nucleoside) and the 22^{th} nucleoside (e.g., the 5′-carbon atom of the 22^{th} nucleoside) from the 5′-end of the antisense strand; and (4) a Rp-enriched phosphorothioate covalently bonded to the 22^{th} nucleoside (e.g., the 3′-carbon atom of the 22^{th} nucleoside) and the 23^{rd} nucleoside (e.g., the 5′-carbon atom of the 23^{rd} nucleoside) from the 5′-end of the antisense strand.

In some aspects, the stereochemically enriched phosphorothioate internucleotide linkages in the polynucleic acid comprises RₚRₚRₚSₚ (RₚRₚ at the positions 1 and 2 of the guide strand and Sₚ at the positions 21 of the guide strand) or RₚRₚRₚSₚ (RₚRₚ at the positions 1, 2, 3 of the guide strand and Sₚ at the positions 21 of the guide strand).

In some aspects, the polynucleotide molecule described herein comprises one or more purine modification. In some aspects, the purine modification described herein is 2,6-diaminopurine. In some aspects, the purine modification described herein is 3-deaza-adenine. In some aspects, the purine modification described herein is 7-deaza-guanine. In some aspects, the purine modification described herein is 8-azido-adenine.

In some aspects, the polynucleotide molecule described herein comprises one or more pyrimidine modification. In some aspects, the pyrimidine modification described herein is 2-thio-thymidine. In some aspects, the pyrimidine modification described herein is 5-carboxamide-uracil. In some aspects, the pyrimidine modification described herein is 5-methyl-cytosine. In some aspects, the pyrimidine modification described herein is 5-ethynyl uracil.

In some cases, the polynucleic acid molecule described herein comprises an abasic substitution. In those cases where a hybridized polynucleotide construct is contemplated for use as siRNA, a reduction of miRNA-like off-target effects is desirable. The inclusion of one or more (e.g., one or two) abasic substitutions in the hybridized polynucleotide constructs may reduce or even eliminate miRNA-like off-target effects, as the abasic substitutions lack nucleobases that are capable of engaging in base-pairing interactions and alleviate steric hindrance. Thus, the polynucleotide molecule disclosed herein may include one or more (e.g., one or two) abasic substitutions. In some aspects, abasic substitution is at the 5^{th} nucleotide from the 5′ end of the antisense strand described herein. In some aspects, abasic substitution is at the 7^{th} nucleotide from the 5′ end of the antisense strand described herein.

When the polynucleotide molecule disclosed herein includes two or more of the abasic substitutions, their structures may be same or different. In certain aspects, a sense strand contains one abasic substitution (e.g., an antisense strand may be free of abasic substitutions). In other aspects, an antisense strand contains one abasic substitution (e.g., a sense strand may be free of abasic substitutions). In yet other aspects, an antisense strand contains one abasic substitution, and a sense strand contains one abasic substitution. In further aspects, a sense strand includes an abasic substitution between a nucleoside number (x) and a nucleoside number (x+1), where x is an integer from 2 to 7. In yet further aspects, an antisense strand includes an abasic substitution between a nucleoside number (x) and a nucleoside number (x+1), where x is an integer from 2 to 7.

The abasic substitution may be or formula (III):

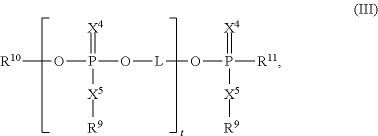

where (III)
L is a sugar analogue, or is substituted with a heteroacyl from A, U, C, G, or is any other substituted nucleic acid (e.g., locked or unlocked nucleic acid, glycol nucleic acid, etc.);
each X⁴ is independently O or S;
each X⁵ is independently O, S, NH, or a bond;
each R⁹ is independently H, optionally substituted C₁₋₆ alkyl, optionally substituted C₂₋₆ alkenyl, optionally substituted C₂₋₆ alkynyl, optionally substituted (C₁₋₉ heterocyclyl)-C₁₋₆-alkyl, optionally substituted (C₆₋₁₀ aryl)-C₁₋₆-alkyl, optionally substituted (C₃₋₈ cycloalkyl)-C₁₋₆-alkyl, -LinkA(-T)ₚ, or a conjugation moiety;
each LinkA is independently a multivalent linker (e.g., including —C(O)—N(H)—);
each T is independently an auxiliary moiety;
R¹⁰ is a bond to a 3′-carbon atom of a nucleoside (x) in the strand;
R¹¹ is a bond to a 5′-oxygen atom of a nucleoside (x+1) in the strand;
p is an integer from 1 to 6; and
t is an integer from 1 to 6.

In some aspects, the abasic substitution described herein is attached to the antisense strand of the polynucleic acid molecule described herein. In particular aspects, an abasic substitution (e.g., an internucleotide, abasic spacer of formula (III) in which t is 1) may be included in the antisense strand described herein (e.g., within the seed region of the guide strand). In some aspects, an abasic substitution (e.g., an internucleotide, abasic spacer of formula (III) in which t is 1) may be bonded to the 3′ carbon atom of the second, third, fourth, or fifth nucleoside from the 5′-end of the antisense strand described herein. In certain aspects, an abasic substitution (e.g., an internucleotide, abasic spacer of formula (III) in which t is 1) may be bonded to the 3′ carbon atom of the thirteenth, fourteenth, fifteenth, or sixteenth nucleoside from the 5′-end of the antisense strand described herein. In some aspects, an abasic substitution fourth, fifth, sixth, seventh, eighth, and/or ninth nucleoside from the 5′-end of the antisense strand described herein.

The polynucleotide molecule described herein may contain a strand including a seed region including a hypoxanthine nucleobase-containing nucleoside (e.g., inosine).

In certain aspects, the hypoxanthine nucleobase-containing nucleoside is a second nucleoside from the 5′-end in the strand. In further aspects, the hypoxanthine nucleobase-containing nucleoside is a third nucleoside from the 5′-end in the strand. In yet further aspects, the hypoxanthine nucleobase-containing nucleoside is a fourth nucleoside from the 5′-end in the strand. In still further aspects, the hypoxanthine nucleobase-containing nucleoside is a fifth nucleoside from the 5′-end in the strand. In particular aspects, the hypoxanthine nucleobase-containing nucleoside is a sixth nucleoside in the strand. In particular aspects, the hypoxanthine nucleobase-containing nucleoside is a seventh nucleoside in the strand.

In some instances, the polynucleotide molecule described herein comprises at least one inosine substitution. In some cases, the at least one inosine substitution is within a seed region of the antisense strand. In some cases, the at least one inosine substitution is within 7 nucleotides from the 5′ end of the antisense strand. In some cases, the at least one inosine substitution is in the first nucleotide from the 5′ end of the antisense strand (e.g., SRS-002331). In some cases, the at least one inosine substitution comprises 2′-O-methylinosine-3′-phosphate.

In some aspects, the polynucleotide molecule described herein comprises a sense strand and an antisense strand, and the first nucleotide from the 5′ end of the antisense strand is substituted by a uridine and the last nucleotide from the 3′ end of the sense strand is substituted by an adenosine (e.g., SRS-002376). In some cases, the uridine and/or the adenosine is modified. In some cases, the uridine comprises 2′-O-methyluridine-3′-phosphate and the adenosine comprises 2′-O-methyladenosine-3′-phosphate. In some cases, the uridine comprises 2′-O-methyluridine-3′-phosphate or the adenosine comprises 2′-O-methyladenosine-3′-phosphate.

In some aspects, the first nucleotide from the 5′ end of the antisense strand of the polynucleotide molecule described herein is substituted by an adenosine. In some cases, the adenosine is a modified adenosine. In some cases, the adenosine comprises 2′-O-methyl-8-bromo-adenosine-3′-phopshate.

### The Amount and Location of Modifications

In some aspects, the polynucleotide molecule described herein comprises one or more type of modifications as described above. Accordingly, in some aspects, about 10% of the nucleotides from the polynucleotide molecule described herein are modified with one or more type of modifications as described above. In other aspects, about 20% of the nucleotides from the polynucleotide molecule described herein are modified with one or more type of modifications as described above. In other aspects, about 30% of the nucleotides from the polynucleotide molecule described herein are modified with one or more type of modifications as described above. In other aspects, about 40% of the nucleotides from the polynucleotide molecule described herein are modified with one or more type of modifications as described above. In other aspects, about 50% of the nucleotides from the polynucleotide molecule described herein are modified with one or more type of modifications as described above. In other aspects, about 60% of the nucleotides from the polynucleotide molecule described herein are modified with one or more type of modifications as described above. In other aspects, about 70% of the nucleotides from the polynucleotide molecule described herein are modified with one or more type of modifications as described above. In other aspects, about 80% of the nucleotides from the polynucleotide molecule described herein are modified with one or more type of modifications as described above. In other aspects, about 90% of the nucleotides from the polynucleotide molecule described herein are modified with one or more type of modifications as described above. In other aspects, 100% of the nucleotides from the polynucleotide molecule described herein are modified with one or more type of modifications as described above.

In some aspects, the one or more types of modifications described herein occurs at different positions within the polynucleotide molecule described herein. In some aspects, the one or more types of modifications described herein occurs in the seed region within the polynucleotide molecule described herein. In some aspects, the one or more types of modifications described herein occurs at 3′ terminal of the polynucleotide molecule described herein. In some aspects, the one or more types of modifications described herein occurs at 5′ terminal of the polynucleotide molecule described herein. In some aspects, the one or more types of modifications described herein occurs dispersedly within the polynucleotide molecule described herein. In some aspects, the one or more types of modifications described herein occurs in clusters within the polynucleotide molecule described herein.

### Specific Modification Patterns

In some aspects, described herein is a specific modification pattern for the polynucleic acid molecule which is a double-stranded nucleic acid molecule comprising a sense strand and an antisense strand. In some aspects, the antisense strand comprises a 2′-fluoro modified nucleotide in position 2 from the 5′ end. In some aspects, the antisense strand comprises a 2′-fluoro modified nucleotide in position 14 from the 5′ end. In some aspects, the antisense strand comprises 2′-fluoro modified nucleotides in positions 2 and 14 from the 5′ end. In some aspects, the antisense strand comprises a 2′-fluoro modified nucleotide in position 12 from the 5′ end. In some aspects, the antisense strand comprises a 2′-fluoro modified nucleotide in position 16 from the 5′ end. In other aspects, the antisense strand comprises a 2′-fluoro modified nucleotide in position 6 from the 5′ end. In other aspects, the antisense strand comprises a 2′-fluoro modified nucleotide in position 7 from the 5′ end. In other aspects, the antisense strand comprises a 2′-fluoro modified nucleotide in position 8 from the 5′ end. In other aspects, the antisense strand comprises a 2′-fluoro modified nucleotide in position 9 from the 5′ end. In other aspects, the antisense strand comprises a 2′-fluoro modified nucleotide in position 4 from the 5′ end.

In some aspects, described herein is a specific modification pattern for the polynucleic acid molecule which is a double-stranded nucleic acid molecule comprising a sense strand and an antisense strand. In some aspects, the sense strand comprises a 2′-fluoro modified nucleotide in position 9 from the 5′ end. In some aspects, the sense strand comprises a 2′-fluoro modified nucleotide in position 11 from the 5′ end. In some aspects, the sense strand comprises 2′-fluoro modified nucleotides in positions 9 and 11 from the 5′ end. In some aspects, the sense strand comprises a 2′-fluoro modified nucleotide in position 7 from the 5′ end. In some aspects, the sense strand comprises a 2′-fluoro modified nucleotide in position 10 from the 5′ end. In some aspects, the sense strand comprises 2′-fluoro modified nucleotides in positions 9, 11, and 7 from the 5′ end, the sense strand comprises 2′-fluoro modified nucleotides in positions 9 and 11, and 10 from the 5′ end, the sense strand comprises 2′-fluoro modified nucleotides in positions 9 and 7 from the 5′ end, the sense strand comprises 2′-fluoro modified nucleotides in positions 9 and 10 from the 5′ end. the sense strand comprises 2′-fluoro modified nucleotides in positions 9, 11, 7, and 10 from the 5′ end. In other aspects, the sense strand comprises a 2′-fluoro modified nucleotide in position 8 from the 5′ end. In other aspects, the sense strand comprises a 2′-fluoro modified nucleotide in position 12 from the 5′ end. In other aspects, the sense strand comprises a 2′-fluoro modified nucleotide in position 16 from the 5′ end.

In some aspects, the sense and antisense strand of the polynucleic acid molecule comprises any combination of two or more 2′-fluoro modified nucleotides at the positions described in the above two paragraphs.

In some aspects, the antisense strand comprises 5′-nNfnnnNfnNfNfnnnnNfnNfnnnnnnn-3′. In some aspects, the antisense strand comprises 5′-nNfnnnNfnnnnnnnNfnNfnnnnnnn-3′. In some aspects, the antisense strand comprises 5′-nNfnnnnNfnnnnNfnNfnnnnnnnnn-3′. In the modification patterns described above, “Nf” stands for a 2′-fluoro modified nucleotide, and wherein “n” stands for a 2′-O-methyl modified nucleotide.

In some aspects, the sense strand comprises 5′-nnnnnnNfnNfnNfnnnnnnnnnn-3′. In some aspects, the sense strand comprises 5′-nnnnnnNfnNfNfNfnnnnnnnnnn-3′. In some aspects, the sense strand comprises 5′-nnnnnnnnNfNfNfnnnnnnnnnn-3′. In the modification patterns described above, “Nf” stands for a 2′-fluoro modified nucleotide, and wherein “n” stands for a 2′-O-methyl modified nucleotide.

In some aspects, described herein is a specific modification pattern for the polynucleic acid molecule which is a double-stranded nucleic acid molecule comprising a sense strand and an antisense strand, wherein the sense strand comprises about twelve 2′-fluoro modified nucleotides and about nine 2′-O-methyl modified nucleotides, and wherein the antisense strand comprises about nine 2′-fluoro modified nucleotides and about fourteen 2′-O-methyl modified nucleotides.

In some aspects, described herein is a specific modification pattern, wherein the sense strand is fully modified and comprises twelve 2′-fluoro modified nucleotides, nine 2′-O-methyl modified nucleotides, and wherein the antisense strand is fully modified and comprises nine 2′-fluoro modified nucleotides and fourteen 2′-O-methyl modified nucleotides.

In some aspects, described herein is a specific modification pattern, wherein the sense strand comprises 5′-NfnNfnNfnNfnNfNfNfnNfnNfnNfnNfnNf-3′, wherein the antisense strand comprises 5′-nNfnNfnNfnNfnNfnnnNfnNfnNfnNfnnn-3′, wherein “Nf” stands for a 2′-fluoro modified nucleotide, and wherein “n” stands for a 2′-O-methyl modified nucleotide.

In some aspects, described herein is a specific modification pattern, wherein the sense strand comprises 5′-NfnNfnNfnNfnNfNfNfnNfnNfnNfnNfnNf-3′, wherein the antisense strand comprises 5′-nNfnNfnNfnNfnNfnnnNfnNfnNfnNfnnn-3′, wherein the sense and/or antisense strand comprises one or more phosphorothioate linkage, wherein ‘Nf’ stands for a 2′-fluoro modified nucleotide, and wherein “n” stands for a 2′-O-methyl modified nucleotide. In other aspects, described herein is a specific modification pattern, wherein the sense strand comprises 5′-NfnNfnNfnNfnNfNfNfnNfnNfnNfnNfnNf-3′, wherein the antisense strand comprises 5′-nNfnNfnNfnNfnNfnnnNfnNfnNfnNfnnn-3′, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type I in Table 11.

TABLE 11
Nucleotide Modification Patterns

| Pattern Name | Pattern |
|---|---|
| Type I for sense strand | 5′-NfsnsNfnNfnNfnNfNfNfnNfnNfnNfnNfnNf-3′ |
| Type I for antisense strand | 5′-nsNfsnNfnNfnNfnNfnnnNfnNfnNfnNfnsnsn-3′ |
| Type II for sense strand | 5′-nsnsnnnnNfnNfNfNfnnnnnnnnnn-3′ |
| Type II for antisense strand | 5′-nsNfsnnnNfnNfNfnnnnNfnNfnnnnnsnsn-3′ |
| Type III for sense strand | 5′-nsnsnnnnnnNfnNfnnnnnnnnnn-3′ |
| Type III for antisense strand | 5′-nsNfsnnnnnnnnnNfnNfnnnnnnnsnsn-3′ |
| Type IV for sense strand | 5′-nsnsnnnnNfnNfnNfnnnnnnnnnn-3′ |
| Type IV for antisense strand | 5′-nsNfsnnnnnnnnnNfnNfnNfnnnnnsnsn-3′ |
| Type V for sense strand | 5′-nsnsnnnnNfnNfnNfnnnnnnnnnn-3′ |
| Type V for antisense strand | 5′-nsNfsnnnnNfnnnnNfnNfnNfnnnnnsnsn-3′ |
| Type VI for sense strand | 5′-nnnnnnNfnNfnNfnnnnnnnnnn-invdN-invdN-3′ |

Note:
“Nf” stands for a 2′-fluoro modified nucleotide, ″“n” stands for a 2′-O-methyl modified nucleotide, “s” stands for a 3′-phosphorothioate, “invdN” stands for an inverted deoxy-nucleotide.

In some aspects, the polynucleotide molecule provided herein comprises a sense strand comprising a nucleic acid sequence of SEQ ID NOs: 101-150 and 220 and an antisense strand comprises a nucleic acid sequence of SEQ ID NOs: 1-50 and 215-216. In other aspects, the polynucleotide molecule provided herein comprises a sense strand comprising a nucleic acid sequence of SEQ ID NOs: 101-150 and 220, an antisense strand comprises a nucleic acid sequence of SEQ ID NOs: 1-50 and 215-216, and wherein the sense and/or antisense strand is modified in Type I modification pattern specified in Table 11. In some aspects, the polynucleotide molecule provided herein comprises a sense strand comprising a nucleic acid sequence of SEQ ID NOs: 104, 106-107, 109-111, 113-114, 135, 150, and 220 and an antisense strand comprises a nucleic acid sequence of SEQ ID NOs: 4, 6-7, 9-11, 13-14, 35, 50, and 215-216. In other aspects, the polynucleotide molecule provided herein comprises a sense strand comprising a nucleic acid sequence of SEQ ID NOs: 104, 106-107, 109-111, 113-114, 135, 150, and 220, an antisense strand comprises a nucleic acid sequence of SEQ ID NOs: 4, 6-7, 9-11, 13-14, 35, 50, and 215-216, and wherein the sense and/or anti sense strand is modified in Type I modification pattern specified in Table 11.

In some aspects, described herein is a specific modification pattern, wherein the sense strand comprises about four 2′-fluoro modified nucleotides and about seventeen 2′-O-methyl modified nucleotides, and wherein the antisense strand comprises about six 2′-fluoro modified nucleotides and about seventeen 2′-O-methyl modified nucleotides.

In some aspects, described herein is a specific modification pattern, wherein the sense strand is fully modified and comprises four 2′-fluoro modified nucleotides, seventeen 2′-O-methyl modified nucleotides, and wherein the antisense strand is fully modified and comprises six 2′-fluoro modified nucleotides and seventeen 2′-O-methyl modified nucleotides.

In some aspects, described herein is a specific modification pattern, wherein the sense strand comprises 5′-nnnnnnNfnNfNfNfnnnnnnnnnn-3′, wherein the antisense strand comprises 5′-nNfnnnNfnNfNfnnnnNfnNfnnnnnnn-3′, wherein “Nf” stands for a 2′-fluoro modified nucleotide, and wherein “n” stands for a 2′-O-methyl modified nucleotide.

In some aspects, described herein is a specific modification pattern, wherein the sense strand comprises 5′-nnnnnnNfnNfNfNfnnnnnnnnnn-3′, wherein the antisense strand comprises 5′-nNfnnnNfnNfNfnnnnNfnNfnnnnnnn-3′, wherein the sense and/or antisense strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the sense strand comprises 5′-nnnnnnNfnNfNfNfnnnnnnnnnn-3′, wherein the antisense strand comprises 5′-nNfnnnNfnNfNfnnnnNfnNfnnnnnnn-3′, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type II in Table 11.

In some aspects, the polynucleotide molecule provided herein comprises a sense strand comprising a nucleic acid sequence of SEQ ID NOs: 101-150 and 220, and/or an antisense strand comprising a nucleic acid sequence of SEQ ID NOs: 1-50 and 215-216, and wherein the sense and/or antisense strand is modified in Type II modification pattern specified in Table 11. In other aspects, the polynucleotide molecule provided herein comprises a sense strand comprising a nucleic acid sequence of SEQ ID NOs: 104, 106-107, 109-111, 113-114, 135, 150, and 220, and/or an antisense strand comprising a nucleic acid sequence of SEQ ID NOs: 4, 6-7, 9-11, 13-14, 35, 50, and 215-216, and wherein the sense and/or antisense strand is modified in Type II modification pattern specified in Table 11.

In some aspects, described herein is a specific modification pattern, wherein the sense strand comprises about two 2′-fluoro modified nucleotides and about nineteen 2′-O-methyl modified nucleotides, and wherein the antisense strand comprises about three 2′-fluoro modified nucleotides and about twenty 2′-O-methyl modified nucleotides.

In some aspects, described herein is a specific modification pattern, wherein the sense strand is fully modified and comprises two 2′-fluoro modified nucleotides and nineteen 2′-O-methyl modified nucleotides, and wherein the antisense strand is fully modified and comprises three 2′-fluoro modified nucleotides and twenty 2′-O-methyl modified nucleotides.

In some aspects, described herein is a specific modification pattern, wherein the sense strand comprises 5′-nnnnnnnnNfnNfnnnnnnnnnn-3′, wherein the antisense strand comprises 5′-nNfnnnnnnnnnNfnNfnnnnnnnnn-3′, wherein “Nf” stands for a 2′-fluoro modified nucleotide, and wherein “n” stands for a 2′-O-methyl modified nucleotide.

In some aspects, described herein is a specific modification pattern, wherein the sense strand comprises 5′-nnnnnnnnNfnNfnnnnnnnnnn-3′, wherein the antisense strand comprises 5′-nNfnnnnnnnnnNfnNfnnnnnnnnn-3′, wherein the sense and/or antisense strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the sense strand comprises 5′-nnnnnnnnNfnNfnnnnnnnnnn-3′, wherein the antisense strand comprises 5′-nNfnnnnnnnnnNfnNfnnnnnnnnn-3′, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type III in Table 11.

In some aspects, the polynucleotide molecule provided herein comprises a sense strand comprising a nucleic acid sequence of SEQ ID NOs: 101-150 and 220, and/or an antisense strand comprising a nucleic acid sequence of SEQ ID NOs: 1-50 and 215-216, and wherein the sense and/or antisense strand is modified in Type III modification pattern specified in Table 11. In other aspects, the polynucleotide molecule provided herein comprises a sense strand comprising a nucleic acid sequence of SEQ ID NOs: 104, 106-107, 109-111, 113-114, 135, 150, and 220, and/or an antisense strand comprising a nucleic acid sequence of SEQ ID NOs: 4, 6-7, 9-11, 13-14, 35, 50, and 215-216, and wherein the sense and/or antisense strand is modified in Type Ill modification pattern specified in Table 11.

In some aspects, described herein is a specific modification pattern, wherein the sense strand comprises about three 2′-fluoro modified nucleotides and about eighteen 2′-O-methyl modified nucleotides, and wherein the antisense strand comprises about four 2′-fluoro modified nucleotides and about nineteen 2′-O-methyl modified nucleotides.

In some aspects, described herein is a specific modification pattern, wherein the sense strand is fully modified and comprises three 2′-fluoro modified nucleotides, eighteen 2′-O-methyl modified nucleotides, and wherein the antisense strand is fully modified and comprises four 2′-fluoro modified nucleotides, nineteen 2′-O-methyl modified nucleotides.

In some aspects, described herein is a specific modification pattern, wherein the sense strand comprises 5′-nnnnnnNfnNfnNfnnnnnnnnnn-3′, wherein the antisense strand comprises 5′-nNfnnnnnnnnnNfnNfnNfnnnnnnn-3′, wherein “Nf” stands for a 2′-fluoro modified nucleotide, and wherein “n” stands for a 2′-O-methyl modified nucleotide.

In some aspects, described herein is a specific modification pattern, wherein the sense strand comprises 5′-nnnnnnNfnNfnNfnnnnnnnnnn-3′, wherein the antisense strand comprises 5′-nNfnnnnnnnnnNfnNfnNfnnnnnnn-3′, wherein the sense and/or antisense strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the sense strand comprises 5′-nnnnnnNfnNfnNfnnnnnnnnnn-3′, wherein the antisense strand comprises 5′-nNfnnnnnnnnnNfnNfnNfnnnnnnn-3′, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type IV in Table 11.

In some aspects, the polynucleotide molecule provided herein comprises a sense strand comprising a nucleic acid sequence of SEQ ID NOs: 101-150 and 220, and/or an antisense strand comprising a nucleic acid sequence of SEQ ID NOs: 1-50 and 215-216, and wherein the sense and/or antisense strand is modified in Type IV modification pattern specified in Table 11. In other aspects, the polynucleotide molecule provided herein comprises a sense strand comprising a nucleic acid sequence of SEQ ID NOs: 104, 106-107, 109-111, 113-114, 135, 150, and 220, and/or an antisense strand comprising a nucleic acid sequence of SEQ ID NOs: 4, 6-7, 9-11, 13-14, 35, 50, and 215-216, and wherein the sense and/or antisense strand is modified in Type IV modification pattern specified in Table 11.

In some aspects, described herein is a specific modification pattern, wherein the sense strand comprises about three 2′-fluoro modified nucleotides and about eighteen 2′-O-methyl modified nucleotides, and wherein the antisense strand comprises about five 2′-fluoro modified nucleotides and about eighteen 2′-O-methyl modified nucleotides.

In some aspects, described herein is a specific modification pattern, wherein the sense strand is fully modified and comprises three 2′-fluoro modified nucleotides and eighteen 2′-O-methyl modified nucleotides, and wherein the antisense strand is fully modified and comprises five 2′-fluoro modified nucleotides, eighteen 2′-O-methyl modified nucleotides.

In some aspects, described herein is a specific modification pattern, wherein the sense strand comprises 5′-nnnnnnNfnNfnNfnnnnnnnnnn-3′, wherein the antisense strand comprises 5′-nNfnnnnNfnnnnNfnNfnNfnnnnnnn-3′, wherein “Nf” stands for a 2′-fluoro modified nucleotide, and wherein “n” stands for a 2′-O-methyl modified nucleotide.

In some aspects, described herein is a specific modification pattern, wherein the sense strand comprises 5′-nnnnnnNfnNfnNfnnnnnnnnnn-3′, wherein the antisense strand comprises 5′-nNfnnnnNfnnnnNfnNfnNfnnnnnnn-3′, wherein the sense and/or antisense strand comprises one or more phosphorothioate linkage, wherein “Nf” stands for a 2′-fluoro modified nucleotide, and wherein “n” stands for a 2′-O-methyl modified nucleotide. In other aspects, described herein is a specific modification pattern, wherein the sense strand comprises 5′-nnnnnnNfnNfnNfnnnnnnnnnn-3′, wherein the antisense strand comprises 5′-nNfnnnnNfnnnnNfnNfnNfnnnnnnn-3′, wherein the sense comprises two phosphorothioate linkages, wherein the antisense comprises four phosphorothioate linkages, wherein “Nf” stands for a 2′-fluoro modified nucleotide, and wherein “n” stands for a 2′-O-methyl modified nucleotide.

In some aspects, described herein is a specific modification pattern, wherein the sense strand and/or antisense strand is modified as Type V in Table 11.

In some aspects, the polynucleotide molecule provided herein comprises a sense strand comprising a nucleic acid sequence of SEQ ID NOs: 101-150 and 220, and/or an antisense strand comprising a nucleic acid sequence of SEQ ID NOs: 1-50 and 215-216, and wherein the sense and/or antisense strand is modified in Type V modification pattern specified in Table 11. In other aspects, the polynucleotide molecule provided herein comprises a sense strand comprising a nucleic acid sequence of SEQ ID NOs: 104, 106-107, 109-111, 113-114, 135, 150, and 220, and/or an antisense strand comprising a nucleic acid sequence of SEQ ID NOs: 4, 6-7, 9-11, 13-14, 35, 50, and 215-216, and wherein the sense and/or antisense strand is modified in Type V modification pattern specified in Table 11.

In some aspects, described herein is a specific modification pattern, wherein the sense strand comprises about three 2′-fluoro modified nucleotides and about eighteen 2′-O-methyl modified nucleotides, with one or more inverted deoxy-nucleotides on the 3′ end as an overhang.

In some aspects, described herein is a specific modification pattern, wherein the sense strand is fully modified and comprises three 2′-fluoro modified nucleotides and eighteen 2′-O-methyl modified nucleotides, with two inverted deoxy-nucleotides on the 3′ end as an overhang.

In some aspects, described herein is a specific modification pattern, wherein the sense strand comprises 5′-nnnnnnNfnNfnNfnnnnnnnnnn-invdN-invdN-3′, wherein “Nf” stands for a 2′-fluoro modified nucleotide, wherein “n” stands for a 2′-O-methyl modified nucleotide, and “invdN” stands for an inverted deoxy-nucleotide. In some instances, the invdN is an inverted deoxyl-thymine. In some aspects, the linker conjugated with one or more targeting moieties as shown in Formula (IV″) or (IV′″) is added to the first nucleic acid on the 5′ end. In some aspects, the linker conjugated with one or more GalNAc as shown in Formula (V″) or (V′″) is added to the first nucleic acid on the 5′ end. In some aspects, the modification pattern comprises one or more phosphorothioate linkages. In some aspects, the modification pattern is shown in Formula (VII). In some aspects, the 5′ end modification known in the art is applied to the one or more inverted nucleotides.

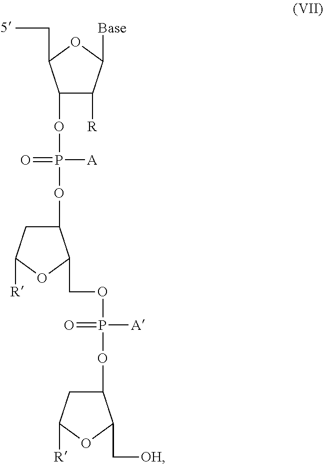

wherein R is a moiety that corresponds to the sugar modification described herein, in some instances, R is —O-methyl; wherein R′ is thymine, abasic, or others; wherein A is —O or —S; and wherein A′ is —O or —S.

In some aspects, the polynucleotide molecule provided herein comprises a sense strand comprising a nucleic acid sequence selected from SEQ ID NOs: 101-150 and 220, and/or an antisense strand comprising a nucleic acid sequence selected from SEQ ID NOs: 1-50 and 215-216, and wherein the sense strand is modified in Type VI modification pattern specified in Table 11 or as described in the preceding paragraph. In other aspects, the polynucleotide molecule provided herein comprises a sense strand comprising a nucleic acid sequence selected from SEQ ID NOs: 104, 106-107, 109-111, 113-114, 135, 150, and 220, and/or an antisense strand comprising a nucleic acid sequence selected from SEQ ID NOs: 4, 6-7, 9-11, 13-14, 35, 50, and 215-216, and wherein the sense strand is modified in Type VI modification pattern specified in Table 11 or as described in the preceding paragraph.

Described herein is a polynucleic acid molecule, whose sense strand comprises a nucleic acid sequence that is at least 80% identical to a nucleic acid sequence selected from a nucleic acid sequence selected from SEQ ID NOs: 151-200, 214, and 221. Described herein is a polynucleic acid molecule, whose sense strand comprises a nucleic acid sequence that is at least 85% identical to a nucleic acid sequence selected from a nucleic acid sequence selected from SEQ ID NOs: 151-200, 214, and 221. Described herein is a polynucleic acid molecule, whose sense strand comprises a nucleic acid sequence that is at least 90% identical to a nucleic acid sequence selected from SEQ ID NOs: 151-200, 214, and 221. Described herein is a polynucleic acid molecule, whose sense strand comprises a nucleic acid sequence that is at least 95% identical to a nucleic acid sequence selected from SEQ ID NOs: 151-200, 214, and 221.

Described herein is a polynucleic acid molecule, which antisense strand comprises a nucleic acid sequence that is at least 80% identical to a nucleic acid sequence selected from SEQ ID NOs: 51-100, 201-213, and 217-219. Described herein is a polynucleic acid molecule, which antisense strand comprises a nucleic acid sequence that is at least 85% identical to a nucleic acid sequence selected from SEQ ID NOs: 51-100, 201-213, and 217-219. Described herein is a polynucleic acid molecule, which antisense strand comprises a nucleic acid sequence that is at least 90% identical to a nucleic acid sequence selected from SEQ ID NOs: 51-100, 201-213, and 217-219. Described herein is a polynucleic acid molecule, which antisense strand comprises a nucleic acid sequence that is at least 95% identical to a nucleic acid sequence selected from SEQ ID NOs: 51-100, 201-213, and 217-219.

A polynucleic acid molecule for modulating expression of FXI gene, wherein polynucleic acid molecule comprises an antisense strand comprising the nucleotide sequence selected from SEQ ID NOs: 4, 6-7, 9-11, 13-14, 35, 50, and 215-216 and a sense strand comprising the nucleotide sequence selected from SEQ ID NOs: 104, 106-107, 109-111, 113-114, 135, 150, and 220.

A poly nucleic acid molecule for modulating expression of FXI gene, wherein polynucleic acid molecule comprises an antisense strand comprising the nucleotide sequence of SEQ ID NO: 11 and a sense strand comprising the nucleotide sequence of SEQ ID NO: 111.

A polynucleic acid molecule for modulating expression of FXI gene, wherein polynucleic acid molecule comprises an antisense strand comprising the nucleotide sequence selected from SEQ ID NOs: 54, 56-57, 59-61, 63-64, 85, 100, 201-213, and 217-219 and a sense strand comprising the nucleotide sequence selected from SEQ ID NOs: 154, 156-157, 159-161, 163-164, 185, 200, 214, and 221.

A poly nucleic acid molecule for modulating expression of FXI gene, wherein polynucleic acid molecule comprises an antisense strand comprising the nucleotide sequence of SEQ ID NO: 61 and a sense strand comprising the nucleotide sequence of SEQ ID NO: 161.

Further provided herein is a polynucleic acid molecule for modulating expression of FXI gene, wherein polynucleic acid molecule comprises an antisense strand comprising the nucleotide sequence of asUfsaaaugucuuUfgUfuGfcaagcsgsc (SEQ ID NO: 61) and a sense strand comprising the nucleotide sequence of gscsuugcAfaCfaAfagacauuuau (SEQ ID NO: 161), wherein “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate, “s” refers to 3′-phosphorothioate, “invdT” refers to 3′ inverted thymidine, “i” refers to 2′-O-methylinosine-3′-phosphate, “a4” refers to 2′-O-methyl-8-bromo-adenosine-3′-phopshate.

Further provided herein is a polynucleic acid molecule for modulating expression of FXI gene, wherein polynucleic acid molecule comprises an antisense strand comprising the nucleotide sequence of asUfsaaaugucuuUfgUfuGfcsaagcsgsc (SEQ ID NO: 211) and a sense strand comprising the nucleotide sequence of gscsuugcAfaCfaAfagacauuuau (SEQ ID NO: 161), wherein “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate, “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “invdT” refers to 3′ inverted thymidine, “i” refers to 2′-O-methylinosine-3′-phosphate, “a4” refers to 2′-O-methyl-8-bromo-adenosine-3′-phopshate.

Further provided herein is a polynucleic acid molecule for modulating expression of FXI gene, wherein polynucleic acid molecule comprises an antisense strand comprising the nucleotide sequence of asUfsaaaugucuuUfgUfsuGfcaagcsgsc (SEQ ID NO: 212) and a sense strand comprising the nucleotide sequence of gscsuugcAfaCfaAfagacauuuau (SEQ ID NO: 161), wherein “A” refers to adenosine-3′-phosphate; “a” refers to 2′-O-methyladenosine-3′-phosphate, “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “d C” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate; “If” refers to 2′-fluoro-5-methyluridine-3′-phosphate; “dT” refers to 2′-deoxythymidine-3′-phosphate, “s” refers to 3′-phosphorothioate, “invdT” refers to 3′ inverted thymidine, “i” refers to 2′-O-methylinosine-3′-phosphate, “a4” refers to 2′-O-methyl-8-bromo-adenosine-3′-phopshate.

Further provided herein is a polynucleic acid molecule for modulating expression of FXI gene, wherein polynucleic acid molecule comprises an antisense strand comprising the nucleotide sequence of asUfsaaaugucuuUfgUfuGfcaasgcsgsc (SEQ ID NO: 213) and a sense strand comprising the nucleotide sequence of gscsuugcAfaCfaAfagacauuuau (SEQ ID NO: 161), wherein “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “invdT” refers to 3′ inverted thymidine, “i” refers to 2′-O-methylinosine-3′-phosphate, “a4” refers to 2′-O-methyl-8-bromo-adenosine-3′-phopshate.

Further provided herein is a polynucleic acid molecule for modulating expression of FXI gene, wherein polynucleic acid molecule comprises an antisense strand comprising the nucleotide sequence of asUfsaaaugucuuUfgUfuGfcaagcsgsc (SEQ ID NO: 61) and a sense strand comprising the nucleotide sequence of gscsuugcAfaCfaAfagacauuuau(invdT)(invdT) (SEQ ID NO: 214), wherein “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “C” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “invdT” refers to 3′ inverted thymidine, “i” refers to 2′-O-methylinosine-3′-phosphate, “a4” refers to 2′-O-methyl-8-bromo-adenosine-3′-phopshate.

Further provided herein is a polynucleic acid molecule for modulating expression of FXI gene, wherein polynucleic acid molecule comprises an antisense strand comprising the nucleotide sequence of asUfsgucuUfuguuGfcAfaGfcgcuusasu (SEQ ID NO: 204) and a sense strand comprising the nucleotide sequence of asasgcgcUfuGfcAfacaaagacau (SEQ ID NO: 159), wherein “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “of” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “C” refers to 2′-O-methyl-5-methyluridine-3′-phosphate; “Tf” refers to 2′-fluoro-5-methyluridine-3′-phosphate; “dl” refers to 2′-deoxythymidine-3′-phosphate; “s” refers to 3′-phosphorothioate, “invdT” refers to 3′ inverted thymidine, “i” refers to 2′-O-methylinosine-3′-phosphate, “a4” refers to 2′-O-methyl-8-bromo-adenosine-3′-phopshate.

Further provided herein is a polynucleic acid molecule for modulating expression of FXI gene, wherein polynucleic acid molecule comprises an antisense strand comprising the nucleotide sequence of asAfsugucUfuuguUfgCfaAfgcgcususa (SEQ ID NO: 205) and a sense strand comprising the nucleotide sequence of asgscgcuUfgCfaAfcaaagacauu (SEQ ID NO: 160), wherein “A” refers to adenosine-3′-phosphate; “a” refers to 2′-O-methyladenosine-3′-phosphate, “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “d C” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate, “s” refers to 3′-phosphorothioate, “invdT” refers to 3′ inverted thymidine, “i” refers to 2′-O-methylinosine-3′-phosphate, “a4” refers to 2′-O-methyl-8-bromo-adenosine-3′-phopshate.

Further provided herein is a polynucleic acid molecule for modulating expression of FXI gene, wherein polynucleic acid molecule comprises an antisense strand comprising the nucleotide sequence of asUfsaaauGfucuuUfgUfuGfcaagcsgsc (SEQ ID NO: 206) and a sense strand comprising the nucleotide sequence of gscsuugcAfaCfaAfagacauuuau (SEQ ID NO: 161), wherein “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “of” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “invdT” refers to 3′ inverted thymidine, “i” refers to 2′-O-methylinosine-3′-phosphate, “a4” refers to 2′-O-methyl-8-bromo-adenosine-3′-phopshate.

Further provided herein is a polynucleic acid molecule for modulating expression of FXI gene, wherein polynucleic acid molecule comprises an antisense strand comprising the nucleotide sequence of usUfsauagUfuuauGfcCfcUfucaugsusc (SEQ ID NO: 207) and a sense strand comprising the nucleotide sequence of csasugaaGfgGfcAfuaaacuauaa (SEQ ID NO: 163), wherein “A” refers to adenosine-3′-phosphate; “a” refers to 2′-O-methyladenosine-3′-phosphate, “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate, “s” refers to 3′-phosphorothioate, “invdT” refers to 3′ inverted thymidine, “i” refers to 2′-O-methylinosine-3′-phosphate, “a4” refers to 2′-O-methyl-8-bromo-adenosine-3′-phopshate.

Further provided herein is a polynucleic acid molecule for modulating expression of FXI gene, wherein polynucleic acid molecule comprises an antisense strand comprising the nucleotide sequence of asUfsagguAfaaaaAfcUfgGfcagcgsgsa (SEQ ID NO: 209) and a sense strand comprising the nucleotide sequence of csgscugcCfaGfuUfuuuuaccuau (SEQ ID NO: 185), wherein “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “of” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “invdT” refers to 3′ inverted thymidine, “i” refers to 2′-O-methylinosine-3′-phosphate, “a4” refers to 2′-O-methyl-8-bromo-adenosine-3′-phopshate.

Further provided herein is a polynucleic acid molecule for modulating expression of FXI gene, wherein polynucleic acid molecule comprises an antisense strand comprising the nucleotide sequence of isUfsaaaugucuuUfgUfuGfcaagcsgsc (SEQ ID NO: 217) and a sense strand comprising the nucleotide sequence of gscsuugcAfaCfaAfagacauuuau (SEQ ID NO: 161), wherein “A” refers to adenosine-3′-phosphate; “a” refers to 2′-O-methyladenosine-3′-phosphate, “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-m ethylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate, “s” refers to 3′-phosphorothioate, “invdT” refers to 3′ inverted thymidine, “i” refers to 2′-O-methylinosine-3′-phosphate, “a4” refers to 2′-O-methyl-8-bromo-adenosine-3′-phopshate.

Further provided herein is a polynucleic acid molecule for modulating expression of FXI gene, wherein polynucleic acid molecule comprises an antisense strand comprising the nucleotide sequence of usUfsaaauGfucuuUfgUfuGfcaagcsgsc (SEQ ID NO: 218) and a sense strand comprising the nucleotide sequence of gscsuugcAfaCfaAfagacauuuaa (SEQ ID NO: 222), wherein “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “of” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “invdT” refers to 3′ inverted thymidine, “i” refers to 2′-O-methylinosine-3′-phosphate, “a4” refers to 2′-O-methyl-8-bromo-adenosine-3′-phopshate.

Further provided herein is a polynucleic acid molecule for modulating expression of FXI gene, wherein polynucleic acid molecule comprises an antisense strand comprising the nucleotide sequence of a4sUfsaaauGfucuuUfgUfuGfcaagcsgsc (SEQ ID NO: 219) and a sense strand comprising the nucleotide sequence of gscsuugcAfaCfaAfagacauuuau (SEQ ID NO: 161), wherein “A” refers to adenosine-3′-phosphate; “a” refers to 2′-O-methyladenosine-3′-phosphate, “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate, “s” refers to 3′-phosphorothioate, “invdT” refers to 3′ inverted thymidine, “i” refers to 2′-O-methylinosine-3′-phosphate, “a4” refers to 2′-O-methyl-8-bromo-adenosine-3′-phopshate.

### Conjugation

### Targeting Moiety

In certain aspects, the polynucleotide molecule described herein is coupled or conjugated with one or more targeting moieties to form a polynucleotide-targeting moiety conjugate molecule. In some instances, a targeting moiety is selected based on its ability to target the conjugate molecule described herein to a desired cell population, tissue, or an organ selectively or preferably. In some instances, the targeting moiety targets the cell, tissue, or an organ that expresses the corresponding binding partner (e.g., either the corresponding receptor or ligand) of the targeting moiety. For example, the polynucleotide molecule conjugated with N-acetyl galactosamine (GalNAc) can target hepatocytes expressing asialoglycoprotein (ASGP-R). Any suitable GalNAc molecules that are known in the art to be used as a targeting moiety are contemplated. Exemplary GalNAc molecule includes a triantennary GalNAc (e.g., L96). A further example of the targeting moiety is galactose. The targeting moiety can also be a lipid, peptide, or small molecule.

A targeting moiety (i.e., an intracellular targeting moiety) that targets a desired site within the cell (e.g., endoplasmic reticulum, Golgi apparatus, nucleus, or mitochondria) may be included in the hybridized polynucleotide constructs disclosed herein. Non-limiting examples of the intracellular targeting moieties are provided in WO 2015/069932 and in WO 2015/188197; the disclosure of the intracellular targeting moieties in WO 2015/069932 and in WO 2015/188197 is incorporated herein by reference.

The polynucleotide molecule described herein, thus, may include one or more targeting moieties selected from the group consisting of intracellular targeting moieties, extracellular targeting moieties, and combinations thereof. Thus, the inclusion of one or more targeting moieties (e.g., extracellular targeting moieties including targeting moieties independently selected from the group consisting of folate, mannose, N-acetyl galactosamine, and prostate specific membrane antigen) and one or more intracellular targeting moiety (e.g., a moiety targeting endoplasmic reticulum, Golgi apparatus, nucleus, or mitochondria) in the polynucleotide molecule described herein can facilitate the delivery of the polynucleotides to a specific site within the specific cell population. In some aspects, the targeting moiety contains one or more mannose carbohydrates. Mannose targets the mannose receptor, which is a 175 KDa membrane-associated receptor that is expressed on sinusoidal liver cells and antigen presenting cells (e.g., macrophages and dendritic cells). It is a highly effective endocytotic/recycling receptor that binds and internalizes mannosylated pathogens and proteins (Lennartz et. al. J. Biol. Chem. 262:9942-9944, 1987; Taylor et. al. J. Biol. Chem. 265:12156-62, 1990).

Some of the targeting moieties are described herein. In some aspects, the targeting moiety contains or specifically binds to a protein selected from the group including insulin, insulin-like growth factor receptor 1 (IGF1R), IGF2R, insulin-like growth factor (IGF; e.g., IGF 1 or 2), mesenchymal epithelial transition factor receptor (c-met; also known as hepatocyte growth factor receptor (HGFR)), hepatocyte growth factor (HGF), epidermal growth factor receptor (EGFR), epidermal growth factor (EGF), heregulin, fibroblast growth factor receptor (FGFR), platelet-derived growth factor receptor (PDGFR), platelet-derived growth factor (PDGF), vascular endothelial growth factor receptor (VEGFR), vascular endothelial growth factor (VEGF), tumor necrosis factor receptor (TNFR), tumor necrosis factor alpha (TNF-α), TNF-β, folate receptor (FOLK), folate, transferrin, transferrin receptor (TfR), mesothelin, Fc receptor, c-kit receptor, c-kit, an integrin (e.g., an α4 integrin or a β-1 integrin), P-selectin, sphingosine-1-phosphate receptor-1 (S1PR), hyaluronate receptor, leukocyte function antigen-1 (LFA-1), CD4, CD11, CD18, CD20, CD25, CD27, CD52, CD70, CD80, CD85, CD95 (Fas receptor), CD106 (vascular cell adhesion molecule 1 (VCAM1), CD166 (activated leukocyte cell adhesion molecule (ALCAM)), CD178 (Fas ligand), CD253 (TNF-related apoptosis-inducing ligand (TRAIL)), ICOS ligand, CCR2, CXCR3, CCR5, CXCL12 (stromal cell-derived factor 1 (SDF-1)), interleukin 1 (IL-1), IL-1ra, IL-2, IL-3, IL-4, IL-6, IL-7, IL-8, CTLA-4, MART-1, gp100, MAGE-1, ephrin (Eph) receptor, mucosal addressin cell adhesion molecule 1 (MAdCAM-1), carcinoembryonic antigen (CEA), LewisY, MUC-1, epithelial cell adhesion molecule (EpCAM), cancer antigen 125 (CA125), prostate specific membrane antigen (PSMA), TAG-72 antigen, and fragments thereof. In further aspects, the targeting moiety contains erythroblastic leukemia viral oncogene homolog (ErbB) receptor (e.g., ErbB1 receptor; ErbB2 receptor; ErbB3 receptor; and ErbB4 receptor). In some aspects, the targeting moiety contains one or more (e.g., from 1 to 6) N-acetyl galactosamines (GalNAc). In certain aspects, the targeting moiety contains one or more (e.g., from 1 to 6) galactose. In certain aspects, the targeting moiety contains one or more (e.g., from 1 to 6) mannoses. In other aspects, the contains a folate ligand. The folate has the structure:

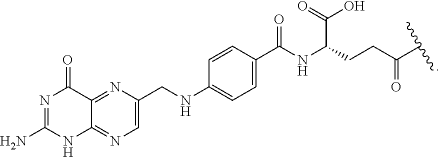 [SMILES: *C(=O)CC[C@H](NC(=O)c1ccc(NCc2cnc3[nH]c(N)nc(=O)c3n2)cc1)C(=O)O]

Certain targeting moieties may include bombesin, gastrin, gastrin-releasing peptide, tumor growth factors (TGF) (e.g., TGF-α or TGF-β), or vaccinia virus growth factor (VVGF). Non-peptidyl targeting moieties can also be used in the targeting moieties and may include, for example, steroids, carbohydrates, vitamins, and lectins. Some targeting moieties may include a polypeptide, such as somatostatin or somatostatin analog (e.g., octreotide or lanreotide), bombesin, or an antibody or antigen-binding fragment thereof. Antibodies may be of any recognized class or subclass, e.g., IgG, IgA, IgM, IgD, or IgE. Typical are those antibodies which fall within the IgG class. The antibodies can be derived from any species according techniques known in the art. Typically, however, the antibody is of human, murine, or rabbit origin. In addition, the antibody may be polyclonal or monoclonal, but is typically monoclonal. Human or chimeric (e.g., humanized) antibodies may be used in targeting moieties. Targeting moieties may include an antigen-binding fragment of an antibody. Such antibody fragments may include, for example, the Fab′, F(ab′)2, Fv, or Fab fragments, single domain antibody, ScFv, or other antigen-binding fragments. Fc fragments may also be employed in targeting moieties. Such antibody fragments can be prepared, for example, by proteolytic enzyme digestion, for example, by pepsin or papain digestion, reductive alkylation, or recombinant techniques. The materials and methods for preparing antibody fragments are well-known to those skilled in the art. See, e.g., Parham, J. Immunology, 131:2895, 1983; Lamoyi et al., J. Immunological Methods, 56:235, 1983.

Other peptides for use as a targeting auxiliary moiety in polynucleotide molecule described herein can be selected from KiSS peptides and analogs, urotensin II peptides and analogs, GnRH I and II peptides and analogs, depreotide, vapreotide, vasoactive intestinal peptide (VIP), cholecystokinin (CCK), RGD-containing peptides, melanocyte-stimulating hormone (MSH) peptide, neurotensin, calcitonin, glutathione, YIGSR (leukocyte-avid peptides, e.g., P483 H, which contains the heparin-binding region of platelet factor-4 (PF-4) and a lysine-rich sequence), atrial natriuretic peptide (ANP), β-amyloid peptides, delta-opioid antagonists (such as ITIPP(psi)), annexin-V, endothelin, leukotrieneB4 (LTB4), chemotactic peptides (e.g., N-formyl-methionyl-leucyl-phenylalanine-lysine (fMLFK), GP IIb/IIIa receptor antagonists (e.g., DMP444), human neutrophil elastase inhibitor (EPI-HNE-2 and EPI-HNE-4), plasmin inhibitor, antimicrobial peptides, apticide (P280 and P274), thrombospondin receptor (including analogs such as TP-1300), bitistatin, pituitary adenylyl cyclase type I receptor (PAC1), fibrin α-chain, peptides derived from phage display libraries, and conservative substitutions thereof.

One or more (e.g., from 1 to 6) targeting moieties can be linked to MOIETY or to X2 in formula (V′, V″, V′″, V″″, V″″′, V″″″) through -LinkA-.

In some aspects, the targeting moiety includes one or more (e.g., from 1 to 6 or from 1 to 3) asialoglycoprotein receptor ligands (e.g., GalNAc). In some aspects, an asialoglycoprotein receptor ligand (e.g., GalNAc) is attached to -LinkA- through an anomeric carbon (e.g., where the anomeric carbon is the carbon atom in an acetal or a hemiaminal). In some aspects, an asialoglycoprotein receptor ligand (e.g., GalNAc) comprises an anomeric carbon bonded to trivalent, tetravalent linker, pentavalent, or hexavalent linker, wherein the anomeric carbon is part of a hemiaminal group. An asialoglycoprotein receptor ligand (e.g., GalNAc) attached to a linker through a hemiaminal may produce a hybridized polynucleotide construct having superior efficacy in gene silencing as compared to hybridized polynucleotide constructs having the asialoglycoprotein receptor ligand (e.g., GalNAc) attached to a linker through an acetal.

In some aspects, the linker and three asialoglycoprotein receptor targeting moieties, each of which comprises GalNAc, are as shown in Formula (V). In some instances, the conjugate described herein only comprises one asialoglycoprotein receptor targeting moiety, so the conjugate comprises a structure of Formula (V) with any two of the targeting moieties removed. In some instances, the conjugate described herein only comprises two asialoglycoprotein receptor targeting moieties, so the conjugate described herein comprises a structure of Formula (V) with any one of the targeting moieties removed.

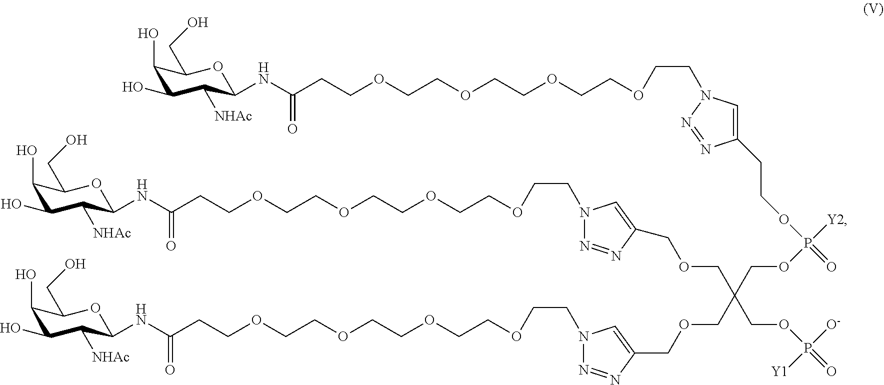

wherein one of Y1 and Y2 is nucleotide, or wherein both Y1 and Y2 are nucleotides and Y1 and Y2 are consecutive or neighboring nucleotides from the polynucleic acid molecule described herein.

In some aspects, the linker and the targeting moieties described herein are conjugated to 3′ end of the sense strand (e.g., as shown in Formula (V′, V″, V″″′, V′″″)). In some aspects, the linker and the targeting moieties described herein are conjugated to 5′ end of the sense strand (e.g., as shown in Formula (V″) or (V″′)). In some aspects, the linker and the targeting moieties described herein are conjugated to 3′ end of the antisense strand (e.g., as shown in Formula (V′), (V″″), (V″″′), (V″″″)). In some aspects, the linker and the targeting moieties described herein are conjugated to 5′ end of the antisense strand (e.g., as shown in Formula (V″) or (V″′)).

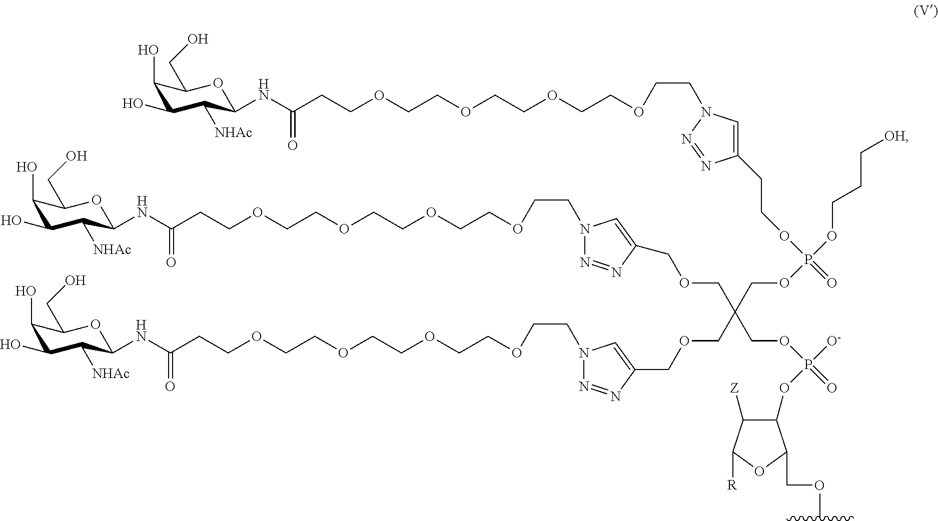

wherein Z in formula (V′) corresponds to one of the sugar modifications described herein (e.g., —H, —OH, —O-Methyl, —F, or —O-methoxyethyl), and R in formula (V′) is adenine, uracil, guanine, cytosine, thymine, abasic, or others.

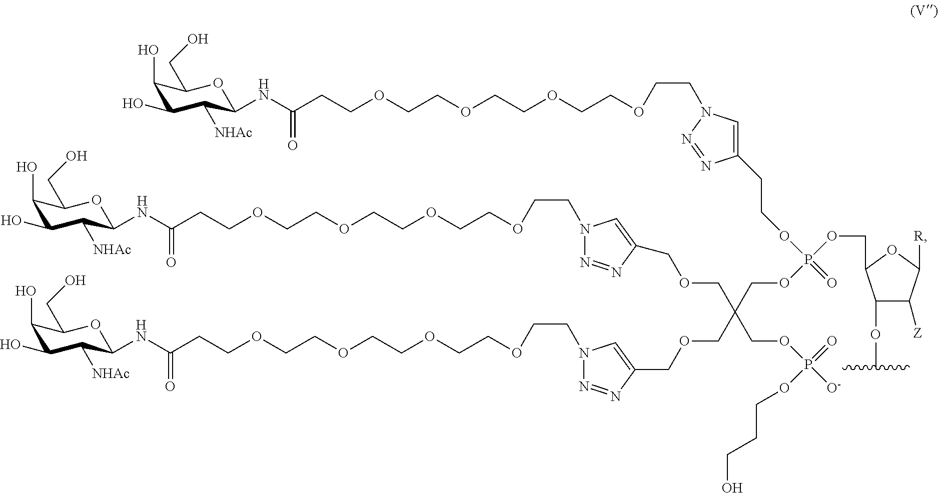

wherein Z in formula (V″) is a moiety that corresponds to one of the sugar modifications described herein (e.g., —H, —OH, —O-Methyl, —F, or —O-methoxyethyl) and R in formula (V″) is adenine, uracil, guanine, cytosine, thymine, abasic, or others.

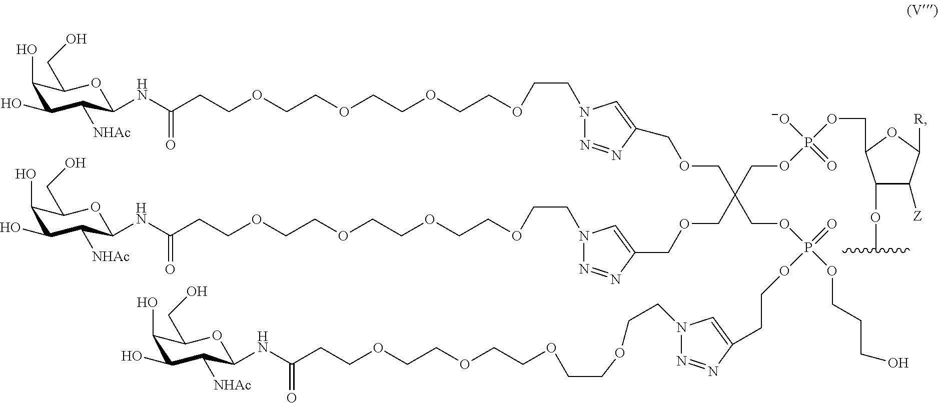

wherein Z in formula (V″′) is a moiety that corresponds to one of the sugar modifications described herein (e.g., —H, —OH, —O-Methyl, —F, or —O-methoxyethyl) and R in formula (V″′) is adenine, uracil, guanine, cytosine, thymine, abasic, or others.

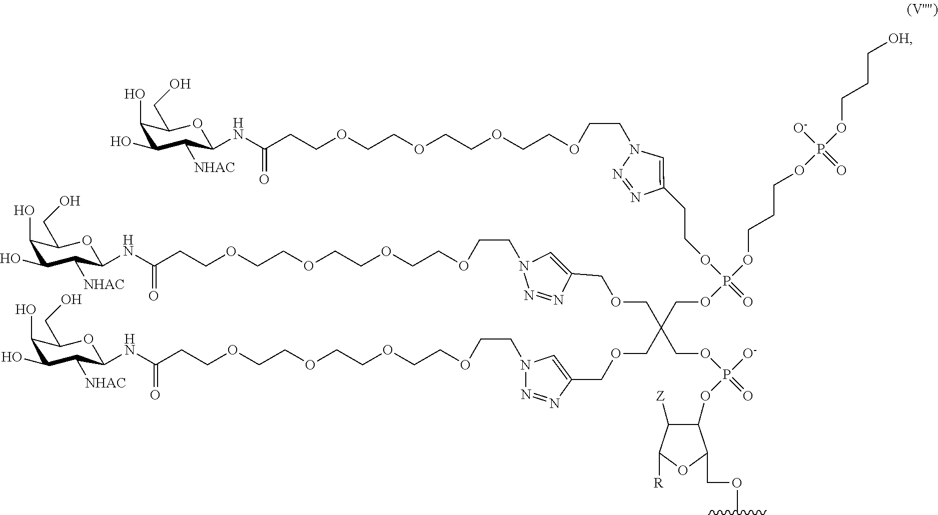

wherein Z in formula (V″″) is a moiety that corresponds to one of the sugar modifications described herein (e.g., —H, —OH, —O-Methyl, —F, or —O-methoxyethyl) and R in formula (V″″) is or others.

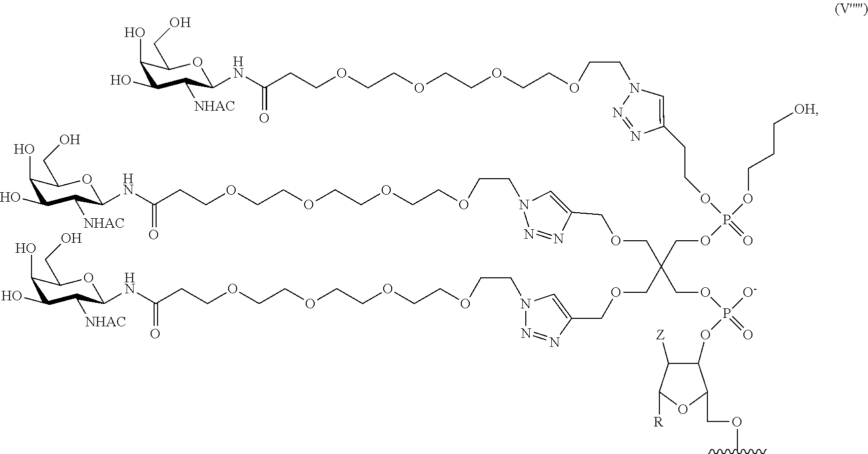

wherein Z in formula (V″″′) is a moiety that corresponds to one of the sugar modifications described herein (e.g., —H, —OH, —O-Methyl, —F, or —O-methoxyethyl) and R in formula (V″″′) is adenine, uracil, guanine, cytosine, thymine, abasic, or others.

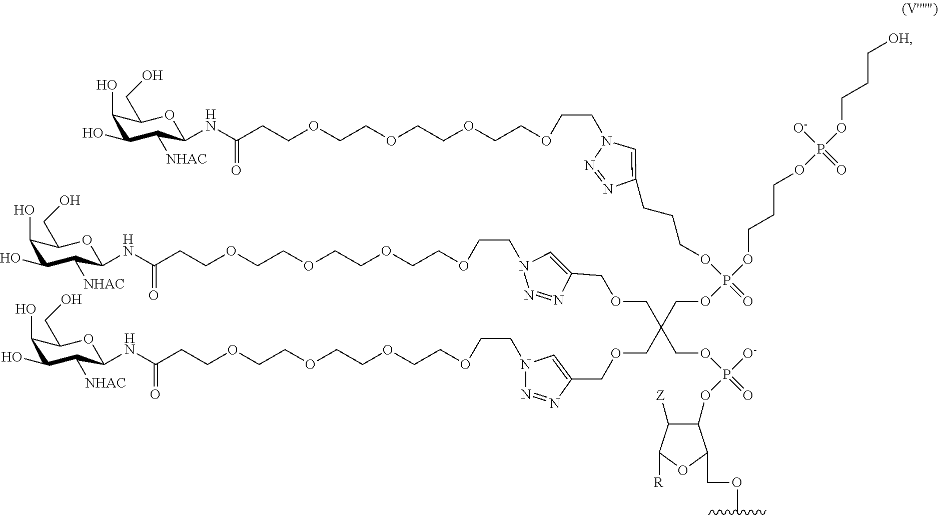

wherein Z in formula (V″″″) is a moiety that corresponds to one of the sugar modifications described herein (e.g., —H, —OH, —O-Methyl, —F, or —O-methoxyethyl) and R in formula (V″″″) is adenine, uracil, guanine, cytosine, thymine, abasic, or others.

In some instances, the 3′ end of passenger/sense strand of a polynucleic acid molecule from Tables 1-2 and Tables 7-9 is conjugated with X2-GalNAc (see Formula (V), (V′), (V″″), (V″″′), (V″″″)). In some instances, the 5′ end of passenger/sense strand of a polynucleic acid molecule from Tables 1-2 and Tables 7-9 is conjugated with X2-GalNAc (see Formula (V), (V″), or (V″′)). In some instances, a nucleic acid within passenger/sense strand (not at the 5′ or 3′ end) from Table 1 or Table 2 is conjugated with X2-GalNAc (see Formula (V)). In some instances, the 3′ end of guide/antisense strand of a polynucleic acid molecule from Tables 1-2 and Tables 7-9 is conjugated with X2-GalNAc (see Formula (V), (V′), (V″′), (V″″), (V″″′)). In some instances, the 5′ end of guide/antisense strand of a polynucleic acid molecule from Tables 1-2 and Tables 7-9 is conjugated with X2-GalNAc (see Formula (V), (V″), or (V″′)). In some instances, a nucleic acid within guide/antisense strand (not at the 5′ or 3′ end) of a polynucleic acid molecule from Tables 1-2 and Tables 7-9 is conjugated with X2-GalNAc (see Formula (V)).

In some instances, one or more endosomal escape moieties (e.g., from 1 to 6 or from 1 to 3) can be attached to a polynucleotide construct or a hybridized polynucleotide construct disclosed herein as an auxiliary moiety. Exemplary endosomal escape moieties include chemotherapeutics (e.g., quinolones such as chloroquine); fusogenic lipids (e.g., dioleoylphosphatidyl-ethanolamine (DOPE)); and polymers such as polyethylenimine (PEI); poly(beta-aminoester)s; polypeptides, such as polyarginines (e.g., octaarginine) and polylysines (e.g., octalysine); proton sponges, viral capsids, and peptide transduction domains as described herein. For example, fusogenic peptides can be derived from the M2 protein of influenza A viruses; peptide analogs of the influenza virus hemagglutinin; the HEF protein of the influenza C virus; the transmembrane glycoprotein of filoviruses; the transmembrane glycoprotein of the rabies virus; the transmembrane glycoprotein (G) of the vesicular stomatitis virus; the fusion protein of the Sendai virus; the transmembrane glycoprotein of the Semliki forest virus; the fusion protein of the human respiratory syncytial virus (RSV); the fusion protein of the measles virus; the fusion protein of the Newcastle disease virus; the fusion protein of the visna virus; the fusion protein of murine leukemia virus; the fusion protein of the HTL virus; and the fusion protein of the simian immunodeficiency virus (SIV). Other moieties that can be employed to facilitate endosomal escape are described in Dominska et al., Journal of Cell Science, 123(8):1183-1189, 2010. Specific examples of endosomal escape moieties including moieties suitable for conjugation to the hybridized polynucleotide constructs disclosed herein are provided, e.g., in WO 2015/188197; the disclosure of these endosomal escape moieties is incorporated by reference herein.

One or more endosomal escape moieties (e.g., from 1 to 6 or from 1 to 3) can be attached to a MOIETY or X2 in formula (V′, V″, V″′, V″″, V″″′, or V″″″) through -LinkA-, as described herein.

One or more cell penetrating peptides (CPP) (e.g., from 1 to 6 or from 1 to 3) can be attached to a polynucleotide construct or a hybridized polynucleotide construct disclosed herein as an auxiliary moiety. The CPP can be linked to the hybridized polynucleotide bioreversibly through a disulfide linkage, as disclosed herein. Thus, upon delivery to a cell, the CPP can be cleaved intracellularly, e.g., by an intracellular enzyme (e.g., protein disulfide isomerase, thioredoxin, or a thioesterase) and thereby release the polynucleotide.

CPPs are known in the art (e.g., TAT or Arg8) (Snyder and Dowdy, 2005, Expert Opin. Drug Deliv. 2, 43-51). Specific examples of CPPs including moieties suitable for conjugation to the hybridized polynucleotide constructs disclosed herein are provided, e.g., in WO 2015/188197; the disclosure of these CPPs is incorporated by reference herein.

CPPs are positively charged peptides that are capable of facilitating the delivery of biological cargo to a cell. It is believed that the cationic charge of the CPPs is essential for their function. Moreover, the transduction of these proteins does not appear to be affected by cell type, and these proteins can efficiently transduce nearly all cells in culture with no apparent toxicity (Nagahara et al., Nat. Med. 4:1449-52, 1998). In addition to full-length proteins, CPPs have also been used successfully to induce the intracellular uptake of DNA (Abu-Amer, supra), antisense polynucleotides (Astriab-Fisher et al., Pharm. Res, 19:744-54, 2002), small molecules (Polyakov et al., Bioconjug. Chem. 11:762-71, 2000) and even inorganic 40 nm iron particles (Dodd et al., J. Immunol. Methods 256:89-105, 2001; Wunderbaldinger et al., Bioconjug. Chem. 13:264-8, 2002; Lewin et al., Nat. Biotechnol. 18:410-4, 2000; Josephson et al., Bioconjug. Chem. 10:186-91, 1999) suggesting that there is considerable flexibility in particle size in this process.

In one cases, a CPP useful in the methods and compositions as described herein includes a peptide featuring substantial alpha-helicity. It has been discovered that transfection is optimized when the CPP exhibits significant alpha-helicity. In another case, the CPP includes a sequence containing basic amino acid residues that are substantially aligned along at least one face of the peptide. A CPP described herein may be a naturally occurring peptide or a synthetic peptide.

One or more cell penetrating peptides (e.g., from 1 to 6 or from 1 to 3) can be attached to a MOIETY or X2 in formula (V′, V″, V″′, V″″, V″″′, V″″″) through -LinkA-, as described herein.

The polynucleotide constructs and the hybridized polynucleotide constructs disclosed herein can also include covalently attached neutral polymer-based auxiliary moieties. Neutral polymers include poly(C1-6 alkylene oxide), e.g., poly(ethylene glycol) and poly(propylene glycol) and copolymers thereof, e.g., di- and triblock copolymers. Other examples of polymers include esterified poly(acrylic acid), esterified poly(glutamic acid), esterified poly(aspartic acid), poly(vinyl alcohol), poly(ethylene-co-vinyl alcohol), poly(N-vinyl pyrrolidone), poly(ethyloxazoline), poly(alkylacrylates), poly(acrylamide), poly(N-alkylacrylamides), poly(N-acryloylmorpholine), poly(lactic acid), poly(glycolic acid), poly(dioxanone), poly(caprolactone), styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyurethane, N-isopropylacrylamide polymers, and poly(N,N-dialkylacrylamides). Exemplary polymer auxiliary moieties may have molecular weights of less than 100, 300, 500, 1000, or 5000 Da (e.g., greater than 100 Da). Other polymers are known in the art.

One or more polymers (e.g., from 1 to 6 or from 1 to 3) can be attached to a MOIETY or X2 in formula (V′, V″, V″′, V″″, V″″′, V″″″) through -LinkA-, as described herein.

### Conjugation Linkers

In some aspects, the polynucleic acid molecules described herein comprises a sense or antisense strand bonded to at least one group of formula (I)

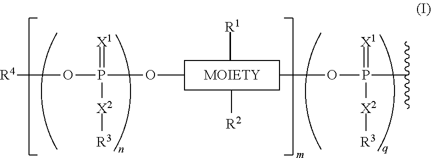

or a salt thereof, or a stereoisomer thereof,
where
each X¹ is independently O or S;
each X² is independently O, S, NH, or a bond;
MOIETY is optionally substituted C₂₋₁₀ alkane-tetrayl or a group -M¹-M²-M³-, wherein each M¹ and each M³ is independently absent or optionally substituted C₁₋₆ alkylene, and M² is optionally substituted C₃₋₉ heterocycle-tetrayl, optionally substituted C₆₋₁₀ arene-tetrayl, or optionally substituted C₃₋₈ cycloalkane-tetrayl;
each R¹ and each R² is independently H, optionally substituted C₁₋₁₆ alkyl, optionally substituted C₂₋₁₆ heteroalkyl, a conjugation moiety, or -LinkA(-T)ₚ, provided that at least one R¹ or at least one R² is a conjugation moiety or -LinkA(-T)ₚ;
each R³ is independently H, optionally substituted C₁₋₁₆ alkyl, optionally substituted C₂₋₁₆ heteroalkyl, optionally substituted C₂₋₁₆ alkenyl, optionally substituted C₂₋₁₆ alkynyl, optionally substituted (C₁₋₉ heterocyclyl)-C₁₋₆-alkyl, optionally substituted (C₆₋₁₀ aryl)-C₁₋₆-alkyl, optionally substituted (C₃₋₈ cycloalkyl)-C₁₋₆-alkyl, a conjugation moiety, or -LinkA(-T)ₚ;
R⁴ is H, optionally substituted C₁₋₆ alkyl, -LinkA(-T)ₚ, or -Sol;
each LinkA is independently a multivalent linker (e.g., including —C(O)—N(H)— (e.g., at least one multivalent linker including —C(O)—N(H)— bonded to T));
each T is independently an auxiliary moiety;
Sol is solid support;
m is an integer from 1 to 6;
each n is independently 0 or 1;
each p is independently an integer from 1 to 6, and
q is an integer from 0 to 3.

The at least one group of formula (I) may be bonded to a 5′-terminus, 3′-terminus, internucleoside phosphate, internucleoside phosphorothioate, or internucleoside phosphorodithioate of the polynucleotide. When the at least one group of formula (I) is bonded to the internucleoside phosphate, internucleoside phosphorothioate, or internucleoside phosphorodithioate, q is 0. The polynucleotide construct contains no more than one Sol.

Group -LinkA- can include from 0 to 3 multivalent monomers (e.g., optionally substituted C1-6 alkane-triyl, optionally substituted C1-6 alkane-tetrayl, or trivalent nitrogen atom) and one or more divalent monomers (e.g., from 1 to 40), where each divalent monomer is independently optionally substituted C1-6 alkylene; optionally substituted C2-6 alkenylene; optionally substituted C2-6 alkynylene; optionally substituted C3-8 cycloalkylene; optionally substituted C3-8 cycloalkenylene; optionally substituted C6-14 arylene; optionally substituted C1-9 heteroarylene having 1 to 4 heteroatoms selected from N, O, and S; optionally substituted C1-9 heterocyclylene having 1 to 4 heteroatoms selected from N, O, and S; imino; optionally substituted N; O; or S(O)m, wherein m is 0, 1, or 2. In some aspects, each monomer is independently optionally substituted C1-6 alkylene; optionally substituted C3-8 cycloalkylene; optionally substituted C3-8 cycloalkenylene; optionally substituted C6-14 arylene; optionally substituted C1-9 heteroarylene having 1 to 4 heteroatoms selected from N, O, and S; optionally substituted C1-9 heterocyclylene having 1 to 4 heteroatoms selected from N, O, and S; imino; optionally substituted N; O; or S(O)m, where m is 0, 1, or 2 (e.g., m is 2). In certain aspects, each monomer is independently optionally substituted C1-6 alkylene; optionally substituted C3 8 cycloalkylene; optionally substituted C3-8 cycloalkenylene; optionally substituted C6-14 arylene; optionally substituted C1-9 heteroarylene having 1 to 4 heteroatoms selected from N, O, and S; optionally substituted C1-9 heterocyclylene having 1 to 4 heteroatoms selected from N, O, and S; optionally substituted N; O; or S(O)m, where m is 0, 1, or 2 (e.g., m is 2). The non-bioreversible linker connecting the auxiliary moiety to the conjugating moiety or to the reaction product thereof can include from 2 to 500 (e.g., from 2 to 300 or from 2 to 200) of such monomers. Group -LinkA- may include a poly(alkylene oxide) (e.g., polyethylene oxide, polypropylene oxide, poly(trimethylene oxide), polybutylene oxide, poly(tetramethylene oxide), and diblock or triblock co-polymers thereof). In some aspects, the non-bioreversible linker includes polyethylene oxide (e.g., poly(ethylene oxide) having a molecular weight of less than 1 kDa).

Group -LinkA(-T)p in formula (I) may be prepared by a process described in the sections below. In some instances, -LinkA(-T)p is of formula (II):

-Q¹-Q²([-Q³-Q⁴-Q⁵]ₛQ⁶-T)ₚ, (II)
where
each s is independently an integer from 0 to 20 (e.g., from 0 to 10), where the repeating units are the same or different;
Q¹ is a conjugation linker (e.g., [-Q³-Q⁴-Q⁵]ₛ-Q^{C}-, where Q^{C} is optionally substituted C₂₋₁₂ heteroalkylene (e.g., a heteroalkylene containing —C(O)—N(H)—, —N(H)—C(O)—, —S(O)₂—N(H)—, or —N(H)—S(O)₂—), optionally substituted C₁₋₁₂ thioheterocyclylene (e.g.,

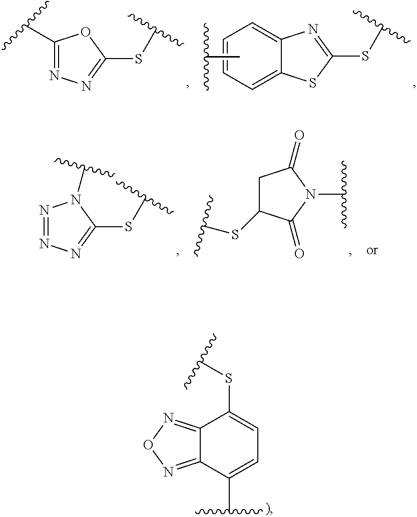

optionally substituted C₁₋₁₂ heterocyclylene (e.g., 1,2,3-triazole-1,4-diyl or

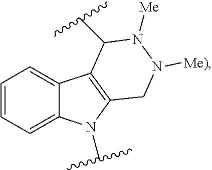

cyclobut-3-ene-1,2-dione-3,4-diyl, or pyrid-2-yl hydrazone);
Q² is a linear group (e.g., [-Q³-Q⁴-Q⁵]ₛ-), if p is 1, or a branched group (e.g., [-Q³-Q⁴-Q⁵]ₛ-Q⁷([-Q³-Q⁴-Q⁵]ₛ (Q⁷)ₚ₁)ₚ₂, where p1 is 0 or 1, p2 is 0, 1, 2, or 3), if p is an integer from 2 to 6;
each Q³ and each Q⁶ is independently absent, —CO—, —NH—, —O—, —S—, —SO₂—, —OC(O)—, —COO—, —NHC(O)—, —C(O)NH—, —CH₂—, —CH₂NH—, —NHCH₂—, —CH₂O—, or —OCH₂—;
each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₆₋₁₀ arylene, optionally substituted C₁₋₉ heteroarylene, or optionally substituted C₁₋₉ heterocyclylene;
each Q⁵ is independently absent, —CO—, —NH—, —O—, —S—, —SO₂—, —CH₂—, —C(O)O—, —OC(O)—, —C(O)NH—, —NH—C(O)—, —NH—CH(R^{a})—C(O)—, or —C(O)—CH(R^{a})—NH—;
each Q⁷ is independently optionally substituted C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl; and
each R^{a} is independently H or an amino acid side chain;
provided that at least one of Q³, Q⁴, and Q⁵ is present.

In some aspects, each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, or optionally substituted C₁₋₉ heterocyclylene. In certain aspects, s is 1, 2, 3, 4, 5, 6, 7, 8, 9, 10, 11, 12, 13, 14, 15, 16, 17, 18, 19, or 20.

Thus, in formula (II), LinkA may include a single branching point, if each p1 is 0, or multiple branching points, if at least one p1 is 1.

In formula (II), Q¹ may be —O-Q^{L}-Q^{C}-, where Q^{L} is optionally substituted C₂₋₁₂ heteroalkylene, optionally substituted C₁₋₁₂ alkylene, or -(optionally substituted C₁₋₆ alkylene)—(optionally substituted C₆₋₁₀ arylene)-. In some aspects, Q^{L} is optionally substituted C₂₋₁₂ heteroalkylene or optionally substituted C₁₋₁₂ alkylene. In formula (II), Q^{C} may be:

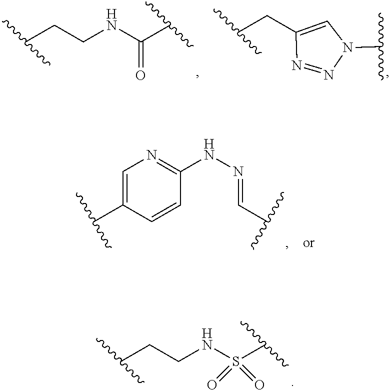 [SMILES: *CCNC(*)=O ; *Cc1cn(*)nn1 ; */C=N/Nc1ccc(*)cn1 ; *CCNS(*)(=O)=O]

In formula (II), Q² may be a linear group of formula [-Q³-Q⁴-Q⁵]ₛ-, where Q³, Q⁴, and Q⁵ are as defined for formula (II). Alternatively, Q² may be a branched group [-Q³-Q⁴-Q⁵]ₛ-Q⁷([-Q³-Q⁴-Q⁵]ₛ-(Q⁷)ₚ₁)ₚ₂, where each Q⁷ is independently optionally substituted C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl;
where
p1 is 0 or 1;
p2 is 0, 1, 2, or 3;
where,
   when p1 is 0, LinkA is a trivalent or tetravalent linker, and,
   when p1 is 1, LinkA is a tetravalent, pentavalent, or hexavalent linker.

In certain aspects, p1 is 0.

In some aspects, Q⁷ is:

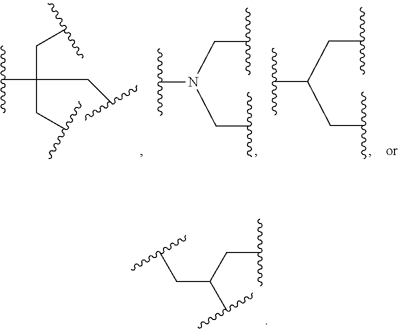 [SMILES: *CC(*)(C*)C* ; *CN(*)C* ; *CC(*)C* ; *CC(*)C*]

Compounds that may be used in the preparation of group -LinkA(-T)p in formula (I) are described herein as well as in WO 2015/188197. Non-limiting examples of -LinkA include:

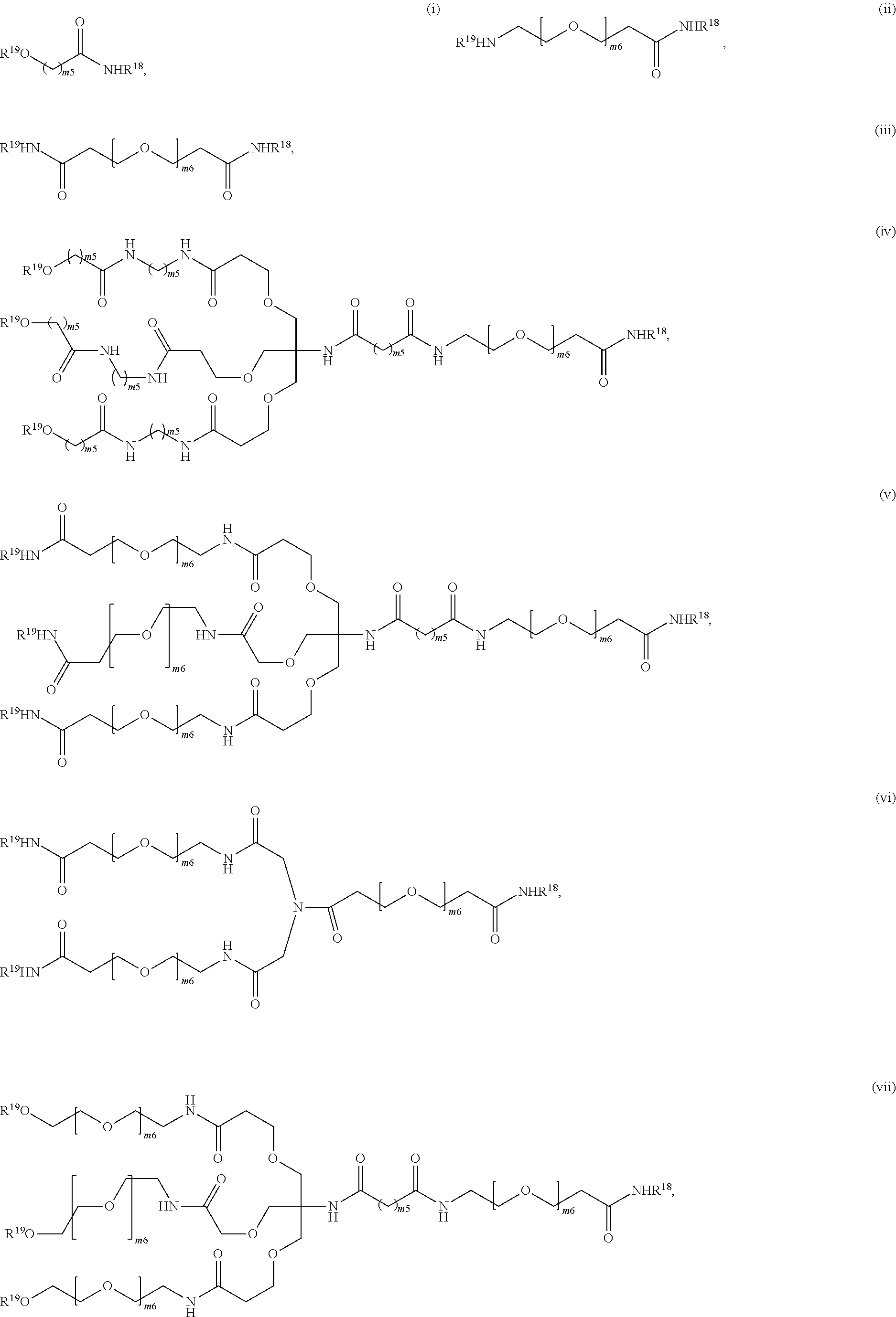

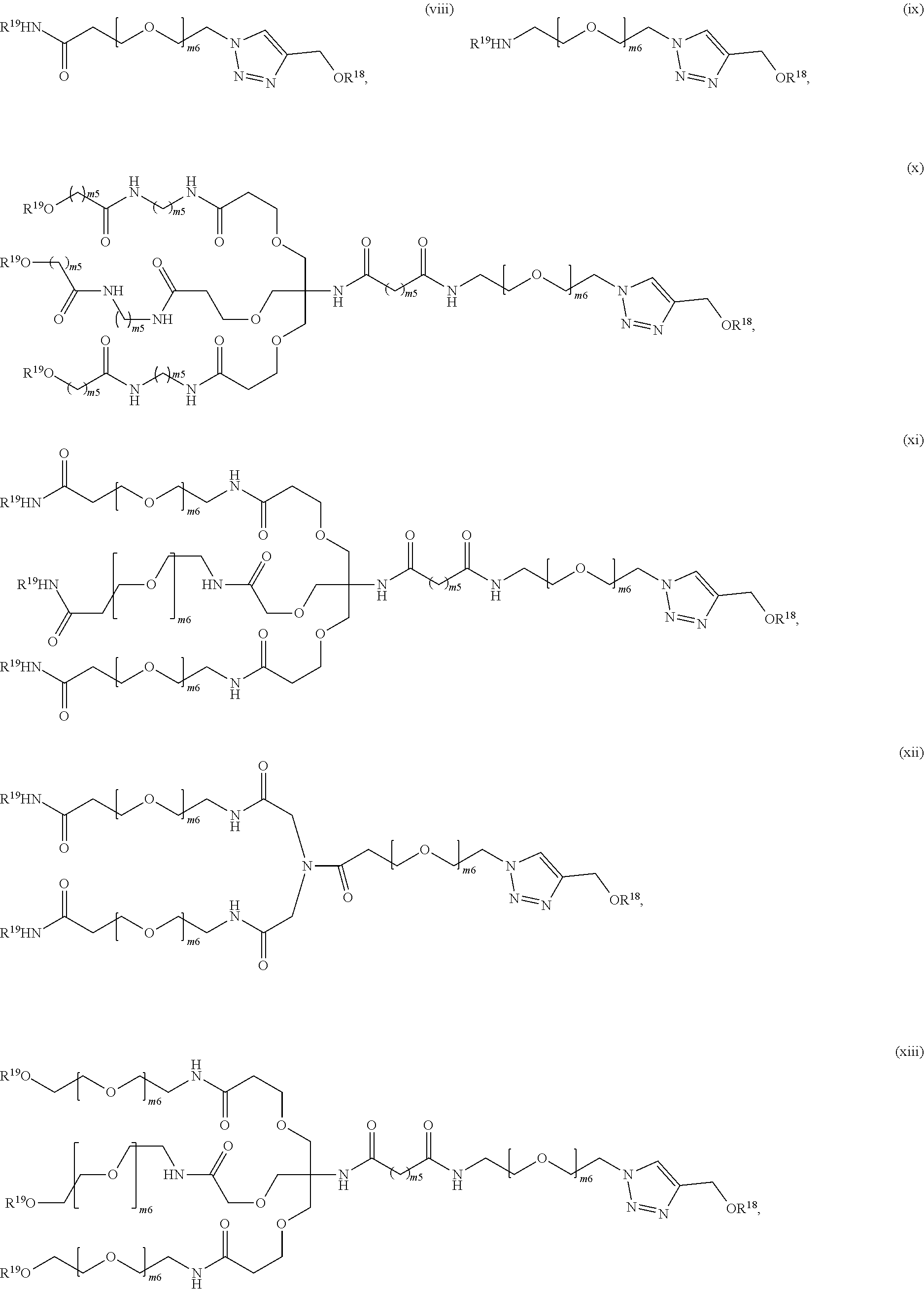

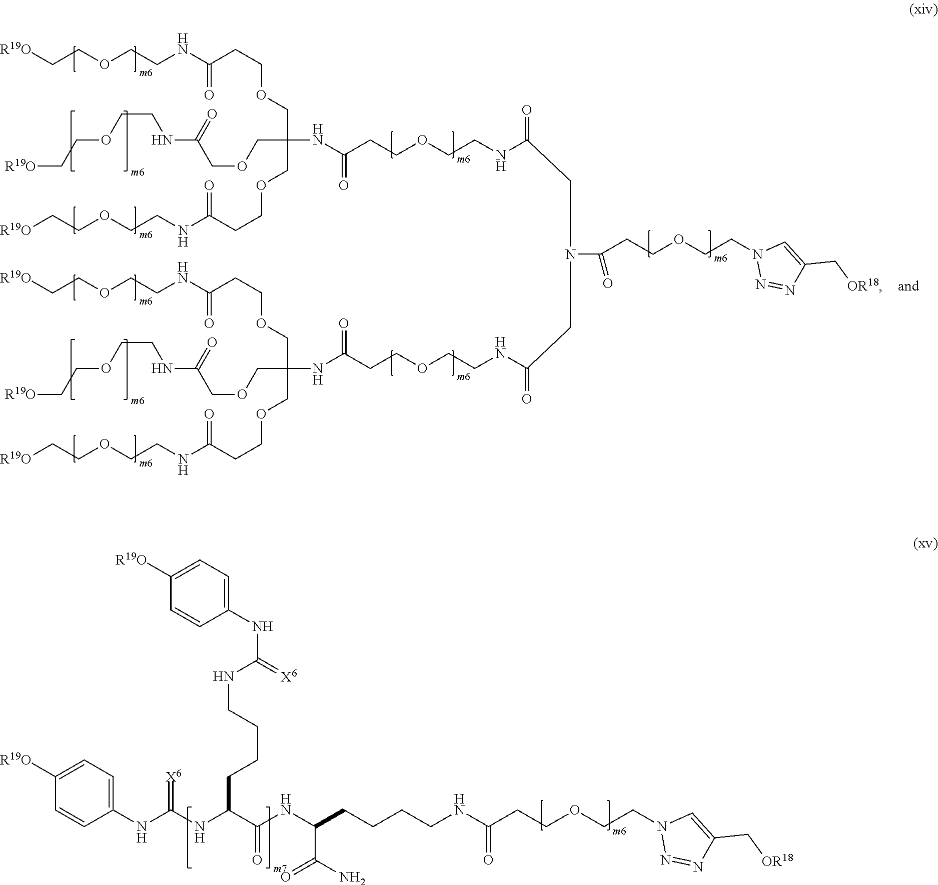

where
R¹⁸ is a bond to MOIETY,
each R¹⁹ is independently a bond to auxiliary moiety,
each m5 is independently an integer from 1 to 20,
each m6 is independently an integer from 1 to 10,
m7 is an integer from 1 to 6, and
each X⁶ is independently O or S.

In formula (II), when the conjugation linker is of formula [-Q³-Q⁴-Q⁵]ₛ-Q^{C}-, -Q²([-Q³-Q⁴-Q⁵]ₛ-Q⁶-T)ₚ may be:

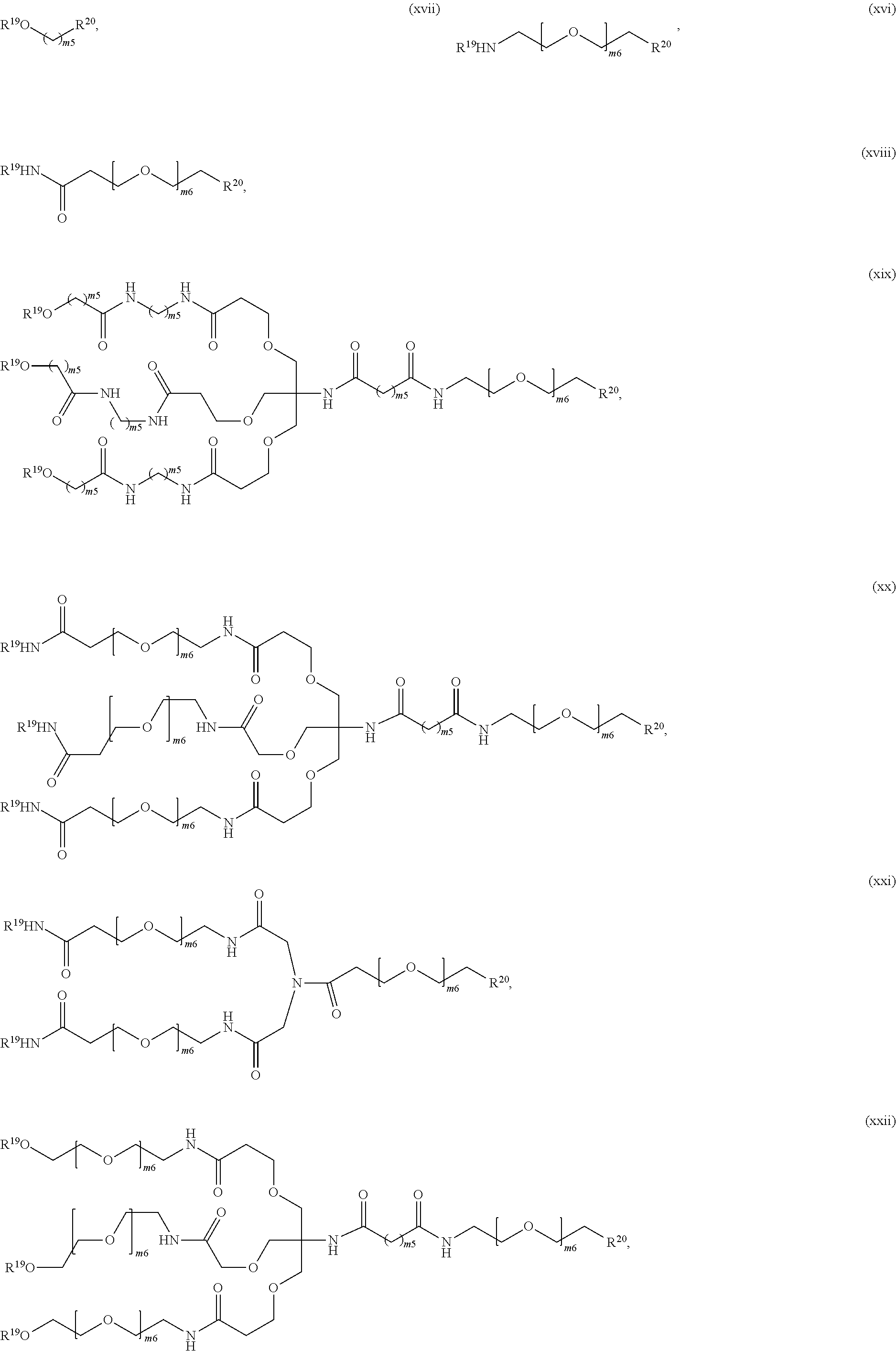

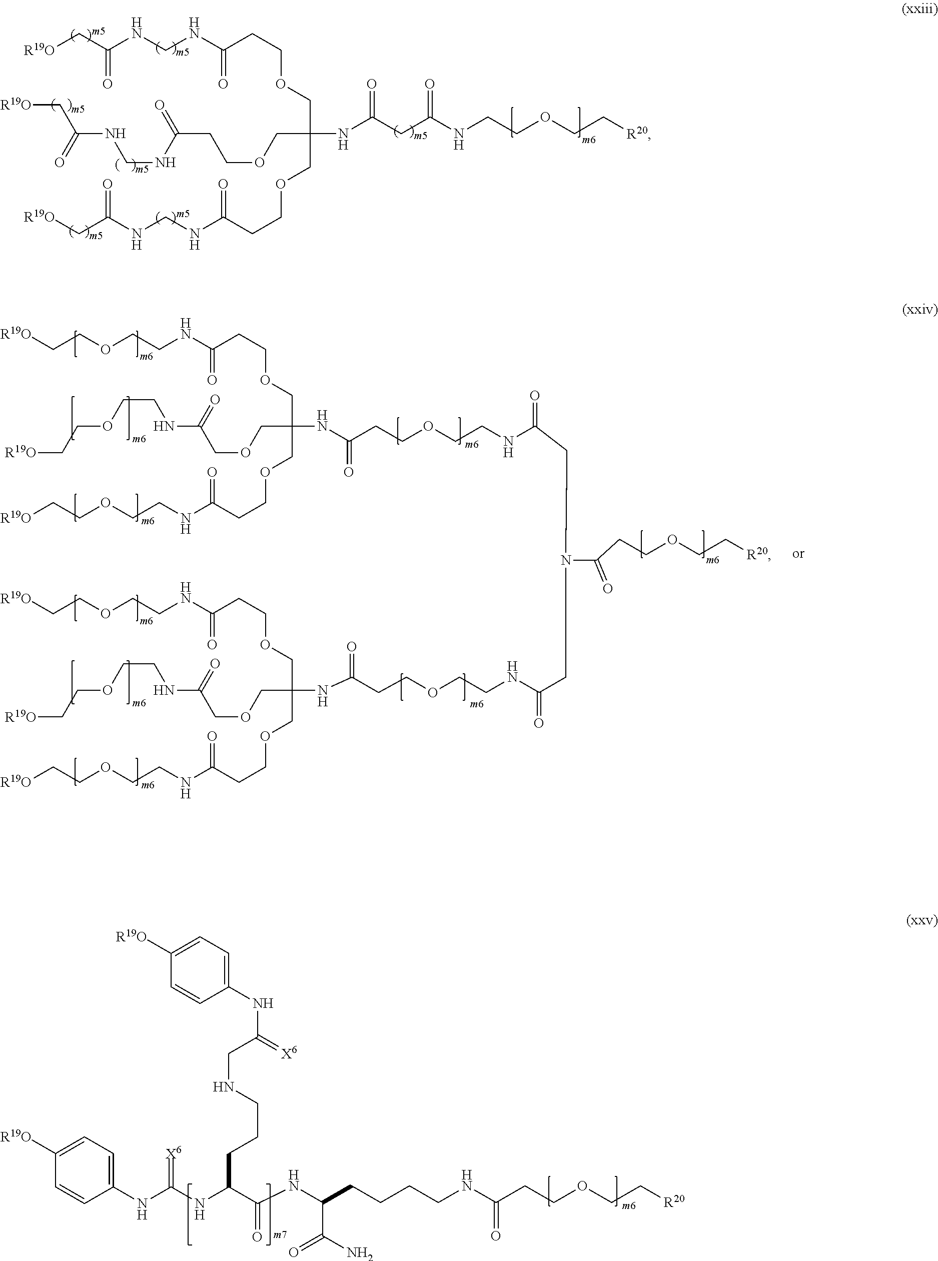

wherein
R²⁰ is a bond to Q^{C} in Q¹,
each R¹⁹ is independently a bond to an auxiliary moiety,
each m5 is independently an integer from 1 to 20,
each m6 is independently an integer from 1 to 10,
m7 is an integer from 1 to 6, and
each X⁶ is independently O or S.

In some aspects, the linker described herein is cleavable. In some aspects, the linker described herein is non-cleavable.

In some aspects, the polynucleic acid molecule described herein comprises a sense or antisense strand bonded to at least one group of formula (IV),

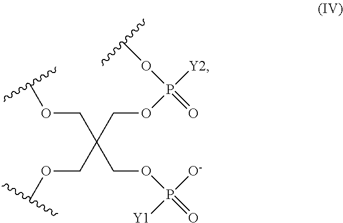

wherein at least one of Y1 or Y2 is a nucleotide from the polynucleic acid molecule.

In some instances, the Y1 is the last nucleotide on the 3′-terminus or the first nucleotide on the 5′-terminus of one of the strands of the polynucleic acid molecule. In some instances, the Y1 is the last nucleotide on the 3′-terminus or the first nucleotide on the 5′-terminus of the sense strand of the polynucleic acid molecule. In some instances, the Y1 is the last nucleotide on the 3′-terminus or the first nucleotide on the 5′-terminus of the sense strand of the polynucleic acid molecule, and the Y2 is a 3-hydroxy-propoxy group. In some instances, the Y2 is the first nucleotide on the 5′-terminus or the last nucleotide on the 3′-terminus of one of the strands of the polynucleic acid molecule. In some instances, the Y2 is the first nucleotide on the 5′ terminus or the last nucleotide on the 3′-terminus of the sense strand of the polynucleic acid molecule. In some instances, the Y2 is the first nucleotide on the 5′-terminus or the last nucleotide on the 3′-terminus of the sense strand of the polynucleic acid molecule, and the Y1 is a 3-hydroxy-propoxy group. In other instances, the Y1 and Y2 are two consecutive nucleotides in one of the strands of the polynucleic acid molecule.

In some aspects, the targeting moiety described herein is conjugated to 3′ end of the sense strand (e.g., formula (IV′) or (IV″″)). In some aspects, the targeting moiety described herein is conjugated to 5′ end of the sense strand (e.g., formula (IV″) or (IV″′)). In some aspects, the targeting moiety described herein is conjugated to 3′ end of the antisense strand (e.g., formula (IV′) or (IV″″)). In some aspects, the targeting moiety described herein is conjugated to 5′ end of the antisense strand (e.g., formula (IV″) or (IV″′)).

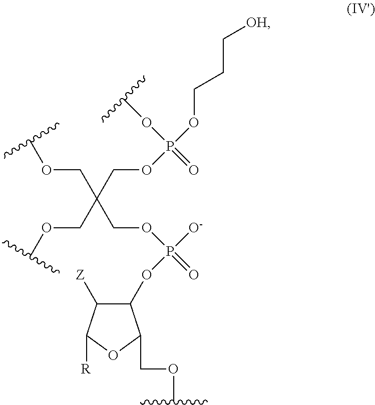

wherein Z in formula (IV′) is a moiety that corresponds to one of the sugar modifications described herein (e.g., —H, —OH, —O-Methyl, —F, or —O-methoxyethyl) and R in formula (IV′) is adenine, uracil, guanine, cytosine, thymine, abasic, or others.

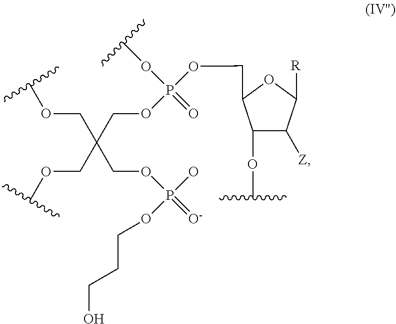

wherein Z in formula (IV″) is a moiety that corresponds to one of the sugar modifications described herein (e.g., —H, —OH, —O-Methyl, —F, or —O-methoxyethyl) and R in formula (IV″) is adenine, uracil, guanine, cytosine, thymine, abasic, or others.

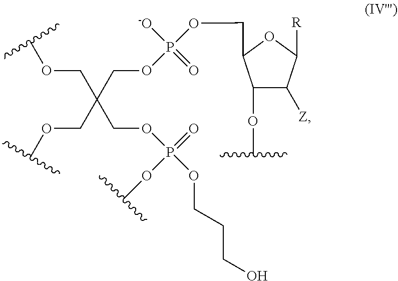

wherein Z in formula (IV″′) is a moiety that corresponds to one of the sugar modifications described herein (e.g., —H, —OH, —O-Methyl, —F, or —O-methoxyethyl) and R in formula (IV″′) is adenine, uracil, guanine, cytosine, thymine, abasic, or others.

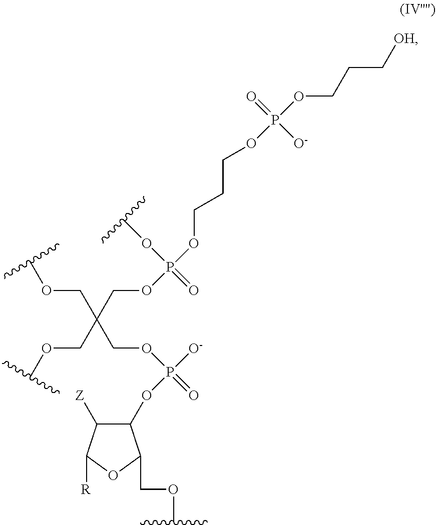

wherein Z in formula (IV″″) is a moiety that corresponds to one of the sugar modifications described herein (e.g., —H, —OH, —O-Methyl, —F, or —O-methoxyethyl) and R in formula (IV″″) is adenine, uracil, guanine, cytosine, thymine, abasic, or others.

### Pharmaceutical Compositions

Delivery of the polynucleotide molecules described herein can be achieved by contacting a cell with the polynucleotide molecules described herein using a variety of methods. In particular aspects, the polynucleotide molecule described herein is formulated with various excipients, vehicles, and carriers, as described more fully elsewhere herein.

A pharmaceutical composition described herein can be prepared to include a hybridized polynucleotide construct disclosed herein, into a form suitable for administration to a subject using carriers, excipients, and vehicles. Frequently used excipients include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti oxidants, chelating agents, and inert gases. Other pharmaceutically acceptable vehicles include aqueous solutions, non-toxic excipients, including salts, preservatives, buffers and the like, as described, for instance, in Remington: The Science and Practice of Pharmacy, 21st Ed., Gennaro, Ed., Lippencott Williams & Wilkins (2005), and The United States Pharmacopeia: The National Formulary (USP 36 NF31), published in 2013. The pH and exact concentration of the various components of the pharmaceutical composition are adjusted according to routine skills in the art. See Goodman and Gilman's, The Pharmacological Basis for Therapeutics.

The pharmaceutical compositions described herein may be administered locally or systemically. The therapeutically effective amounts will vary according to factors, such as the degree of infection in a subject, the age, sex, and weight of the individual. Dosage regimes can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g., subcutaneous, intravenous, intraorbital, and the like), oral administration, ophthalmic application, inhalation, topical application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically the composition will be stable under the conditions of manufacture and storage and preserved against the contaminating action of microorganisms, such as bacteria and fungi. The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

It is especially advantageous to formulate parenteral compositions in dosage unit form for ease of administration and uniformity of dosage. Dosage unit form as used herein, refers to physically discrete units suited as unitary dosages for the subject to be treated; each unit containing a predetermined quantity of pharmaceutical composition is calculated to produce the desired therapeutic effect in association with the required pharmaceutical vehicle. The specification for the dosage unit forms is related to the characteristics of the pharmaceutical composition and the particular therapeutic effect to be achieve. The principal pharmaceutical composition is compounded for convenient and effective administration in effective amounts with a suitable pharmaceutically acceptable vehicle in an acceptable dosage unit. In the case of compositions containing supplementary active ingredients, the dosages are determined by reference to the usual dose and manner of administration of the ingredients.

The pharmaceutical composition can be orally administered, for example, in a carrier, e.g., in an enteric-coated unit dosage form. The pharmaceutical composition and other ingredients can also be enclosed in a hard or soft-shell gelatin capsule or compressed into tablets. For oral therapeutic administration, the pharmaceutical composition can be incorporated with excipients and used in the form of ingestible tablets, troches, capsules, pills, wafers, and the like. Such compositions and preparations should contain at least 1% by weight of active compound. The percentage of the compositions and preparations can, of course, be varied and can conveniently be between about 5% to about 80% of the weight of the unit. The tablets, troches, pills, capsules, and the like can also contain the following: a binder, such as gum tragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Any material used in preparing any dosage unit form should be of pharmaceutically acceptable purity and substantially non-toxic in the amounts employed. In addition, the pharmaceutical composition can be incorporated into sustained-release preparations and formulations.

The pharmaceutical composition described herein may comprise one or more permeation enhancer that facilitates bioavailability of the polynucleotide molecule described herein. WO 2000/67798, Muranishi, 1990, Crit. Rev. Ther. Drug Carrier Systems, 7, 1, Lee et al., 1991, Crit Rev. Ther. Drug Carrier Systems, 8, 91 are herein incorporated by reference in its entirety. In some aspects, the permeation enhancer is intestinal. In some aspects, the permeation enhancer is transdermal. In some aspects, the permeation enhancer is to facilitate crossing the brain-blood barrier. In some aspects, the permeation enhancer improves the permeability in the oral, nasal, buccal, pulmonary, vaginal, or corneal delivery model. In some aspects, the permeation enhancer is a fatty acid or a derivative thereof. In some aspects, the permeation enhancer is a surfactant or a derivative thereof. In some aspects, the permeation enhancer is a bile salt or a derivative thereof. In some aspects, the permeation enhancer is a chelating agent or a derivative thereof. In some aspects, the permeation enhancer is a non-chelating non-surfactant or a derivative thereof. In some aspects, the permeation enhancer is an ester or a derivative thereof. In some aspects, the permeation enhancer is an ether or a derivative thereof. In some aspects, the permeation enhancer is arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof. In one specific aspect, the permeation enhancer is sodium caprate (C10). In some aspects, the permeation enhancer is chenodeoxycholic acid (CDCA), ursodeoxychenodeoxycholic acid (UDCA), cholic acid, dehydrocholic acid, deoxycholic acid, glucholic acid, glycholic acid, glycodeoxycholic acid, taurocholic acid taurodeoxycholic acid, sodium tauro-24,25-dihydro-fusidate or sodium glycodihydrofusidate. In some aspects, the permeation enhancer is polyoxyethylene-9-lauryl ether, or polyoxyethylene-20-cetyl ether.

For the polynucleotide molecule described herein, suitable pharmaceutically acceptable salts include (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, and the like; and (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like.

While the hybridized polynucleotide constructs described herein may not require the use of excipients for delivery to the target cell, the use of excipients may be advantageous in some aspects. Thus, for delivery to the target cell, the hybridized polynucleotide molecule described herein can non-covalently bind an excipient to form a complex. The excipient can be used to alter biodistribution after delivery, to enhance uptake, to increase half-life or stability of the strands in the hybridized polynucleotide constructs (e.g., improve nuclease resistance), and/or to increase targeting to a particular cell or tissue type.

Exemplary excipients include a condensing agent (e.g., an agent capable of attracting or binding a nucleic acid through ionic or electrostatic interactions); a fusogenic agent (e.g., an agent capable of fusing and/or being transported through a cell membrane); a protein to target a particular cell or tissue type (e.g., thyrotropin, melanotropin, lectin, glycoprotein, surfactant protein A, or any other protein); a lipid; a lipopolysaccharide; a lipid micelle or a liposome (e.g., formed from phospholipids, such as phosphotidylcholine, fatty acids, glycolipids, ceramides, glycerides, cholesterols, or any combination thereof); a nanoparticle (e.g., silica, lipid, carbohydrate, or other pharmaceutically-acceptable polymer nanoparticle); a polyplex formed from cationic polymers and an anionic agent (e.g., a CRO), where exemplary cationic polymers include poly amines (e.g., polylysine, polyarginine, polyamidoamine, and polyethylene imine); cholesterol; a dendrimer (e.g., a polyamidoamine (PAMAM) dendrimer); a serum protein (e.g., human serum albumin (HSA) or low-density lipoprotein (LDL)); a carbohydrate (e.g., dextran, pullulan, chitin, chitosan, inulin, cyclodextrin, or hyaluronic acid); a lipid; a synthetic polymer, (e.g., polylysine (PLL), polyethylenimine, poly-L-aspartic acid, poly-L-glutamic acid, styrene-maleic acid anhydride copolymer, poly(L-lactide-co-glycolic) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacrylic acid), N-isopropylacrylamide polymer, pseudopeptide-polyamine, peptidomimetic polyamine, or polyamine); a cationic moiety (e.g., cationic lipid, cationic porphyrin, quaternary salt of a polyamine, or alpha helical peptide); a multivalent sugar (e.g., multivalent lactose, multivalent galactose, N-acetyl-galactosamine, N-acetyl-glucosamine, multivalent mannose, or multivalent fucose); a vitamin (e.g., vitamin A, vitamin E, vitamin K, vitamin B, folic acid, vitamin B12, riboflavin, biotin, or pyridoxal); a cofactor; or a drug to disrupt cellular cytoskeleton to increase uptake (e.g., taxol, vincristine, vinblastine, cytochalasin, nocodazole, japlakinolide, latrunculin A, phalloidin, swinholide A, indanocine, or myoservin).

Other therapeutic agents as described herein may be included in a pharmaceutical composition described herein in combination with a polynucleotide molecule described herein.

### Methods of Treatment

In some aspects, described herein is a method of modulating mRNA expression of FXI gene in a subject, comprising: administering to the subject a polynucleic acid molecule described herein, a polynucleic acid molecule conjugate described herein, or a pharmaceutical composition described herein, thereby modulating the mRNA expression of FXI gene in the subject.

In some aspects, the method described herein reduces expression of FXI gene in a subject by about or at least 10% compared to a negative control. In some aspects, the method described herein reduces expression of FXI gene in a subject by about or at least 20% compared to a negative control. In some aspects, the method described herein reduces expression of FXI gene in a subject by about or at least 30% compared to a negative control. In some aspects, the method described herein reduces expression of FXI gene in a subject by about or at least 40% compared to a negative control. In some aspects, the method described herein reduces expression of FXI gene in a subject by about or at least 50% compared to a negative control. In some aspects, the method described herein reduces expression of FXI gene in a subject by about or at least 60% compared to a negative control. In some aspects, the method described herein reduces expression of FXI gene in a subject by about or at least 70% compared to a negative control. In some aspects, the method described herein reduces expression of FXI gene in a subject by about or at least 80% compared to a negative control. In some aspects, the method described herein reduces expression of 17 gene in a subject by about or at least 90% compared to a negative control. In some aspects, the method described herein reduces expression of FXI gene in a subject by about 100% compared to a negative control.

In some aspects, the method described herein achieves an IC50 value of about 5 nM. In some aspects, the method described herein achieves an IC50 value of about 10 nM. In some aspects, the method described herein achieves an IC50 value of about 15 nM. In some aspects, the method described herein achieves an IC50 value of about 20 nM. In some aspects, the method described herein achieves an IC50 value of about 25 nM. In some aspects, the method described herein achieves an IC50 value of about 30 nM. In some aspects, the method described herein achieves an IC50 value of about 35 nM. In some aspects, the method described herein achieves an IC50 value of about 40 nM. In some aspects, the method described herein achieves an IC50 value of about 45 nM. In some aspects, the method described herein achieves an IC50 value of about 50 nM. In some aspects, the method described herein achieves an IC50 value of about 55 nM. In some aspects, the method described herein achieves an IC50 value of about 60 nM. In some aspects, the method described herein achieves an IC50 value of about 65 nM. In some aspects, the method described herein achieves an IC50 value of about 70 nM. In some aspects, the method described herein achieves an IC50 value of about 75 nM. In some aspects, the method described herein achieves an IC50 value of about 80 nM. In some aspects, the method described herein achieves an IC50 value of about 85 nM. In some aspects, the method described herein achieves an IC50 value of about 90 nM. In some aspects, the method described herein achieves an IC50 value of about 95 nM. In some aspects, the method described herein achieves an IC50 value of about 100 nM.

In some aspects, the method described herein achieves an IC50 value of about 1 μM. In some aspects, the method described herein achieves an IC50 value of about 1.1 μM. In some aspects, the method described herein achieves an IC50 value of about 1.2 μM. In some aspects, the method described herein achieves an IC50 value of about 1.3 μM. In some aspects, the method described herein achieves an IC50 value of about 1.4 μM. In some aspects, the method described herein achieves an IC50 value of about 1.5 μM. In some aspects, the method described herein achieves an IC50 value of about 2 μM. In some aspects, the method described herein achieves an IC50 value of about 4 μM. In some aspects, the method described herein achieves an IC50 value of about 6 μM. In some aspects, the method described herein achieves an IC50 value of about 8 μM. In some aspects, the method described herein achieves an IC50 value of about 10 μM. In some aspects, the method described herein achieves an IC50 value of about 12 μM. In some aspects, the method described herein achieves an IC50 value of about 13 μM. In some aspects, the method described herein achieves an IC50 value of about 14 μM. In some aspects, the method described herein achieves an IC50 value of about 15 μM. In some aspects, the method described herein achieves an IC50 value of about 30 μM. In some aspects, the method described herein achieves an IC50 value of about 35 μM. In some aspects, the method described herein achieves an IC50 value of about 40 μM. In some aspects, the method described herein achieves an IC50 value of about 50 μM. In some aspects, the method described herein achieves an IC50 value of about 60 μM. In some aspects, the method described herein achieves an IC50 value of about 80 μM. In some aspects, the method described herein achieves an IC50 value of about 100 μM. In some aspects, the method described herein achieves an IC50 value of about 120 μM. In some aspects, the method described herein achieves an IC50 value of about 160 μM.

In some aspects, described herein is a method of modulating FXI or FXIa protein levels or FXI or FXIa activity in a subject in need thereof, comprising administering to the subject a polynucleic acid molecule described herein, a polynucleic acid molecule conjugate described herein, or a pharmaceutical composition described herein, wherein the polynucleic acid molecule described herein, the polynucleic acid molecule conjugate described herein, or the pharmaceutical composition described herein modulates the FXI or FXIa protein levels or FXI or FXIa activity in the subject.

In some another aspects, the method described herein reduces FXI or FXIa levels in a subject by about or at least 10% compared to a negative control. In some aspects, the method described herein reduces FXI or FXIa levels in a subject by about or at least 20% compared to a negative control. In some aspects, the method described herein reduces FXI or FXIa levels in a subject by about or at least 30% compared to a negative control. In some aspects, the method described herein reduces FXI or FXIa levels in a subject by about or at least 40% compared to a negative control. In some aspects, the method described herein reduces FXI or FXIa levels in a subject by about or at least 50% compared to a negative control. In some aspects, the method described herein reduces FXI or FXIa levels in a subject by about or at least 60% compared to a negative control. In some aspects, the method described herein reduces FXI or FXIa levels in a subject by about or at least 70% compared to a negative control. In some aspects, the method described herein reduces FXI or FXIa levels in a subject by about or at least 80% compared to a negative control. In some aspects, the method described herein reduces FXI or FXIa levels in a subject by about or at least 90% compared to a negative control. In some aspects, the method described herein reduces FXI or FXIa levels in a subject by about 100% compared to a negative control.

In some aspects, the subject receiving the method described herein suffers from thrombosis. In some case, the subject receiving the method described herein suffers from deep vein/venous thrombosis (DVT). In other aspects, the subject receiving the method described herein suffers from ischemic stroke. In other aspects, the subject receiving the method described herein suffers from atherosclerosis. In other aspects, the subject receiving the method described herein suffers from myocardial infarction. In other aspects, the subject receiving the method described herein suffers from venous thromboembolism (VTE). In other aspects, the subject receiving the method described herein suffers a cardiovascular disease. In other aspects, the subject receiving the method described herein suffers from ischemic heart disease (acute coronary syndrome). In other aspects, the subject receiving the method described herein suffers from pulmonary embolism.

### Exemplary Embodiments

Embodiment 1. A polynucleic acid molecule for modulating expression of coagulation factor XI (FXI) gene, wherein the polynucleic acid molecule comprises a nucleic acid sequence that is at least 80%, at least 85%, at least 90% identical to a nucleic acid sequence in Tables 1-2 and Tables 7-9

Embodiment 2. The polynucleic acid molecule of embodiment 1, wherein the polynucleic acid molecule is a single-stranded nucleic acid molecule

Embodiment 3. The polynucleic acid molecule of embodiment 2, wherein the single-stranded nucleic acid molecule comprises at least 14, 15, 16, 17, 18 consecutive nucleotides that are complementary to a nucleic acid sequence selected from SEQ ID NOs: 101-150 and 220, with no more than 1, 2, 3, 4 mismatches.

Embodiment 4. The polynucleic acid molecule of embodiment 2, wherein the single-stranded nucleic acid molecule comprises a sequence that is at least 80%, at least 85%, at least 90%, at least 95% complementary to a nucleic acid sequence selected from SEQ ID NOs: 101-150 and 220.

Embodiment 5. The polynucleic acid molecule of embodiment 2, wherein the single-stranded nucleic acid molecule comprises at least 14, 15, 16, 17, 18 consecutive nucleotides that are identical to a nucleic acid sequence selected from SEQ ID NOs: 1-50 and 215-216, with no more than 1, 2, 3, 4 mismatches.

Embodiment 6. The polynucleic acid molecule of embodiment 2, wherein the single-stranded nucleic acid molecule comprises a sequence that is at least 80%, at least 85%, at least 90%, at least 95% identical to a nucleic acid sequence selected from SEQ ID NOs: 1-50 and 215-216.

Embodiment 7. The polynucleic acid molecule of embodiment 1, wherein the polynucleic acid molecule is a double-stranded nucleic acid molecule comprising a sense strand (passenger strand) and an antisense strand (guide strand).

Embodiment 8. The polynucleic acid molecule of embodiment 7, wherein the sense strand comprises a nucleic acid sequence that is at least 80%, at least 85%, at least 90%, at least 95% identical to a nucleic acid sequence selected from SEQ ID NOs: 101-150 and 220.

Embodiment 9. The polynucleic acid molecule of any one of embodiments 7-8, wherein the antisense strand comprises a nucleic acid sequence that is at least 80%, at least 85%, at least 90%, at least 95% identical to a nucleic acid sequence selected from SEQ ID NOs: 1-50 and 215-216.

Embodiment 10. The polynucleic acid molecule of any one of embodiments 7-9, wherein the sense strand comprises a nucleic acid sequence comprising at least 14, 15, 16, 17, 18, 19, or 20 consecutive sequences of a nucleic acid sequence selected from SEQ ID NOs: 101-150 and 220 with no more than 1, 2, 3, or 4 mismatches.

Embodiment 11. The polynucleic acid molecule of any one of embodiments 7-10, wherein the antisense strand comprises a nucleic acid sequence comprising at least 14, 15, 16, 17, 18, 19, 20, 21, or 22 consecutive sequences of a nucleic acid sequence selected from SEQ ID NOs:1-50 and 215-216 with no more than 1, 2, 3, or 4 mismatches

Embodiment 12. The polynucleic acid molecule of any one of embodiments 7-11, wherein the sense strand comprises a nucleic acid sequence selected from SEQ ID NOs: 101-150 and 220 and the antisense strand comprises a nucleic acid sequence selected from SEQ ID NOs: 1-50 and 215-216

Embodiment 13. The polynucleic acid molecule of any one of embodiments 7-12, wherein the sense strand comprises a nucleic acid sequence that is at least 90%, at least 95% identical to a nucleic acid sequence selected from SEQ ID NOs: 104, 106-107, 109-111, 113-114, 135, 150, and 220

Embodiment 14. The polynucleic acid molecule of any one of embodiments 7-13, wherein the antisense strand comprises a nucleic acid sequence that is at least 90%, at least 95% identical to a nucleic acid sequence selected from SEQ ID NOs: 4, 6-7, 9-11, 13-14, 35, 50, and 215-216

Embodiment 15. The polynucleic acid molecule of any one of embodiments 7-14, wherein the sense strand comprises a nucleic acid sequence selected from SEQ ID NOs: 104, 106-107, 109-111, 113-114, 135, 150, and 220 and the antisense strand comprises a nucleic acid sequence selected from a nucleic acid sequence of SEQ ID NOs: 4, 6-7, 9-11, 13-14, 35, 50, and 215-216.

Embodiment 16. The polynucleic acid molecule of any one of embodiments 5-15, wherein the antisense strand comprises 5′-nNfnnnNfnNfNfnnnnNfnNfnnnnnnn-3′, 5′-nNfnnnNfnnnnnnnNfnNfnnnnnnn-3′, 5′-nNfnnnnNfnnnnNfnNfnnnnnnnnn-3′, 5′-nNfnnnnNfnnnnNfnNfnNfnnnnnnn-3′, or 5′-nNfnnnnnnnnnNfnNfnNfnnnnnnn-3′, wherein “Nf” stands for a 2′-fluoro modified nucleotide, and wherein “n” stands for a 2′-O-methyl modified nucleotide.

Embodiment 17. The polynucleic acid molecule of any one of embodiments 1-16, wherein the polynucleic acid molecule comprises (1) a 2′-fluoro modified nucleotides; (2) a 2′-O-methyl modified nucleotides; (3) 2′-deoxy modified nucleotides, or (4) a modified internucleotide linkage.

Embodiment 18. The polynucleic acid molecule of any one of embodiments 1-17, wherein the polynucleic acid molecule comprise at least two consecutive modified internucleotide linkages at the 5′ end and/or 3′ end.

Embodiment 19. The polynucleic acid molecule of any one of embodiments 7-18, wherein the antisense strand comprises at least two internucleotide linkages among 3 internucleotide linkages at the 3′end substituted with modified internucleotide linkages.

Embodiment 20. The polynucleic acid molecule of any one of embodiments 7-19, wherein the sense strand comprises 5′-nnnnnnNfnNfnNfnnnnnnnnnn-3′, 5′-nnnnnnNfnNfNfNfnnnnnnnnnn-3′, 5′-nnnnnnnnNfNfNfnnnnnnnnnn-3′, or 5′-nnnnnnNfnNfnNfnnnnnnnnnn-invdN-invdN-3′ wherein “Nf” stands for a 2′-fluoro modified nucleotide, and wherein “n” stands fora 2′-O-methyl modified nucleotide, and wherein “invdN” stands for an inverted deoxy-nucleotide.

Embodiment 21. The polynucleic acid molecule of any one of embodiments 7-19, wherein the sense strand comprises 5′-NfnNfnNfnNfnNfNfNfnNfnNfnNfnNfnNf-3′, wherein the antisense strand comprises 5′-nNfnNfnNfnNfnNfnnnNfnNfnNfnNfnnn-3′, wherein “Nf” stands fora 2′-fluoro modified nucleotide, and wherein “n” stands for a 2′-O-methyl modified nucleotide.

Embodiment 22. The polynucleic acid molecule of any one of embodiments 7-19, wherein the sense strand comprises 5′-nnnnnnNfnNfNfNfnnnnnnnnnn-3′, wherein the antisense strand comprises 5′-nNfnnnNfnNfNfnnnnNfnNfnnnnnnn-3′, wherein “Nf” stands for a 2′-fluoro modified nucleotide, and wherein “n” stands for a 2′-O-methyl modified nucleotide.

Embodiment 23. The polynucleic acid molecule of any one of embodiments 7-19, wherein the sense strand comprises 5′-nnnnnnnnNfnNfnnnnnnnnnn-3′, wherein the antisense strand comprises 5′-nNfnnnnnnnnnNfnNfnnnnnnnnn-3′, wherein “Nf” stands for a 2′-fluoro modified nucleotide, and wherein “n” stands for a 2′-O-methyl modified nucleotide.

Embodiment 24. The polynucleic acid molecule of any one of embodiments 7-19, wherein the sense strand comprises 5′-nnnnnnNfnNfnNfnnnnnnnnnn-3′, wherein the antisense strand comprises 5′-nNfnnnnnnnnnNfnNfnNfnnnnnnn-3′, wherein “Nf” stands for a 2′-fluoro modified nucleotide, and wherein “n” stands for a 2′-O-methyl modified nucleotide.

Embodiment 25. The polynucleic acid molecule of any one of embodiments 7-19, wherein the sense strand comprises 5′-nnnnnnNfnNfnNfnnnnnnnnnn-3′, wherein the antisense strand comprises 5′ _nNfnnnnNfnnnnNfnNfnNfnnnnnnn-3′, wherein “Nf” stands for a 2′-fluoro modified nucleotide, and wherein “n” stands for a 2′-O-methyl modified nucleotide.

Embodiment 26. The polynucleic acid molecule of any one of embodiments 17-25, wherein the modified internucleotide linkage is a phosphorothioate internucleotide linkage.

Embodiment 27. The polynucleic acid molecule of embodiment 26, wherein the modified internucleotide linkage comprises a stereochemically enriched phosphorothioate internucleotide linkage.

Embodiment 28. The polynucleic acid molecule of any one of embodiments 17-27, wherein the modified internucleotide linkage is an SP chiral internucleotide phosphorothioate linkage.

Embodiment 29. The polynucleic acid molecule of any one of embodiments 17-28, wherein the polynucleic acid comprises a plurality of modified internucleotide linkages, and at least 1, 2, 3, or 4 of the plurality of modified internucleotide linkages are stereochemically enriched phosphorothioate internucleotide linkages.

Embodiment 30. The polynucleic acid molecule of embodiment 29, wherein the stereochemically enriched phosphorothioate internucleotide linkages comprise both R- and S-isomers.

Embodiment 31. The polynucleic acid molecule of one of embodiments 29-30, wherein the stereochemically enriched phosphorothioate is disposed between two consecutive nucleosides that are two of six 5′ or 3′-terminal nucleosides of the sense strand or the antisense strand.

Embodiment 32. The polynucleic acid molecule of any one of embodiments 1-31, wherein the polynucleic acid molecule comprises a hypoxanthine nucleobase-containing nucleoside substitution.

Embodiment 33. The polynucleic acid molecule of embodiment 32, wherein the hypoxanthine nucleobase-containing nucleoside substitution is an inosine substitution.

Embodiment 34. The polynucleic acid molecule of embodiment 33, wherein the inosine substitution is within a seed region of the antisense strand.

Embodiment 35. The polynucleic acid molecule of any one of embodiments 33-34, wherein the inosine substitution is within 7 nucleotides from the 5′ end of the antisense strand.

Embodiment 36. The polynucleic acid molecule of any one of embodiments 33-35, wherein the inosine substitution is in the first nucleotide from the 5′ end of the antisense strand.

Embodiment 37. The polynucleic acid molecule of embodiment 33, wherein the inosine substitution comprises 2′-O-methylinosine-3′-phosphate.

Embodiment 38. The polynucleic acid molecule of any one of embodiments 1-37, wherein the first nucleotide from the 5′ end of the antisense strand is substituted by a uridine or an adenosine.

Embodiment 39. The polynucleic acid molecule of embodiment 38, wherein the uridine comprises 2′-O-methyluridine-3′-phosphate, or wherein the adenosine comprises 2′-O-methyl-8-bromo-adenosine-3′-phopshate.

Embodiment 40. The polynucleic acid molecule of any one of embodiments 1-39, wherein the polynucleic acid molecule comprises an abasic substitution.

Embodiment 41. The polynucleic acid molecule of embodiment 40, wherein the abasic substitution is at the 5^{th} or 7^{th} nucleotide from the 5′ end.

Embodiment 42. The polynucleic acid molecule of one of embodiments 1-41, wherein the cytotoxicity of the polynucleic acid molecule is decreased compared to unmodified polynucleic acid.

Embodiment 43. The polynucleic acid molecule of any one of embodiments 7-42, wherein the sense strand comprises a nucleic acid sequence that is at least 80%, at least 85%, at least 90%, at least 95% identical to a nucleic acid sequence selected from SEQ ID NOs: 151-200, 214, and 221.

Embodiment 44. The polynucleic acid molecule of any one of embodiments 7-43, wherein the antisense strand comprises a nucleic acid sequence that is at least 80%, at least 85%, at least 90%, at least 95% identical to a nucleic acid sequence selected from SEQ ID NOs: 51-100, 201-213, and 217-219.

Embodiment 45. The polynucleic acid molecule of any one of embodiments 7-44, wherein the sense strand comprises a nucleic acid sequence selected from a nucleic acid sequence of SEQ ID NOs: 151-200, 214, and 221 and the antisense strand comprises a nucleic acid sequence selected from a nucleic acid sequence of SEQ ID NOs: 51-100, 201-213, and 217-219.

Embodiment 46. The polynucleic acid molecule of any one of embodiment s 1-45, wherein the polynucleic acid molecule is 19-25 base pairs in length.

Embodiment 47. The polynucleic acid molecule of any one of embodiment s 1-46, wherein the polynucleic acid molecule is 21-23 base pairs in length.

Embodiment 48. A polynucleic acid molecule for modulating expression of coagulation factor XI (FXI) gene, wherein the polynucleic acid molecule comprises:
(a) an antisense strand comprising a nucleotide sequence selected from SEQ ID NOs: 4, 6-7,
9-11, 13-14, 35, 50, and 215-216, and a sense strand comprising a nucleotide sequence selected from SEQ ID NOs: 104, 106-107, 109-111, 113-114, 135, 150, or 220, or
(b) an antisense strand comprising a nucleotide sequence selected from SEQ ID NOs: 54, 56-57, 59-61, 63-64, 85, 100, 201-213, and 217-219 and a sense strand comprising a nucleotide sequence selected from SEQ ID NO: 154, 156-157, 159-161, 163-164, 185, 200, 214, and 221.

Embodiment 49. The polynucleic acid molecule of embodiment 48, wherein the polynucleic acid molecule comprise:
(a) an antisense strand comprising a nucleotide sequence of AUAAAUGUCUUUGUUGCAAGCGC (SEQ ID NO:11) and a sense strand comprising a nucleotide sequence of GCUUGCAACAAAGACAUUUAU (SEQ ID NO: 111);
(b) an antisense strand comprising a nucleotide sequence of AUGUCUUUGUUGCAAGCGCUUAU (SEQ ID NO: 9) and a sense strand comprising a nucleotide sequence of AAGCGCUUGCAACAAAGACAU (SEQ ID NO: 109);
(c) an antisense strand comprising a nucleotide sequence of AAUGUCUUUGUUGCAAGCGCUUA (SEQ ID NO: 10) and a sense strand comprising a nucleotide sequence of AGCGCUUGCAACAAAGACAUU (SEQ ID NO: 110);
(d) an antisense strand comprising a nucleotide sequence of UUAUAGUUUAUGCCCUUCAUGUC (SEQ ID NO: 13) and a sense strand comprising a nucleotide sequence of CAUGAAGGGCAUAAACUAUAA (SEQ ID NO: 113);
(e) an antisense strand comprising a nucleotide sequence of AUAGGUAAAAAACUGGCAGCGGA (SEQ ID NO: 35) and a sense strand comprising a nucleotide sequence of CGCUGCCAGUUUUUUACCUAU (SEQ ID NO: 135)
(f) an antisense strand comprising a nucleotide sequence of IUAAAUGUCUUUGUUGCAAGCGC (SEQ ID NO: 215) and a sense strand comprising a nucleotide sequence of GCUUGCAACAAAGACAUUUAU (SEQ ID NO: 111); or
(g) an antisense strand comprising a nucleotide sequence of UUAAAUGUCUUUGUUGCAAGCGC (SEQ ID NO: 216) and a sense strand comprising a nucleotide sequence of GCUUGCAACAAAGACAUUUAA (SEQ ID NO: 220).

Embodiment 50. A polynucleic acid molecule for modulating expression of coagulation factor XI (FXI) gene, wherein the polynucleic acid molecule comprise:
(a) an antisense strand comprising a nucleotide sequence of asUfsaaaugucuuUfgUfuGfcaagcsgsc (SEQ ID NO: 61) and a sense strand comprising a nucleotide sequence of gscsuugcAfaCfaAfagacauuuau (SEQ ID NO: 161);
(b) an antisense strand comprising a nucleotide sequence of asUfsaaaugucuuUfgUfuGfcsaagcsgsc (SEQ ID NO: 211) and a sense strand comprising a nucleotide sequence of gscsuugcAfaCfaAfagacauuuau (SEQ ID NO: 161);
(c) an antisense strand comprising a nucleotide sequence of asUfsaaaugucuuUfgUfsuGfcaagcsgsc (SEQ ID NO: 212) and a sense strand comprising a nucleotide sequence of gscsuugcAfaCfaAfagacauuuau (SEQ ID NO: 161);
(d) an antisense strand comprising a nucleotide sequence of asUfsaaaugucuuUfgUfuGfcaasgcsgsc (SEQ ID NO: 213) and a sense strand comprising a nucleotide sequence of gscsuugcAfaCfaAfagacauuuau (SEQ ID NO: 161);
(e) an antisense strand comprising a nucleotide sequence of asUfsaaaugucuuUfgUfuGfcaagcsgsc (SEQ ID NO: 61) and a sense strand comprising a nucleotide sequence of gscsuugcAfaCfaAfagacauuuau(invdT)(invdT) (SEQ ID NO: 214);
(f) an antisense strand comprising a nucleotide sequence of asUfsgucuUfuguuGfcAfaGfcgcuusasu (SEQ ID NO: 204) and a sense strand comprising a nucleotide sequence of asasgcgcUfuGfcAfacaaagacau (SEQ ID NO: 159);
(g) an antisense strand comprising a nucleotide sequence of asAfsugucUfuuguUfgCfaAfgcgcususa (SEQ ID NO: 205) and a sense strand comprising a nucleotide sequence of asgscgcuUfgCfaAfcaaagacauu (SEQ ID NO: 160);
(h) an antisense strand comprising a nucleotide sequence of asUfsaaauGfucuuUfgUfuGfcaagcsgsc (SEQ ID NO: 206) and a sense strand comprising a nucleotide sequence of gscsuugcAfaCfaAfagacauuuau (SEQ ID NO: 161);
(i) an antisense strand comprising a nucleotide sequence of usUfsauagUfuuauGfcCfcUfucaugsusc (SEQ ID NO: 207) and a sense strand comprising a nucleotide sequence of csasugaaGfgGfcAfuaaacuauaa (SEQ ID NO: 163);
(j) an antisense strand comprising a nucleotide sequence of asUfsagguAfaaaaAfcUfgGfcagcgsgsa (SEQ ID NO: 209) and a sense strand comprising a nucleotide sequence of csgscugcCfaGfuUfuuuuaccuau (SEQ ID NO: 185);
(k) an antisense strand comprising a nucleotide sequence of isUfsaaaugucuuUfgUfuGfcaagcsgsc (SEQ ID NO: 217) and a sense strand comprising a nucleotide sequence of gscsuugcAfaCfaAfagacauuuau (SEQ ID NO: 161);
(l) an antisense strand comprising a nucleotide sequence of usUfsaaauGfucuuUfgUfuGfcaagcsgsc (SEQ ID NO: 218) and a sense strand comprising a nucleotide sequence of gscsuugcAfaCfaAfagacauuuaa (SEQ ID NO: 222); or
(m) an antisense strand comprising a nucleotide sequence of a4sUfsaaauGfucuuUfgUfuGfcaagcsgsc (SEQ ID NO: 219) and a sense strand comprising a nucleotide sequence of gscsuugcAfaCfaAfagacauuuau (SEQ ID NO: 161);
wherein “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “invdT” refers to 3′ inverted thymidine, “i” refers to 2′-O-methylinosine-3′-phosphate, and “a4” refers to 2′-O-methyl-8-bromo-adenosine-3′-phopshate.

Embodiment 51. A polynucleic acid molecule conjugate for modulating expression of coagulation factor XI (FXI) gene, wherein the polynucleic acid molecule conjugate comprises a polynucleic acid molecule of any one of embodiments 1-50 and an asialoglycoprotein receptor targeting moiety.

Embodiment 52. The polynucleic acid molecule conjugate of embodiment 51, wherein the polynucleic acid molecule and the asialoglycoprotein receptor targeting moiety is coupled via a linker.

Embodiment 53. The polynucleic acid molecule conjugate of embodiment 52, wherein the linker comprises formula (IV) below,

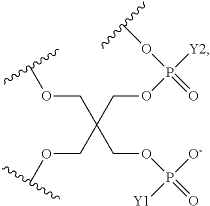

wherein at least one of Y1 and Y2 is a nucleotide in the polynucleic acid molecule.

Embodiment 54. The polynucleic acid molecule conjugate of embodiment 51, wherein the Y1 is the last nucleotide on the 3′-terminus of the sense strand of the polynucleic acid molecule, or wherein the Y2 is the first nucleotide on the 5′-terminus of the sense strand of the polynucleic acid molecule.

Embodiment 55. The polynucleic acid molecule conjugate of embodiment 54, wherein the Y1 and Y2 are two consecutive nucleotides in the polynucleic acid molecule.

Embodiment 56. The polynucleic acid molecule conjugate of any one of embodiments 51-55, wherein the asialoglycoprotein receptor targeting moiety comprises N-Acetylgalactosamine (GalNAc) or galactose.

Embodiment 57. The polynucleic acid molecule conjugate of any one of embodiments 51-56, wherein the linker and the asialoglycoprotein receptor targeting moiety with the last nucleotide on the 3′-terminus of the sense strand of the polynucleic acid molecule are shown in:

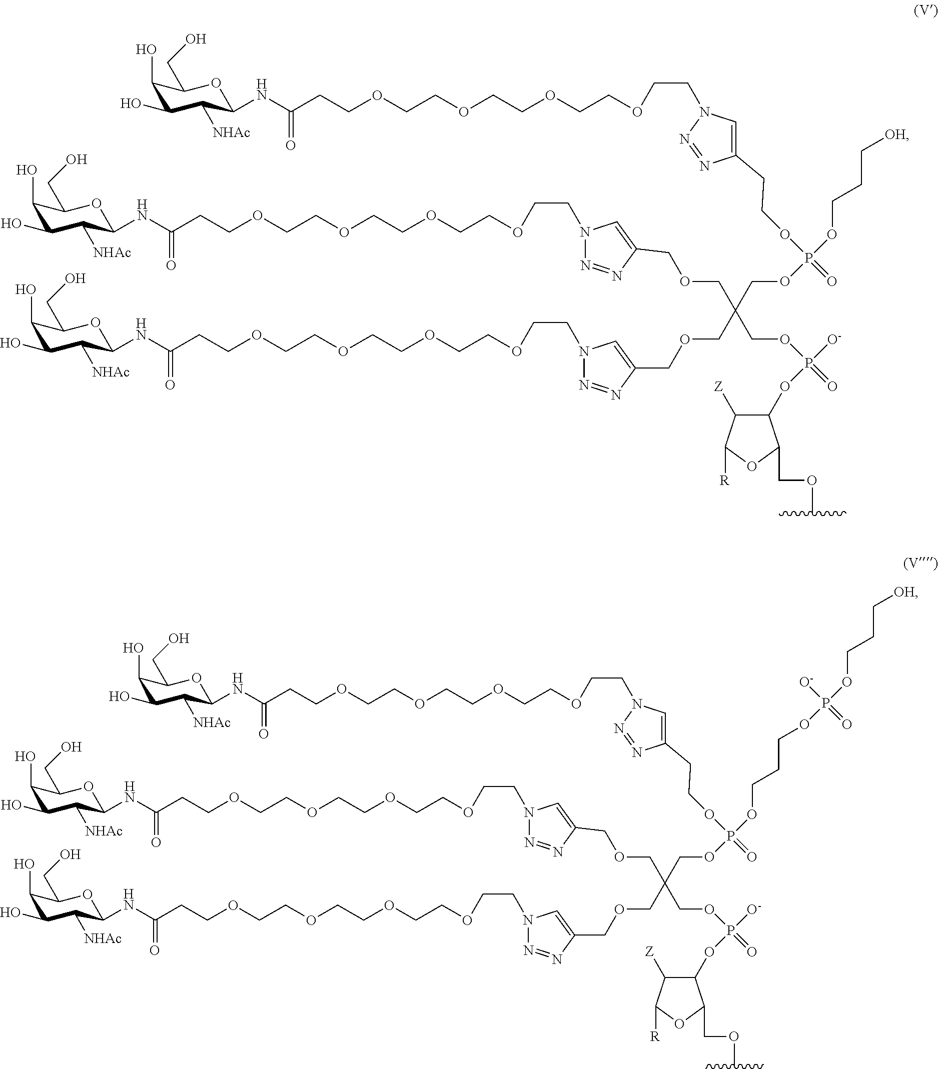

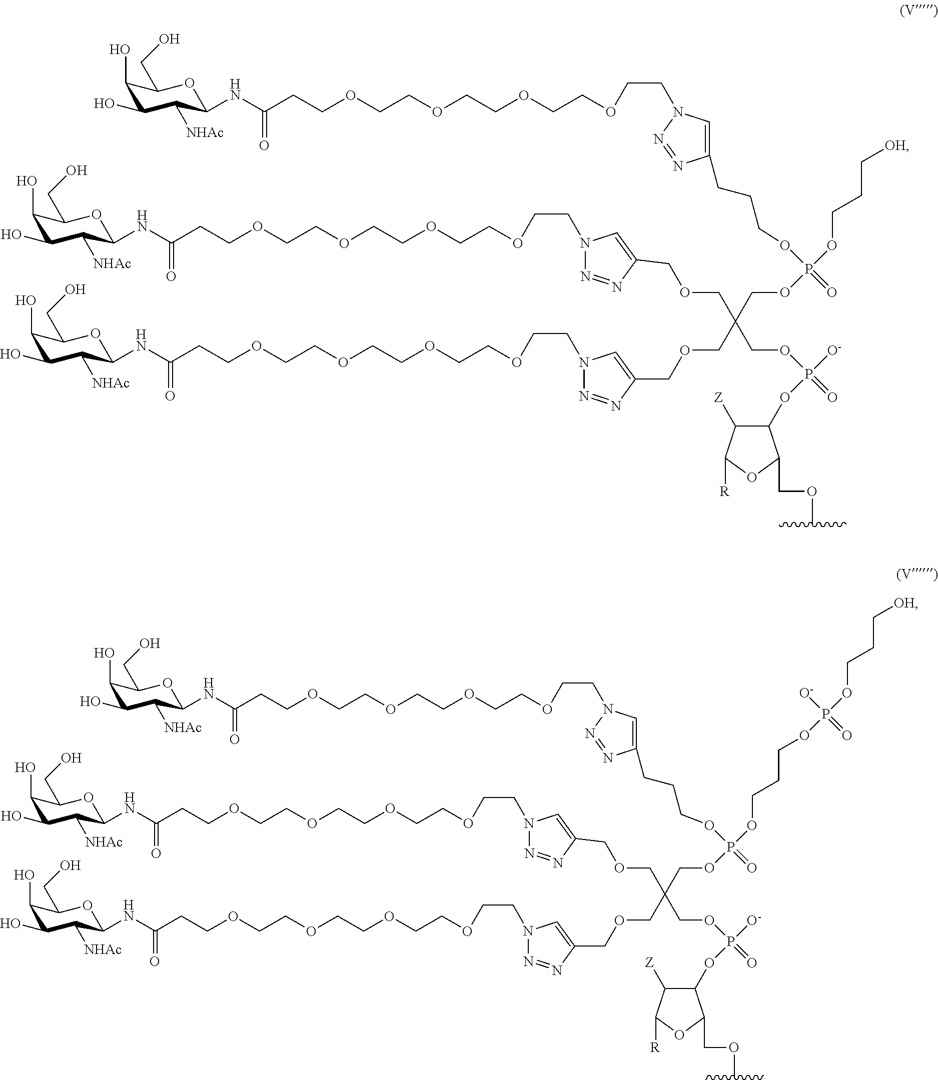

wherein Z in formula (V′), (V″″), (V″″′), or (V″″″) is —H, —OH, —O-Methyl, —F, or —O-methoxyethyl, and R in formula (V′) is adenine, uracil, guanine, cytosine, thymine, abasic, or others.

Embodiment 58. A pharmaceutical composition comprising a polynucleic acid molecule of any one of embodiments 1-57 or a polynucleic acid molecule conjugate of any one of embodiments 40-46, and a pharmaceutically acceptable excipient.

Embodiment 59. The pharmaceutical composition of embodiment 58, wherein the pharmaceutical composition is formulated as a nanoparticle formulation.

Embodiment 60. The pharmaceutical composition of embodiment 58 or 59, wherein the pharmaceutical composition is formulated for parenteral, oral, intranasal, buccal, rectal, transdermal, intravenous, subcutaneous, or intrathecal administration.

Embodiment 61. A method of modulating mRNA expression of coagulation factor XI (FXI) gene in a subject, comprising: administering to the subject a polynucleic acid molecule of any one of embodiments 1-57 or a polynucleic acid molecule conjugate of any one of embodiment s 40-46, or a pharmaceutical composition of embodiments 58-60, thereby modulating the mRNA expression of FXI gene in the subject.

Embodiment 62. A method of modulating FXI or FXIa protein levels or FXI or FXIa activity in a subject in need thereof, comprising: administering to the subject a polynucleic acid molecule of any one of embodiments 1-57 or a polynucleic acid molecule conjugate of any one of embodiments 40-46, or a pharmaceutical composition of embodiment s 58-60, thereby modulating the FXI or FXIa protein levels or FXI or FXIa activity in the subject.

Embodiment 63. The method of embodiment 62, wherein the subject in need thereof suffers from thrombosis or a symptom thereof.

### EXAMPLES

These examples are provided for illustrative purposes only and not to limit the scope of the claims provided herein. For all of the sequences presented herein, oligonucleotide structure representation reads from left to right (5′ to 3′). Monomer codes present in the oligonucleotide code are linked by 5′-3′ phosphodiester bonds unless specified (succeeded by 3′ internucleotide linkage reading left to right). Abbreviations of nucleotide monomers used in oligonucleotide structure representation are as follows. “A” stands for Adenosine-3′-phosphate; “a” stands for 2′-O-methyladenosine-3′-phosphate; “Af” stands for 2′-fluoroadenosine-3′-phosphate; “dA” stands for 2′-deoxyadenosine-3′-phosphate; “C” stands for Cytidine-3′-phosphate; “c” stands for 2′-O-methylcytidine-3′-phosphate; “Cf” stands for 2′-fluorocytidine-3′-phosphate; “dC” stands for 2′-deoxycytidine-3′-phosphate; “G” stands for Guanosine-3′-phosphate; “g” stands for 2′-O-methylguanosine-3′-phosphate; “Gf” stands for 2′-fluoroguanosine-3′-phosphate; “dG” stands for 2′-deoxyguanosine-3′-phosphate; “U” stands for Uridine-3′-phosphate; “u” stands for 2′-O-methyluridine-3′-phosphate; “Uf” stands for 2′-fluorouridine-3′-phosphate; “dU” stands for 2′-deoxyuridine-3′-phosphate; “T” stands for 5-methyluridine-3′-phosphate; “t” stands for 2′-O-methyl-5-methyluridine-3′-phosphate; “Tf” stands for 2′-fluoro-5-methyluridine-3′-phosphate; “dT” stands for thymidine-3′-phosphate; and “s” stands for 3′-phosphorothioate.

### Example 1—In Vitro Efficacy of siRNAs Targeting FXI

A panel of siRNAs were generated (shown in Table 1), and each passenger/sense strand was conjugated with a triantennary GalNAc moiety (GalNAc-L96). The siRNA-GalNAc conjugates were evaluated in vitro in primary human hepatocytes.

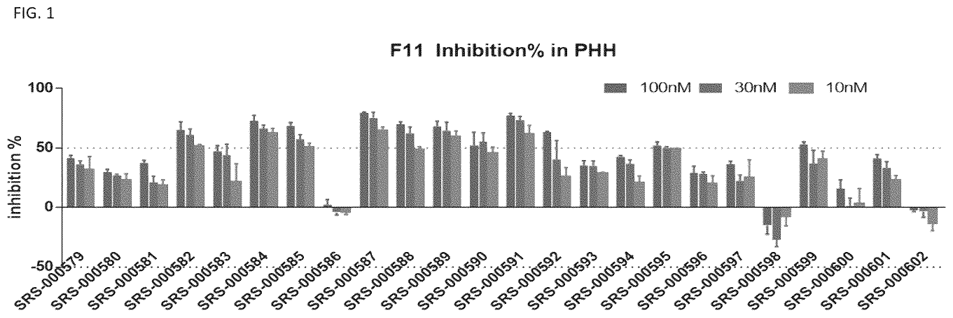

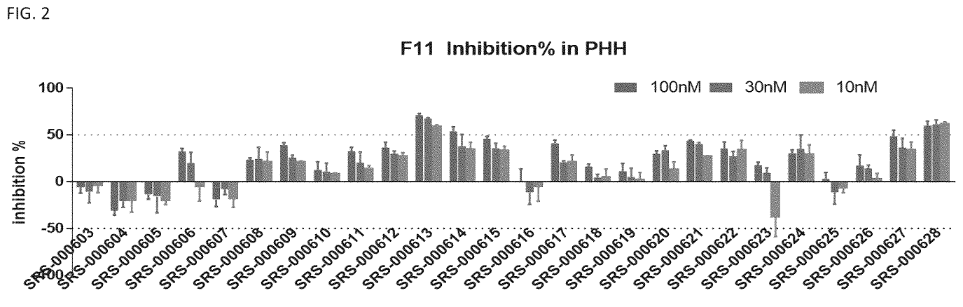

Cryopreserved primary human hepatocytes were thawed and plated on collagen-coated 96-well plates at a density of 5.4×10⁴ cells per well. Hepatocytes were treated by incubating with the siRNAs shown in Table 1 with each passenger/sense strand conjugated with a triantennary GalNAc moiety in the absence of transfection reagents (free uptake) for 48 hours. Cells were treated with the siRNAs at a concentrations of 100 nM, 30 nM, or 10 nM. At the end of the incubation period, the cells were lysed, the mRNA was isolated, and the expression of the target gene was measured by qPCR and normalized to a house-keeping gene, human GapDH, using standard protocols. 3 replicates were run per dose level per each siRNA. The in-vitro potency of the siRNAs are listed in Table 3. Results from Table 3 are plotted and shown in FIG. 1 and FIG. 2. Table 4 shows results from the positive control.

### Example 2—Drug Response Curves for Selected FXI siRNAs

A selected group of siRNAs targeting FXI shown in Table 2 were used, and each passenger/sense strand was conjugated with a triantennary GalNAc moiety via X2 linker (see Formula (V′)). For dose response curve determination, primary human hepatocytes were seeded into 96-well plates at appropriate density. siRNAs conjugated with 3′ sense strand X2-GalNAc (via formula V′) were added at the same time of seeding cells for free uptake, at 6 concentrations in triplicate. Concentrations of 1000, 100, 10, 1, 0.1, 0.01 nM were evaluated. At 48 hours after incubation, cells were harvested to measure FXI mRNA remaining by RT-PCR, and normalized to housekeeping gene (GAPDH). IC50 values and maximum FXI mRNA silencing was determined and the results are listed in Table 5.

### Example 3—In Vivo Efficacy of siRNA Targeting FXI in Cynomolgus Monkeys

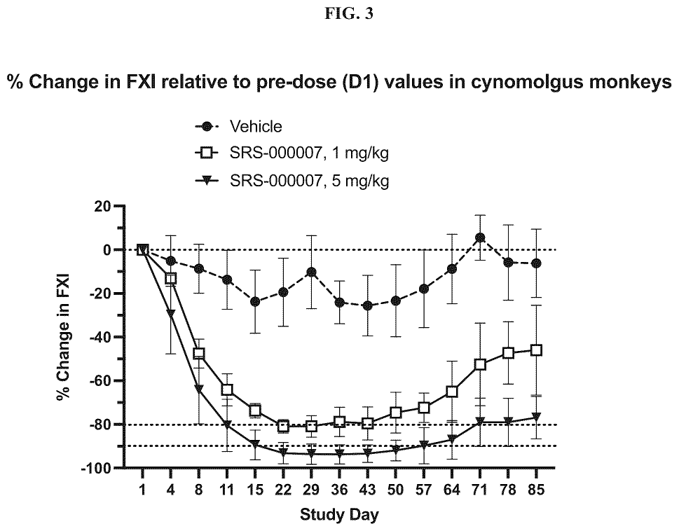

An siRNA targeting FXI, SRS-000007, shown in Table 2 was used, and its passenger/sense strand (SEQ ID NO: 161) was conjugated with a triantennary GalNAc moiety via X2 linker (see Formula (V′)) at the 3′ end. Female cynomolgus monkeys (n=4 per treatment group/siRNA) were administered with saline or SRS-000007 in a single subcutaneous dose level of 1 mg/kg or 5 mg/kg. Plasma samples were collected pre-dose (D7, D1), and on days 4, 8, 11, 15, 22, 29, 36, 43, 50, 57, 64, 71, 78, and 85 after treatment. FXI circulating protein levels in all plasma samples were analyzed using a FXI ELISA assay (Affinity Biologicals, product code FXI-AG). Results are shown in FIG. 3. The results are plotted as percent change in circulating FXI protein level relative to the pre-dose D1 timepoint.

### Example 4—In Vitro Dose Response in Primary Human Hepatocytes

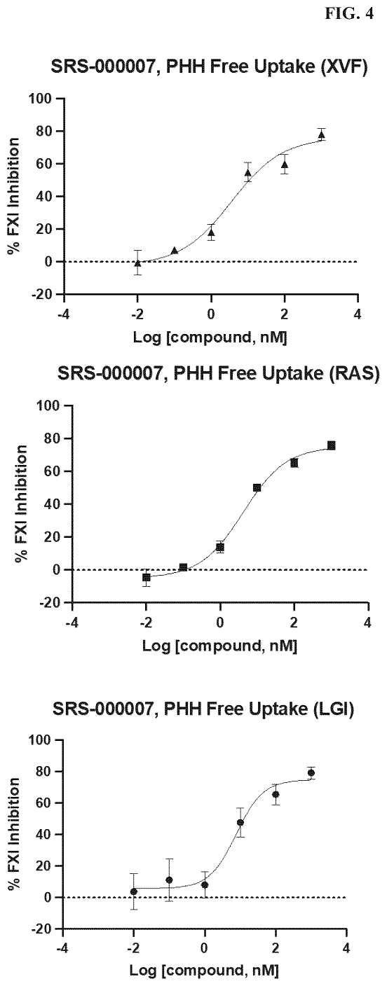

SRS-000007 targeting FXI as shown in Table 2 was used, and the passenger/sense strand was conjugated with a triantennary GalNAc moiety via X2 linker (see Formula (V′)). For dose response curve determination, three different donor lots of primary human hepatocytes were seeded into 96-well plates at appropriate density. SRS-000007 conjugated with 3′ sense strand X2-GalNAc (via formula V′) was added at the same time of seeding cells for free uptake, at 6 concentrations in triplicate. Concentrations of 1000, 100, 10, 1, 0.1, 0.01 nM were evaluated. At 72 hours after incubation, cells were harvested to measure FXI mRNA remaining by RT-PCR, and normalized to housekeeping gene (GAPDH). IC50 values and maximum FXI mRNA silencing was determined and the results are listed in Table 6. The dose response curves for each donor were plotted and are depicted in FIG. 4.

### Example 5—In Vivo Efficacy of siRNAs Targeting FXI in Cynomolgus Monkeys

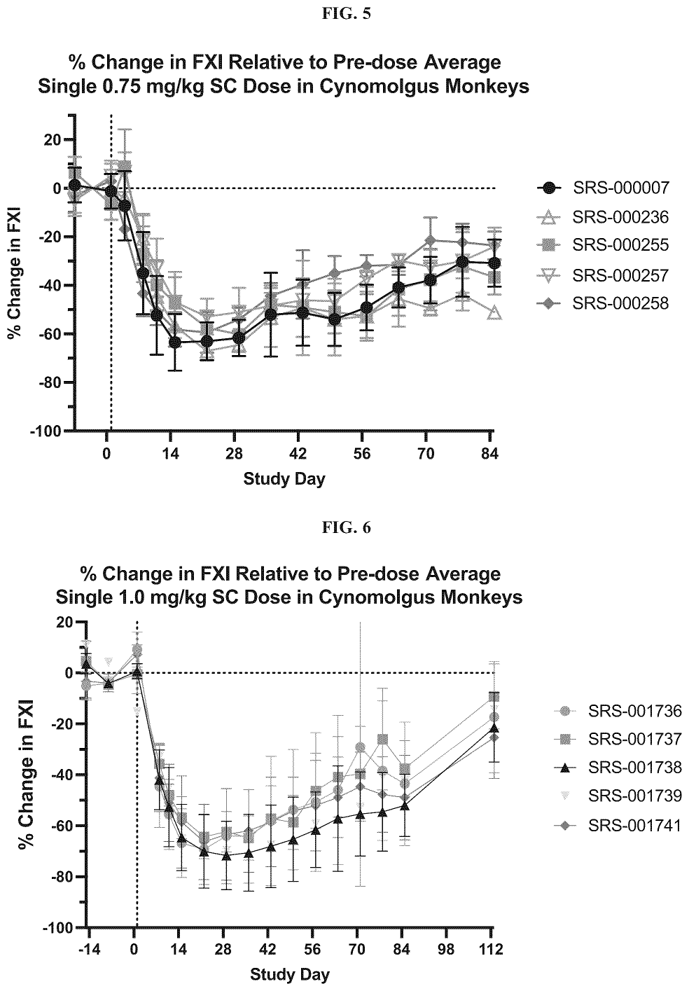

siRNAs targeting FXI shown in Table 7 were used. For siRNAs SRS-000007, SRS-000236, SRS-000255, and SRS-000257, each passenger/sense strand was conjugated with a triantennary GalNAc moiety via X2 linker (see Formula (V′)) at the 3′ end. For siRNA SRS-000258, the passenger/sense strand was conjugated with triantennary GalNAc moiety via X2 linker (see Formula (V″)) at the 5′ end. Female cynomolgus monkeys (n=3 per treatment group/siRNA) were administered in a single subcutaneous dose level of 0.75 mg/kg. Plasma samples were collected pre-dose (D-7, D1), and on days 4, 8, 11, 15, 22, 29, 36, 43, 50, 57, 64, 71, 78, and 85 after treatment. FXI circulating protein levels in all plasma samples were analyzed using a FXI ELISA assay (Affinity Biologicals, product code FXI-AG). Results are shown in FIG. 5. The results are plotted as percent change in circulating FXI protein level relative to the average pre-dose timepoints.

### Example 6—In Vivo Efficacy of siRNAs Targeting FXI in Cynomolgus Monkeys

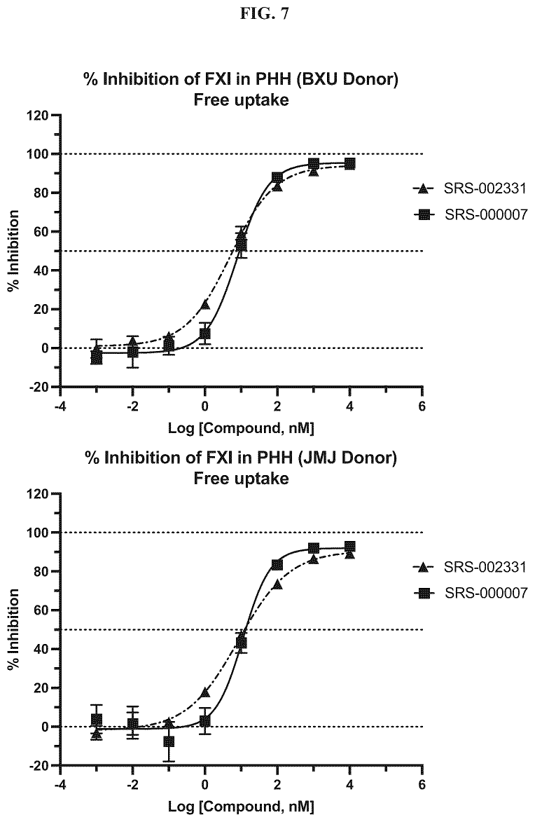

siRNAs targeting FXI shown in Table 8 were used. Each passenger/sense strand was conjugated with a triantennary GalNAc moiety via X2 linker (see Formula (V′)) at the 3′ end. Male cynomolgus monkeys (n=3 per treatment group/siRNA) were administered in a single subcutaneous dose level of 1.0 mg/kg. Plasma samples were collected pre-dose (D-15, D-8, and D1), and on days 8, 11, 15, 22, 29, 36, 43, 50, 57, 64, 71, 78, 85, and 113 after treatment. FXI circulating protein levels in all plasma samples were analyzed using a FXI ELISA assay (Affinity Biologicals, product code FXI-AG). Results are shown in FIG. 6. The results are plotted as percent change in circulating FXI protein level relative to the average pre-dose timepoints.

### Example 7—In Vitro Efficacy of siRNAs Targeting FXI

SRS-000007 and SRS-002331 from Table 9 were used, and the passenger/sense strand was conjugated with a triantennary GalNAc moiety via X2 linker (see Formula (V′)). For dose response curve determination, two different donor lots of primary human hepatocytes were seeded into 96-well plates at appropriate density. siRNAs were added at the same time of seeding cells for free uptake, at 8 concentrations in triplicate. Concentrations of 10000, 1000, 100, 10, 1, 0.1, 0.01, and 0.001 nM were evaluated. At 72 hours after incubation, cells were harvested to measure FXI mRNA remaining by RT-PCR, and normalized to housekeeping gene (GAPDH). IC50 values and maximum FXI mRNA silencing was determined and the results are listed in Table 10. The dose response curves for each donor were plotted and are depicted in FIG. 7.

### Example 8—In Vivo Efficacy of siRNAs Targeting FXI in Cynomolgus Monkeys

siRNAs targeting FXI shown in Table 9 are used. Each passenger/sense strand is conjugated with a triantennary GalNAc moiety via X2 linker (see Formula (V′)) at the 3′ end. Cynomolgus monkeys are administered subcutaneously. Plasma samples are collected pre-dose and different time points after treatment. FXI circulating protein levels in all plasma samples are analyzed using a FXI ELISA assay (Affinity Biologicals, product code FXI-AG), and compared to pre-dose levels.

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the disclosure. It is intended that the following claims define the scope of the disclosure and that methods and structures within the scope of these claims and their equivalents be covered thereby.

TABLE 1
Sequence Information for siRNAs Evaluated in vitro

| | Target | SEQ | | SEQ | | SEQ | | SEQ | |
|---|---|---|---|---|---|---|---|---|---|
| | Posi- | ID | Guide/Antisense | ID | Guide/Antisense | ID | Passenger/Sense Structure | ID | Passenger/Sense Base |
| Duplex ID | tion | NO. | Structure Code (5′-3′) | NO. | Base Sequence (5′-3′) | NO. | Code (5′-3′) | NO. | Sequence (5′-3′) |
| SRS-000579 | 154 | 51 | asGfsucacacauuCfaCfcAfgaaacsusg | 1 | AGUCACACAUUCACCAGAAACUG | 151 | gsusuucuGfgUfgAfaugugugacu | 101 | GUUUCUGGUGAAUGUGUGACU |
| SRS-000580 | 193 | 52 | asAfsuguccccucCfuUfcAfaagcasgsg | 2 | AAUGUCCCCUCCUUCAAAGCAGG | 152 | usgscuuuGfaAfgGfaggggacauu | 102 | UGCUUUGAAGGAGGGGACAUU |
| SRS-000581 | 358 | 53 | asUfsucacucuugGfcAfgUfguuucsusg | 3 | AUUCACUCUUGGCAGUGUUUCUG | 153 | gsasaacaCfuGfcCfaagagugaau | 103 | GAAACACUGCCAAGAGUGAAU |
| SRS-000582 | 389 | 54 | usGfsaaagaauacCfcAfgAfaauegscsu | 4 | UGAAAGAAUACCCAGAAAUCGCU | 154 | csgsauuuCfuGfcGfuauucuuuca | 104 | CGAUUUCUGGGUAUUCUUUCA |
| SRS-000583 | 394 | 55 | usUfsgcuugaaagAfaUfaCfccagasasa | 5 | UUGCUUGAAAGAAUACCCAGAAA | 155 | uscsugggUfaUfuCfuuucaagcaa | 105 | UCUGGGUAUUCUUUCAAGCAA |
| SRS-000584 | 395 | 56 | asUfsugcuugaaaGfaAfuAfcccagsasa | 6 | AUUGCUUGAAAGAAUACCCAGAA | 156 | csusggguAfuUfcUfuucaagcaau | 106 | CUGGGUAUUCUUUCAAGCAAU |
| SRS-000585 | 396 | 57 | usAfsuugcuugaaAfgAfaUfacccasgsa | 7 | UAUUGCUUGAAAGAAUACCCAGA | 157 | usgsgguaUfuCfulfucaagcaaua | 107 | UGGGUAUUCUUUCAAGCAAUA |
| SRS-000586 | 409 | 58 | usAfsuuuggugugAfgCfaUfugcuusgsa | 8 | UAUUUGGUGUGAGCAUUGCUUGA | 158 | asasgcaaUfgCfuCfacaccaasua | 108 | AAGCAAUGCUCACACCAAAUA |
| SRS-000587 | 429 | 59 | asUfsgucuuuguuGfcAfaGfcgcuusasu | 9 | AUGUCUUUGUUGCAAGCGCUUAU | 159 | asasgcgcUfuGfcAfacaaagacau | 109 | AAGCGCUUGCAACAAAGACAU |
| SRS-000588 | 430 | 60 | asAfsugucuuuguUfgCfaAfgcgcususa | 10 | AAUGUCUUUGUUGCAAGCGCUUA | 160 | asgscgcuUfgCfaAfcaaagacauu | 110 | AGCGCUUGCAACAAAGACAUU |
| SRS-000589 | 433 | 61 | asUfsaaaugucuuUfgUfuGfcaagcsgsc | 11 | AUAAAUGUCUUUGUUGCAAGCGC | 161 | gscsuugcAfaCfaAfagacauuuau | 111 | GCUUGCAACAAAGACAUUUAU |
| SRS-000590 | 435 | 62 | asCfsauaaaugucUfuUfgUfugcaasgsc | 12 | ACAUAAAUGUCUUUGUUGCAAGC | 162 | ususgcaaCfaAfaGfacauuuaugu | 112 | UUGCAACAAAGACAUUUAUGU |
| SRS-000591 | 465 | 63 | usUfsauaguuuauGfcCfcUfucaugsusc | 13 | UUAUAGUUUAUGCCCUUCAUGUC | 163 | csasugaaGfgGfcAfuaaacuauaa | 113 | CAUGAAGGGCAUAAACUAUAA |
| SRS-000592 | 503 | 64 | usUfsucuuggcauUfcUfuGfagcacsusc | 14 | UUUCUUGGCAUUCUUGAGCACUC | 164 | gsusgcucAfaGfaAfugccaagaaa | 114 | GUGCUCAAGAAUGCCAAGAAA |
| SRS-000593 | 583 | 65 | asCfsaaauguuacGfaUfgCfuccagsgsc | 15 | ACAAAUGUUACGAUGCUCCAGGC | 165 | csusggagCfaUfcGfuaacauuugu | 115 | CUGGAGCAUCGUAACAUUUGU |
| SRS-000594 | 589 | 66 | usAfsguagacaaaUfgUfuAfcgaugscsu | 16 | UAGUAGACAAAUGUUACGAUGCU | 166 | csasucguAfaCfaUfuugucuacua | 116 | CAUCGUAACAUUUGUCUACUA |
| SRS-000595 | 592 | 67 | usUfsucaguagacAfaAfuGfuuacgsasu | 17 | UUUCAGUAGACAAAUGUUACGAU | 167 | csgsuaacAfuUfuGfucuacugaaa | 117 | CGUAACAUUUGUCUACUGAAA |
| SRS-000596 | 663 | 68 | usAfsuuucagugaAfaAfuCfcagacsasc | 18 | UAUUUCAGUGAAAAUCCAGACAC | 168 | gsuscuggAfuUfuUfcacugaaaua | 118 | GUCUGGAUUUUCACUGAAAUA |
| SRS-000597 | 674 | 69 | asAfsagugcacagGfaUfuUfcagugsasa | 19 | AAAGUGCACAGGAUUUCAGUGAA | 169 | csascugaAfaUfcCfugugcacuuu | 119 | CACUGAAAUCCUGUGCACUUU |
| SRS-000598 | 689 | 70 | usAfscaagccagaUfuAfgAfaagugscsa | 20 | UACAAGCCAGAUUAGAAAGUGCA | 170 | csascuuuCfuAfaUfcuggcuugua | 120 | CACUUUCUAAUCUGGCUUGUA |
| SRS-000599 | 810 | 71 | asAfsgguaaaaaaCfaAfgCfaaccgsgsg | 21 | AAGGUAAAAAACAAGCAACCGGG | 171 | csgsguugCfuUfgUfuuuuuaccuu | 121 | CGGUUGCUUGUUUUUUACCUU |
| SRS-000600 | 844 | 72 | usCfsuuugagauuCfuUfuGfggccasusu | 22 | UCUUUGAGAUUCUUUGGGCCAUU | 172 | usgsgcccAfaAfgAfaucucaaaga | 122 | UGGCCCAAAGAAUCUCAAAGA |
| SRS-000601 | 856 | 73 | usAfsgacaaagauUfuCfuUfugagasusu | 23 | UAGACAAAGAUUUCUUUGAGAUU | 173 | uscsucaaAfgAfaAfucuuugucua | 123 | UCUCAAAGAAAUCUUUGUCUA |
| SRS-000602 | 865 | 74 | usGfsuuuuaaggaGfaCfaAfagauususc | 24 | UGUUUUAAGGAGACAAAGAUUUC | 174 | asasucuuUfgUfcUfccuuaaaaca | 124 | AAUCUUUGUCUCCUUAAAACA |
| SRS-000603 | 866 | 75 | asUfsguuuuaaggAfgAfcAfaagaususu | 25 | AUGUUUUAAGGAGACAAAGAUUU | 175 | asuscuuuGfuCfuCfcuuaaaacau | 125 | AUCUUUGUCUCCUUAAAACAU |
| SRS-000604 | 872 | 76 | usCfsucagauguuUfuAfaGfgagacsasa | 26 | UCUCAGAUGUUUUAAGGAGACAA | 176 | gsuscuccUfuAfaAfacaucugaga | 126 | GUCUCCUUAAAACAUCUGAGA |
| SRS-000605 | 902 | 77 | usCfsuuuuuaaugCfgUfgUfacuggsgsc | 27 | UCUUUUUAAUGCGUGUACUGGGC | 177 | cscsaguaCfaCfgCfauuaaaaaga | 127 | CCAGUACACGCAUUAAAAAGA |
| SRS-000606 | 923 | 78 | usGfsaaaccagaaAfgAfgCfuuugcsusc | 28 | UGAAACCAGAAAGAGCUUUGCUC | 178 | gscsaaagCfuCfuUfucugguuuca | 128 | GCAAAGCUCUUUCUGGUUUCA |
| SRS-000607 | 925 | 79 | asCfsugaaaccagAfaAfgAfgcuuusgsc | 29 | ACUGAAACCAGAAAGAGCUUUGC | 179 | asasagcuCfuUfuCfugguuucagu | 129 | AAAGCUCUUUCUGGUUUCAGU |
| SRS-000608 | 977 | 80 | usGfsuaaaaugaaGfaAfuGfgcagasasc | 30 | UGUAAAAUGAAGAAUGGCAGAAC | 180 | uscsugccAfuUfcUfucauuuuaca | 130 | UCUGCCAUUCUUCAUUUUACA |
| SRS-000609 | 994 | 81 | usAfsagaaaucagUfgUfcAfugguasasa | 31 | UAAGAAAUCAGUGUCAUGGUAAA | 181 | usasccauGfaCfaCfugauuucuua | 131 | UACCAUGACACUGAUUUCUUA |
| SRS-000610 | 1011 | 82 | asUfsauccaguucUfuCfuCfccaagsasa | 32 | AUAUCCAGUUCUUCUCCCAAGAA | 182 | csusugggAfgAfaGfaacuggauau | 132 | CUUGGGAGAAGAACUGGAUAU |
| SRS-000611 | 1018 | 83 | asGfscaacaauauCfcAfgUfucuucsusc | 33 | AGCAACAAUAUCCAGUUCUUCUC | 183 | gsasagaaCfuGfgAfuauuguugcu | 133 | GAAGAACUGGAUAUUGUUGCU |
| SRS-000612 | 1058 | 84 | usAfsuuggugcacAfgUfuUfcuggcsasg | 34 | UAUUGGUGCACAGUUUCUGGCAG | 184 | gscscagaAfaCfuGfugcaccaaua | 134 | GCCAGAAACUGUGCACCAAUA |
| SRS-000613 | 1084 | 85 | asUfsagguaaaaaAfcUfpGfcagcgscsa | 35 | AUAGGUAAAAAACUGGCAGCGGA | 185 | csgscugcCfaGfuUfuuuuaccuau | 135 | CGCUGCCAGUUUUUUACCUAU |
| SRS-000614 | 1145 | 86 | usUfsgaagaaagcUfuUfaAfguaacsasc | 36 | UUGAAGAAAGCUUUAAGUAACAC | 186 | gsusuacuUfaAfaGfcuuucuucaa | 136 | GUUACUUAAAGCUUUCUUCAA |
| SRS-000615 | 1202 | 87 | usUfsaauguguauCfcAfgAfgaugcscsu | 37 | UUAAUGUGUAUCCAGAGAUGCCU | 187 | gscsaucuCfuGfgAfuacacauuaa | 137 | GCAUCUCUGGAUACACAUUAA |
| SRS-000616 | 1241 | 88 | usGfsauuuuggugGfuAfcAfcucaususa | 38 | UGAUUUUGGUGGUACACUCAUUA | 188 | asusgaguGfuAfcCfaccaaaauca | 138 | AUGAGUGUACCACCAAAAUCA |
| SRS-000617 | 1242 | 89 | usUfsgauuuugguGfgUfaCfacucasusu | 39 | UUGAUUUUGGUGGUACACUCAUU | 189 | usgsagugUfaCfcAfccaasaucaa | 139 | UGAGUGUACCACCAAAAUCAA |
| SRS-000618 | 1356 | 90 | usUfsuccaaugauGfgAfgCfcuccascsa | 40 | UUUCCAAUGAUGGAGCCUCCACA | 190 | usgsgaggCfuCfcAfucauuggaaa | 140 | UGGAGGCUCCAUCAUUGGAAA |
| SRS-000619 | 1413 | 91 | asAfsaaucuuaggUfgAfcUfcuaccscsc | 41 | AAAAUCUUAGGUGACUCUACCCC | 191 | gsgsuagaGfuCfaCfcuaagauuuu | 141 | GGUAGAGUCACCUAAGAUUUU |
| SRS-000620 | 1446 | 92 | usCfsagauugauuUfaAfaAfugccascsu | 42 | UCAGAUUGAUUUAAAAUGCCACU | 192 | usgsgcauUfuUfaAfaucaaucuga | 142 | UGGCAUUUUAAAUCAAUCUGA |
| SRS-000621 | 1540 | 93 | usAfsacaaggcaaUfaUfcAfuacccsgsc | 43 | UAACAAGGCAAUAUCAUACCCGC | 193 | gsgsguauGfaUfaUfugccuuguua | 143 | GGGUAUGAUAUUGCCUUGUUA |
| SRS-000622 | 1566 | 94 | usUfsguaauucacUfgUfgGfuuuccsasg | 44 | UUGUAAUUCACUGUGGUUUCCAG | 194 | gsgsaaacCfaCfaGfugaauuacaa | 144 | GGAAACCACAGUGAAUUACAA |
| SRS-000623 | 1573 | 95 | asGfsaaucuguguAfaUfuCfacugusgsg | 45 | AGAAUCUGUGUAAUUCACUGUGG | 195 | ascsagugAfaUfuAfcacagauucu | 145 | ACAGUGAAUUACACAGAUUCU |
| SRS-000624 | 1576 | 96 | usUfsgagaaucugUfgUfaAfuucacsusg | 46 | UUGAGAAUCUGUGUAAUUCACUG | 196 | gsusgaauUfaCfaCfagauucucaa | 146 | GUGAAUUACACAGAUUCUCAA |
| SRS-000625 | 1699 | 97 | usAfsucuuggcuuUfcUfgGfagagusasu | 47 | UAUCUUGGCUUUCUGGAGAGUAU | 197 | ascsucucCfaGfaAfagccaagaua | 147 | ACUCUCCAGAAAGCCAAGAUA |
| SRS-000626 | 1707 | 98 | asCfsuaaggguauCfuUfgGfcuuucsusg | 48 | ACUAAGGGUAUCUUGGCUUUCUG | 198 | gsasaagcCfaAfgAfuacccuuagu | 148 | GAAAGCCAAGAUACCCUUAGU |
| SRS-000627 | 1745 | 99 | usAfsuguccucugUfaUfcUfcuucusgsg | 49 | UAUGUCCUCUGUAUCUCUUCUGG | 199 | asgsaagaGfaUfaCfagaggacaua | 149 | AGAAGAGAUACAGAGGACAUA |
| SRS-000628 | 1757 | 100 | usAfsuggguuauuUfuAfuGfuccucsusg | 50 | UAUGGGUUAUUUUAUGUCCUCUG | 200 | gsasggacAfuAfaAfauaacccaua | 150 | GAGGACAUAAAAUAACCCAUA |

Note:
the target position is relative to human transcript NM_000128.4; the corresponding sequence of a certain SEQ ID NO. is located on its right column of the same row. “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate, “Tf” refers to 2′-fluoro-5-methyluridine-3′-phosphate; “dT” refers to 2′-deoxythymidine-3′-phosphate; “s” refers to 3′-phosphorothioate; The “Target Position in NM_000128.4” is defined as the 5′ position of a 21-mer target site in human transcript (NCBI Reference Sequence No: NM_000128.4). Note that, in some instances, the sense strand sequence is coupled to a targeting moiety (e.g., GalNAc or galactose (e.g., L96) at the 3′ end of the sense strand.

TABLE 2
Sequence Information for siRNAs Evaluated for their Drug Response Curves

| | Target | SEQ | | SEQ | | SEQ | | SEQ | |
|---|---|---|---|---|---|---|---|---|---|
| | Posi- | ID | Guide/Antisense Structure Code | ID | Guide/Antisense Base | ID | Passenger/Sense Structure | ID | Passenger/Sense Base |
| Duplex ID | tion | NO. | (5′-3′) | NO. | Sequence (5′-3′) | NO. | Code (5′-3′) | NO. | Sequence (5′-3′) |
| SRS-001724 | 389 | 54 | usGfsaaagaauacCfcAfgAfaaucgscsu | 4 | UGAAAGAAUACCCAGAAAUCGCU | 154 | csgsacuuCfuGfgGfuauucuuuca | 104 | CGAUUUCUGOGUAUUCUUUCA |
| SRS-001733 | 389 | 201 | usGfsaaagAfauacCfcAfgAfaaucgscsu | 4 | UGAAAGAAUACCCAGAAAUCGCU | 154 | csgsauuuCfuGfgGfuauucuuuca | 104 | CGAUUUCUGGGUAUUCUUUCA |
| SRS-001725 | 395 | 56 | asUfsugcuugaaaGfaAfuAfcccaagsasa | 6 | AUUGCUUGAAAGAAUACCCAGAA | 156 | csusggguAfuUfcUfuucaagcaau | 106 | CUGGGUAUUCUUUCAAGCAAU |
| SRS-001734 | 395 | 202 | asUfsugcuUfgaaaGfaAfuAfcccagsasa | 6 | AUUGCUUGAAAGAAUACCCAGAA | 156 | csusggguAfuUfcUfuucaagcaau | 106 | CUGGGUAUUCUUUCAAGCAAU |
| SRS-001726 | 396 | 57 | usAfsuugcuugaaAfgAfaUfacccasgsa | 7 | UAUUGCUUGAAAGAAUACCCAGA | 157 | uscsgguaUfuCfuUfucaagcaaua | 107 | UGGGUAUUCUUUCAAGCAAU |
| | | | | | | | | | A |
| SRS-001735 | 396 | 203 | usAfsuugcUfugaaAfgAfaUfacccasgsa | 7 | UAUUGCUUGAAAGAAUACCCAGA | 157 | usgsgguaUfuCfuUfucaagcaaua | 107 | UGGGUAUUCUUUCAAGCAAU |
| | | | | | | | | | A |
| SRS-001727 | 429 | 59 | asUfsgucuuuguuGfcAfaGfcgcuusasu | 9 | AUGUCUUUGUUGCAAGCGCUUAU | 159 | asasgcgcUfuGfcAfacaaagacau | 109 | AAGCGCUUGCAACAAAGACAU |
| SRS-001736 | 429 | 204 | asUfsgucuUfuguuGfcAfaGfcgcuusasu | 9 | AUGUCUUUGUUGCAAGCGCUUAU | 159 | asasgcgcUfuGfcAfacaaagacau | 109 | AAGCGCUUGCAACAAAGACAU |
| SRS-001728 | 430 | 60 | asAfsugucuuuguUfgCfaAfgcgcususa | 10 | AAUGUCUUUGUUGCAAGCGCUUA | 160 | asgscgcuUfgCfaAfcaaagacauu | 110 | AGCGCUUGCAACAAAGACAUU |
| SRS-001737 | 430 | 205 | asAfsugucUfuuguUfgCfaAfgcgcususa | 10 | AAUGUCUUUGUUGCAAGCGCUUA | 160 | asgscgcuUfgCfaAfcaaagacauu | 110 | AGCGCUUGCAACAAAGACAUU |
| SRS-000007 | 433 | 61 | asUfsaaaugucuuUfgUfuGfcaagcsgsc | 11 | AUAAAUGUCUUUGUUGCAAGCGC | 161 | gscsuugcAfaCfaAfagacauuuau | 111 | GCUUGCAACAAAGACAUUUAU |
| SRS-001738 | 433 | 206 | asUfsaaauGfucuuUfgUfuGfcaagcsgsc | 11 | AUAAAUGUCUUUGUUGCAAGCGC | 161 | gscsuugcAfaCfaAfagacauuuau | 111 | GCUUGCAACAAAGACAUUUAU |
| SRS-001729 | 465 | 63 | usUfsauaguuuauGfcCfcUfucaugsusc | 13 | UUAUAGUUUAUGCCCUUCAUGUC | 163 | csasugaaGfgGfcAfuaaacuauaa | 113 | CAUGAAGGGCAUAAACUAUA |
| | | | | | | | | | A |
| SRS-001739 | 465 | 207 | usUfsauagUfuuauGfcCfcUfucaugsusc | 13 | UUAUAGUUUAUGCCCUUCAUGUC | 163 | csasugaaGfcGfcAfuaaacuauaa | 113 | CAUGAAGGGCAUAAACUAUA |
| | | | | | | | | | A |
| SRS-001730 | 503 | 64 | usUfsucuuggcauUfcUfuGfagcacsusc | 14 | UUUCUUGGCAUUCUUGAGCACUC | 164 | gsusgcucAfaGfaAfugccaagaaa | 114 | GUGCUCAAGAAUGCCAAGAAA |
| SRS-001740 | 503 | 208 | usUfsucuuGfgcauUfcUfuGfagcacsusc | 14 | UUUCUUGGCAUUCUUGAGCACUC | 164 | gsusgcucAfaGfaAfugccaagaaa | 114 | GUGCUCAAGAAUGCCAAGAAA |
| SRS-001731 | 1084 | 85 | asUfsagguaaaaaAfcUfgGgcagcgsgsa | 35 | AUAGGUAAAAAACUGGCAGCGGA | 185 | csgscugcCfaGfuUfuuuuaccuau | 135 | CGCUGCCAGUUUUUUACCUAU |
| SRS-001741 | 1084 | 209 | asUfsagguAfaaaaAfcUfgGfcagcgsgsa | 35 | AUAGGUAAAAAACUGGCAGCGGA | 185 | csgscugcCfaGfuUfuuuuaccuau | 135 | CGCUGCCAGUUUUUUACCUAU |
| SRS-001732 | 1757 | 100 | usAfsuggguuauuUfuAfuGfuccucsusg | 50 | UAUGGGUUAUUUUAUGUCCUCUG | 200 | csasggacAfuAfaAfauaacccaua | 150 | GAGGACAUAAAAUAACCCAUA |
| SRS-001742 | 1757 | 210 | usAfsugggUfuauuUfuAfuGfuccucsusg | 50 | DAUGGGUUAUUUUAUGUCCUCUG | 200 | gsasggacAfuAfaAfauaacccaua | 150 | GAGGACAUAAAAUAACCCAUA |

Note:
the target position is relative to human transcript NM_000128.4; the corresponding sequence of a certain SEQ ID NO. is located on its right column of the same row. “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate, “c” refers to 2′-O-methylcytidine-3′-phosphate; ″Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT″ refers to 2′-deoxythymidine-3′-phosphate; “s” refers to 3′-phosphorothioate; Note that, in some instances, the passenger/sense strand sequence of SEQ ID NO: 154, 156-157, 159-161, 163-164, 185, and 200 in this table is further coupled to X2-GalNAe moiety, e. g., Formula (V), at the 3′ end of the sense strand. In some specific instances, the passenger/sense strand sequence of SEQ ID NO: 154, 156-157, 159-161, 163-164, 185, and 200 in this table is further coupled to a GalNAc moiety via Formula (V′), (V″), (V″), or (V″), at the 3′ end of the sense strand.

TABLE 3
Results of In vitro efficacy of siRNAs targeting FXI

| No. | Compound | | | | | | |
|---|---|---|---|---|---|---|---|
| line | ID (FXD) | Average Inhibition % | | | SD | | |
| item | SIRIUS ID | 100 nM | 30 nM | 10 nM | 100 nM | 30 nM | 10 nM |
| 1 | SRS-000579 | 41.86 | 36.23 | 31.63 | 3.64 | 2.98 | 11.26 |
| 2 | SRS-000580 | 30.06 | 27.17 | 23.34 | 3.64 | 0.93 | 5.16 |
| 3 | SRS-000581 | 37.50 | 21.03 | 18.77 | 3.86 | 5.38 | 4.61 |
| 4 | SRS-000582 | 65.36 | 61.00 | 51.72 | 11.63 | 5.10 | 1.37 |
| 5 | SRS-000583 | 47.26 | 44.11 | 21.47 | 8.23 | 9.05 | 15.39 |
| 6 | SRS-000584 | 73.07 | 66.41 | 62.69 | 7.68 | 2.86 | 3.83 |
| 7 | SRS-000585 | 68.84 | 57.50 | 51.26 | 4.59 | 3.76 | 2.96 |
| 8 | SRS-000586 | 2.50 | −4.13 | −3.96 | 7.64 | 2.14 | 1.81 |
| 9 | SRS-000587 | 79.57 | 75.30 | 65.21 | 1.16 | 4.75 | 2.43 |
| 10 | SRS-000588 | 70.10 | 62.29 | 49.10 | 3.41 | 5.42 | 2.16 |
| 11 | SRS-000589 | 68.19 | 64.62 | 59.98 | 7.61 | 7.22 | 4.45 |
| 12 | SRS-000590 | 52.23 | 55.45 | 45.62 | 19.11 | 7.29 | 5.16 |
| 13 | SRS-000591 | 77.36 | 73.40 | 61.96 | 3.21 | 3.12 | 7.24 |
| 14 | SRS-000592 | 63.58 | 40.59 | 25.86 | 0.61 | 15.74 | 7.89 |
| 15 | SRS-000593 | 35.31 | 35.14 | 29.08 | 7.10 | 3.99 | 0.65 |
| 16 | SRS-000594 | 42.64 | 36.87 | 20.86 | 2.01 | 3.24 | 5.72 |
| 17 | SRS-000595 | 52.13 | 49.93 | 49.24 | 5.29 | 1.33 | 0.60 |
| 18 | SRS-000596 | 29.23 | 28.43 | 20.32 | 9.70 | 1.57 | 6.39 |
| 19 | SRS-000597 | 36.59 | 22.47 | 25.62 | 4.29 | 4.81 | 14.41 |
| 20 | SRS-000598 | −15.08 | −27.09 | −7.38 | 12.62 | 5.76 | 8.04 |
| 21 | SRS-000599 | 53.03 | 36.99 | 40.71 | 3.73 | 11.11 | 6.63 |
| 22 | SRS-000600 | 16.01 | 1.58 | 3.81 | 12.54 | 6.32 | 12.25 |
| 23 | SRS-000601 | 41.37 | 33.31 | 23.07 | 5.56 | 5.29 | 3.85 |
| 24 | SRS-000602 | −2.38 | −3.26 | −13.38 | 1.66 | 4.92 | 6.00 |
| 25 | SRS-000603 | −6.27 | −11.07 | −3.37 | 10.31 | 11.58 | 8.36 |
| 26 | SRS-000604 | −30.88 | −21.15 | −20.30 | 8.15 | 6.22 | 12.26 |
| 27 | SRS-000605 | −13.83 | −15.85 | −20.24 | 8.54 | 17.42 | 4.22 |
| 28 | SRS-000606 | 32.37 | 20.09 | −5.43 | 5.53 | 11.35 | 15.13 |
| 29 | SRS-000607 | −19.27 | −8.09 | −17.91 | 12.81 | 5.42 | 9.48 |
| 30 | SRS-000608 | 23.59 | 24.52 | 21.21 | 3.12 | 12.18 | 10.57 |
| 31 | SRS-000609 | 39.04 | 25.72 | 21.67 | 4.69 | 2.53 | 0.53 |
| 32 | SRS-000610 | 12.50 | 11.09 | 8.42 | 15.08 | 8.75 | 1.00 |
| 33 | SRS-000611 | 32.58 | 20.47 | 13.64 | 7.10 | 11.32 | 4.02 |
| 34 | SRS-000612 | 36.68 | 30.06 | 27.52 | 9.64 | 2.64 | 3.61 |
| 35 | SRS-000613 | 71.02 | 67.64 | 59.10 | 3.14 | 0.72 | 1.52 |
| 36 | SRS-000614 | 53.88 | 38.01 | 34.62 | 8.21 | 12.62 | 7.66 |
| 37 | SRS-000615 | 46.18 | 35.76 | 33.76 | 3.80 | 5.24 | 4.15 |
| 38 | SRS-000616 | 0.06 | −11.80 | −4.95 | 23.52 | 12.43 | 15.73 |
| 39 | SRS-000617 | 41.04 | 20.78 | 21.24 | 5.72 | 1.65 | 7.40 |
| 40 | SRS-000618 | 16.38 | 4.47 | 5.21 | 4.50 | 3.42 | 8.30 |
| 41 | SRS-000619 | 11.33 | 5.10 | 2.04 | 14.32 | 9.24 | 7.83 |
| 42 | SRS-000620 | 29.95 | 33.73 | 12.94 | 5.09 | 4.74 | 8.28 |
| 43 | SRS-000621 | 43.86 | 40.21 | 27.13 | 1.38 | 1.68 | 0.57 |
| 44 | SRS-000622 | 35.67 | 27.25 | 33.88 | 11.97 | 5.24 | 10.31 |
| 45 | SRS-000623 | 17.60 | 9.54 | −37.59 | 5.63 | 5.31 | 21.04 |
| 46 | SRS-000624 | 30.34 | 35.02 | 29.58 | 6.17 | 14.91 | 9.90 |
| 47 | SRS-000625 | 3.16 | −11.17 | −6.26 | 11.59 | 12.77 | 5.57 |
| 48 | SRS-000626 | 17.17 | 14.48 | 3.19 | 19.46 | 3.31 | 5.75 |
| 49 | SRS-000627 | 48.55 | 36.56 | 33.95 | 9.27 | 9.61 | 8.65 |
| 50 | SRS-000628 | 59.91 | 61.20 | 61.90 | 8.34 | 4.64 | 2.02 |

TABLE 4
Results of Positive Control for In vitro efficacy of siRNAs targeting FXI

| | positive | Average Inhibition % | | | SD | | |
|---|---|---|---|---|---|---|---|
| No. | control | 100 nM | 30 nM | 10 nM | 100 nM | 30 nM | 10 nM |
| plate 1 | SRS-000589 | 72.83 | 70.28 | 59.21 | 10.54 | 1.80 | 7.18 |
| plate 2 | SRS-000589 | 79.92 | 74.43 | 64.77 | 3.25 | 0.21 | 1.80 |
| plate 3 | SRS-000589 | 69.89 | 65.92 | 61.77 | 9.42 | 11.85 | 5.94 |
| plate 4 | SRS-000589 | 61.37 | 53.25 | 43.85 | 13.88 | 7.18 | 4.83 |
| plate 5 | SRS-000589 | 67.72 | 67.47 | 56.93 | 7.60 | 1.23 | 4.41 |
| plate 6 | SRS-000589 | 69.45 | 62.87 | 47.09 | 9.12 | 3.94 | 9.87 |

TABLE 5
Results of Free Uptake Dose Response
in Primary Human Hepatocytes

| | | | Maximum % |
|---|---|---|---|
| | | Maximum % | Inhibition |
| Duplex ID | IC50 (nM) | Inhibition | SD |
| SRS-001724 | 1.5 | 62.0 | 10.6 |
| SRS-001733 | 2.3 | 66.7 | 2.0 |
| SRS-001725 | 1.5 | 68.3 | 1.5 |
| SRS-001734 | 0.4 | 67.2 | 4.7 |
| SRS-001726 | 1.0 | 59.3 | 6.3 |
| SRS-001735 | 1.3 | 62.3 | 4.1 |
| SRS-001727 | 0.3 | 44.5 | 12.2 |
| SRS-001736 | 0.2 | 47.5 | 8.7 |
| SRS-001728 | 1.8 | 75.4 | 1.7 |
| SRS-001737 | 0.4 | 66.3 | 1.6 |
| SRS-000007 | 0.2 | 23.8 | 1.2 |
| SRS-001738 | 2.3 | 56.6 | 4.0 |
| SRS-001729 | 7.7 | 52.3 | 2.2 |
| SRS-001739 | 2.4 | 57.0 | 4.2 |
| SRS-001730 | NA | 41.7 | 6.3 |
| SRS-001740 | 51.7 | 65.1 | 1.9 |
| SRS-001731 | 1.1 | 75.3 | 1.7 |
| SRS-001741 | 1.7 | 75.4 | 1.0 |
| SRS-001732 | 0.2 | 31.7 | 3.9 |
| SRS-001742 | 0.6 | 55.0 | 2.9 |

TABLE 6
Results of Free Uptake Dose Response in
Three Primary Human Hepatocyte Donors

| | | | | Maximum % |
|---|---|---|---|---|
| | | | Maximum % | Inhibition |
| Duplex ID | PHH Lot ID | IC50 (nM) | Inhibition | SD |
| SRS-000007 | LGI | 7.7 | 79% | 3.9 |
| | RAS | 4.2 | 76% | 2.6 |
| | XVF | 3.6 | 78% | 3.6 |

TABLE 7
Sequence Modifications Evaluated in Cynomolgus Monkeys

| | Target | SEQ | | SEQ | | SEQ | | SEQ | |
|---|---|---|---|---|---|---|---|---|---|
| | Posi- | ID | Guide/Antisense Structure | ID | Guide/ Antisense Base | ID | Passenger/Sense Structure | ID | Passenger/Sense Base |
| Duplex ID | tion | NO. | Code (5′-3′) | NO. | Sequence (5′-3′) | NO. | Code (5′-3′) | NO. | Sequence (5′-3′) |
| SRS-000007 | 433 | 61 | asUfsasaugucuuUfgUfuGfcaagcsgsc | 11 | AUAAAUGUCUUUGUUGCAAGCGC | 161 | gscsuugcAfaCfaAfagacauuuau | 111 | GCUUGCAACAAAGACAUUUAU |
| SRS-000236 | 433 | 211 | asUfsaaaugucuuUfgUfuGfcsaagcsgsc | 11 | AUAAAUGUCUUUGUUGCAAGCGC | 161 | gscsuugcAfaCfaAfagacauuuau | 111 | GCUUGCAACAAAGACAUUUAU |
| SRS-000255 | 433 | 212 | asUfsaaaugucuuUfgUfsuGfcaagcsgsc | 11 | AUAAAUGUCUUUGUUGCAAGCGC | 161 | gscsuugcAfaCfaAfagacauuuau | 111 | GCUUGCAACAAAGACAUUUAU |
| SRS-000257 | 433 | 213 | asUfsaaaugucuuUfgUfuGfcaasgcsgsc | 11 | AUAAAUGUCUUUGUUGCAAGCGC | 161 | gscsuugcAfaCfaAfagacauuuau | 111 | GCUUGCAACAAAGACAUUUAU |
| SRS-000258 | 433 | 61 | asUfsaaaugucuuUfgUfuGfcaagcsgsc | 11 | AUAAAUGUCUUUGUUGCAAGCGC | 214 | gscsuugcAfaCfaAfagacauuuau | 111 | GCUUGCAACAAAGACAUUUAU |
| | | | | | | | (invdT)(invdT) | | |

Note:
the target position is relative to human transcript NM_000128. 4; the corresponding sequence of a certain SEQ ID NO. is located on its right column of the same row. “A” refers to adenosine-3′-phosphate, “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u″ refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; ″t” refers to 2′-O-methyl-5-methyluridine-3′-phosphate; “Tf′ refers to 2′-fluoro-5-methyluridine-3′-phosphate; “dT” refers to 2′-deoxythymidine-3′-phosphate; “s” refers to 3′-phosphorothioate; (invdT) refers to 3′ inverted thymidine; Note that, in some instances, the passenger/sense structure code or sense strand sequence of SRS-000007, SRS-000236, SRS-000255, or SRS-000257 is further coupled to X2-GalNAc moiety via formula (V′),(V″), (V″″), (V″) at the 3′ end of the sense strand; Note that, in some instances, the passenger/sense structure code or sense strand sequence of SRS-000258 is further coupled to X2-GalNAc moiety via formula (V″) at the S′ end of the sense strand

TABLE 8
Additional Sequences Evaluated in Cynomolgus Monkeys

| | Target | SEQ | | SEQ | | SEQ | | SEQ | |
|---|---|---|---|---|---|---|---|---|---|
| | Posi- | ID | Guide/Antisense Structure | ID | Guide/Antisense Base | ID | Passenger/Sense Structure | ID | Passenger/Sense Base |
| Duplex ID | tion | NO. | Code (5′-3′) | NO. | Sequence (5′-3′) | NO. | Code (5′-3′) | NO. | Sequence (5′-3′) |
| SRS-001736 | 429 | 204 | asUfsgucuUfuguuGfcAfaGfcgcuusasu | 9 | AUGUCUUUGUUGCAAGCGCUUAU | 159 | asasgcgcUfuGfcAfacaaagacau | 109 | AAGCGCUUGCAACAAAGACAU |
| SRS-001737 | 430 | 205 | asAfsugucUfuuguUfgCfaAfgegcususa | 10 | AAUGUCUUUGUUGCAAGCGCUUA | 160 | asgscgcuUfgCfaAfcasagacauu | 110 | AGCGCUUGCAACAAAGACAUU |
| SRS-001738 | 433 | 206 | asUfsaaauGfucuuUfgUfuGfcaagcsgsc | 11 | AUAAAUGUCUUUGUUGCAAGCGC | 161 | gscsuugcAfaCfaAfagacauuuau | 111 | GCUUGCAACAAAGACAUUUAU |
| SRS-001739 | 465 | 207 | usUfsauagUfuuauGfcCfcUfucaugsusc | 13 | UUAUAGUUUAUGCCCUUCAUGUC | 163 | csasugaaGfgGfcAfuaaacuauaa | 113 | CAUGAAGGGCAUAAACUAUAA |
| SRS-001741 | 1084 | 209 | asUfsagguAfaaaaAfcUfgGfcagcgsgsa | 35 | AUAGGUAAAAAACUGGCAGCGGA | 185 | csgscugcCfaGfuUfuuuuaccuau | 135 | CGCUGCCAGUUUUUUACCUAU |

Note:
the target position is relative to human transcript NM_000128.4; the corresponding sequence of a certain SEQ ID NO. is located on its right column of the same row. “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “G” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t″ refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate; Note that, in some instances, the passenger/sense strand sequence is further coupled to X2-GalNAc moiety via formula (V′), (V″), (V″″″), (V″″″) at the 3′ end of the sense strand

TABLE 9
Additional siRNA sequences

| | Target | SEQ | | SEQ | | SEQ | | SEQ | |
|---|---|---|---|---|---|---|---|---|---|
| | Posi- | ID | Guide/Antisense Structure | ID | Guide/Antisense Base | ID | Passenger/sense Structure | ID | Passenger/sense Base |
| Duplex ID | tion | NO. | Code (5′-3′) | NO. | Sequence (5′-3′) | NO. | Code (5′-3′) | NO. | Sequence (5′-3′) |
| SRS-000007 | 433 | 61 | asUfsaaaugucuuUfgUfuGfcaagcsgsc | 11 | AUAAAUGUCUUUGUUGCAAG | 161 | gscsuugcAfaCfaAfagacauuuau | 111 | GCUUGCAACAAAGACAUUUAU |
| | | | | | CGC | | | | |
| SRS-002331 | 433 | 217 | isUfsaaaugucuuUfgUfuGfcaagcsgsc | 215 | IUAAAUGUCUUUGUUGCAAG | 161 | gscsuugcAfaCfaAfagacauuuau | 111 | GCUUGCAACAAAGACAUUUAU |
| | | | | | CGC | | | | |
| SRS-002376 | 433 | 218 | usUfsaaauGfucuuUfgUfuGfcaagcsgs | 216 | UUAAAUGUCUUUGUUGCAAG | 221 | gscsuugcAfaCfaAfagacauuuaa | 220 | GCUUGCAACAAAGACAUUUAA |
| | | | c | | CGC | | | | |
| SRS-002377 | 433 | 219 | a4sUfsaaauGfucuuUfgUfuGfcaagcsg | 11 | AUAAAUGUCUUUGUUGCAAG | 161 | gscsuugcAfaCfaAfagacauuuau | 111 | GCUUGCAACAAAGACAUUUAU |
| | | | sc | | CGC | | | | |
| SRS-000233 | 433 | 223 | a(Rps)Uf(Rps)aaaugucuuUfgUfuGfc | 11 | AUAAAUGUCUUUGUUGCAAG | 161 | gscsuugcAfaCfaAfagacauuuau | 111 | GCUUGCAACAAAGACAUUUAU |
| | | | aagc(Sps)gsc | | CGC | | | | |
| SRS-000234 | 433 | 224 | a(Rps)Uf(Rps)a(Rps)aaugucuuUfgU | 11 | AUAAAUGUCUUUGUUGCAAG | 161 | gscsuugcAfaCfaAfagacauuuau | 111 | GCUUGCAACAAAGACAUUUAU |
| | | | fuGfcaagc(Sps)gsc | | CGC | | | | |
| SRS-000235 | 433 | 225 | asUfsaaasugucuuUfgUfuGfcaagcsgs | 11 | AUAAAUGUCUUUGUUGCAAG | 161 | gscsuugcAfaCfaAfagacauuuau | 111 | GCUUGCAACAAAGACAUUUAU |
| | | | c | | CGC | | | | |
| SRS-000237 | 433 | 226 | asUfsaaasugucuuUfgUfuGfcsaagcsg | 11 | AUAAAUGUCUUUGUUGCAAG | 161 | gscsuugcAfaCfaAfagacauuuau | 111 | GCUUGCAACAAAGACAUUUAU |
| | | | sc | | CGC | | | | |
| SRS-000256 | 433 | 227 | asUfsaaasugucuuUfgUfsuGfcaagcsg | 11 | AUAAAUGUCUUUGUUGCAAG | 161 | gscsuugcAfaCfaAfagacauuuau | 111 | GCUUGCAACAAAGACAUUUAU |
| | | | sc | | CGC | | | | |

Note:
the target position is relative to human transcript NM_000128.4; the corresponding sequence of a certain SEQ ID NO. is located on its right column of the same row. “A” refers to adenosine-3′-phosphate; “a” refers to 2′-O-methyladenosine-3′-phosphate; “Af” refers to 2′ -fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′ phosphate; “Gf′ refers to 2′-fluoroguanosine-3′-phosphate; “dG″ refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “i” refers to 2′-O-methylinosine-3′-phosphate, “a4” refers to 2′-O-methyl-8-bromo-adenosine-3′-phopshate; Note that the passenger/sense structure code or sense strand sequence is further coupled to X2-GalNAc moiety via formula (V′) at the 3′ end of the sense strand

TABLE 10
In vitro results for two siRNAs in two primary human hepatocyte donors
Free Uptake in PHH

| | | | Max % | Max % |
|---|---|---|---|---|
| ID | Donor | Rel IC50 (nM) | Inhibition | Inhibition SD |
| SRS-002331 | BXU | 5.1 | 94.6 | 0.7 |
| SRS-000007 | | 7.8 | 95.3 | 0.9 |
| SRS-002331 | JMJ | 8.4 | 89.2 | 0.5 |
| SRS-000007 | | 11.3 | 92.9 | 1.2 |

What is claimed is:

1. A polynucleic acid molecule for modulating expression of coagulation factor XI (FXI) gene, wherein the polynucleic acid molecule comprises a nucleic acid sequence that is at least 80%, at least 85%, at least 90% identical to a nucleic acid sequence in Tables 1-2 and Tables 7-9.

2. The polynucleic acid molecule of claim 1, wherein the polynucleic acid molecule is a double-stranded nucleic acid molecule comprising a sense strand (passenger strand) and an antisense strand (guide strand).

3. The polynucleic acid molecule of claim 2, wherein the sense strand comprises a nucleic acid sequence that is at least 80%, at least 85%, at least 90%, at least 95% identical to a nucleic acid sequence selected from SEQ ID NOs: 101-150 and 220.

4. The polynucleic acid molecule of any one of claims 2-3, wherein the antisense strand comprises a nucleic acid sequence that is at least 80%, at least 85%, at least 90%, at least 95% identical to a nucleic acid sequence selected from SEQ ID NOs: 1-50 and 215-216.

5. The polynucleic acid molecule of any one of claims 2-4, wherein the sense strand comprises a nucleic acid sequence comprising at least 14, 15, 16, 17, 18, 19, or 20 consecutive sequences of a nucleic acid sequence selected from SEQ ID NOs: 101-150 and 220 with no more than 1, 2, 3, or 4 mismatches.

6. The polynucleic acid molecule of any one of claims 2-5, wherein the antisense strand comprises a nucleic acid sequence comprising at least 14, 15, 16, 17, 18, 19, 20, 21, or 22 consecutive sequences of a nucleic acid sequence selected from SEQ ID NOs:1-50 and 215-216 with no more than 1, 2, 3, or 4 mismatches.

7. The polynucleic acid molecule of any one of claims 2-6, wherein the sense strand comprises a nucleic acid sequence of SEQ ID NOs: 101-150 and 220 and the antisense strand comprises a nucleic acid sequence of SEQ ID NOs: 1-50 and 215-216.

8. The polynucleic acid molecule of any one of claims 2-7, wherein the sense strand comprises a nucleic acid sequence that is at least 90%, at least 95% identical to a nucleic acid sequence selected from SEQ ID NOs: 104, 106-107, 109-111, 113-114, 135, 150, and 220.

9. The polynucleic acid molecule of any one of claims 2-8, wherein the antisense strand comprises a nucleic acid sequence that is at least 90%, at least 95% identical to a nucleic acid sequence selected from SEQ ID NOs: 4, 6-7, 9-11, 13-14, 35, 50, and 215-216.

10. The polynucleic acid molecule of any one of claims 2-9, wherein the sense strand comprises a nucleic acid sequence selected from SEQ ID NOs: 104, 106-107, 109-111, 113-114, 135, 150, and 220 and the antisense strand comprises a nucleic acid sequence selected from a nucleic acid sequence of SEQ ID NOs: 4, 6-7, 9-11, 13-14, 35, 50, and 215-216.

11. The polynucleic acid molecule of any one of claims 2-10, wherein the antisense strand comprises 5′-nNfnnnNfnNfNfnnnnNfnNfnnnnnnn-3′, 5′-nNfnnnNfnnnnnnnNfnNfnnnnnnn-3′, 5′-nNfnnnnNfnnnnNfnNfnnnnnnnnn-3′, 5′-nNfnnnnNfnnnnNfnNfnNfnnnnnnn-3′, or 5′-nNfnnnnnnnnnNfnNfnNfnnnnnnn-3′, wherein “Nf” stands for a 2′-fluoro modified nucleotide, and wherein “n” stands for a 2′-O-methyl modified nucleotide.

12. The polynucleic acid molecule of any one of claims 2-11, wherein the sense strand comprises 5′-nnnnnnNfnNfnNfnnnnnnnnnn-3′, 5′-nnnnnnNfnNfNfNfnnnnnnnnnn-3′, 5′-nnnnnnnnNfNfNfnnnnnnnnnn-3′, or 5′-nnnnnnNfnNfnNfnnnnnnnnnn-invdN-invdN-3′ wherein “Nf” stands for a 2′-fluor, modified nucleotide, and wherein “n” stands for a 2′-O-methyl modified nucleotide, and wherein “invdN” stands for an inverted deoxy-nucleotide.

13. The polynucleic acid molecule of any one of claims 2-12, wherein the sense strand comprises 5′-nnnnnnNfnNfnNfnnnnnnnnnn-3′, wherein the antisense strand comprises 5′-nNfnnnnnnnnnNfnNfnNfnnnnnnn-3′, wherein “Nf” stands for a 2′-fluoro modified nucleotide, and wherein “n” stands for a 2′-O-methyl modified nucleotide.

14. The polynucleic acid molecule of any one of claims 2-12, wherein the sense strand comprises 5′-nnnnnnNfnNfnNfnnnnnnnnnn-3′, wherein the antisense strand comprises 5′-nNfnnnnNfnnnnNfnNfnNfnnnnnnn-3′, wherein “Nf” stands for a 2′-fluoro modified nucleotide, and wherein “n” stands for a 2′-O-methyl modified nucleotide.

15. The polynucleic acid molecule of any one of claims 1-14, wherein the polynucleic acid molecule comprises a modified internucleotide linkage, optionally wherein the modified internucleotide linkage is a phosphorothioate internucleotide linkage.

16. The polynucleic acid molecule of claim 15, wherein the modified internucleotide linkage comprises a stereochemically enriched phosphorothioate internucleotide linkage.

17. The polynucleic acid molecule of any one of claims 15-16, wherein the modified internucleotide linkage is an SP chiral internucleotide phosphorothioate linkage.

18. The polynucleic acid molecule of any one of claims 15-17, wherein the polynucleic acid comprises a plurality of modified internucleotide linkages, and at least 1, 2, 3, or 4 of the plurality of modified internucleotide linkages are stereochemically enriched phosphorothioate internucleotide linkages.

19. The polynucleic acid molecule of claim 18, wherein the stereochemically enriched phosphorothioate internucleotide linkages comprise both R- and S-isomers.

20. The polynucleic acid molecule of one of claims 18-19, wherein the stereochemically enriched phosphorothioate is disposed between two consecutive nucleosides that are two of six 5′ or 3′-terminal nucleosides of the sense strand or the antisense strand.

21. The polynucleic acid molecule of any one of claims 1-20, wherein the polynucleic acid molecule comprises a hypoxanthine nucleobase-containing nucleoside substitution.

22. The polynucleic acid molecule of claim 21, wherein the hypoxanthine nucleobase-containing nucleoside substitution is an inosine substitution, optionally wherein the inosine substitution comprises 2′-O-methylinosine-3′-phosphate.

23. The polynucleic acid molecule of claim 22, wherein the inosine substitution is within a seed region of the antisense strand.

24. The polynucleic acid molecule of claim 22, wherein the inosine substitution is within 7 nucleotides from the 5′ end of the antisense strand, optionally wherein the inosine substitution is in the first nucleotide from the 5′ end of the antisense strand.

25. The polynucleic acid molecule of any one of claims 1-24, wherein the first nucleotide from the 5′ end of the antisense strand is substituted by a uridine or an adenosine, optionally wherein the uridine comprises 2′-O-methy luridine-3′-phosphate, or optionally wherein the adenosine comprises 2′-O-methyl-8-bromo-adenosine-3′-phopshate.

26. The polynucleic acid molecule of any one of claims 1-25, wherein the sense strand comprises a nucleic acid sequence that is at least 80%, at least 85%, at least 90%, at least 95% identical to a nucleic acid sequence selected from SEQ ID NOs: 151-200, 214, and 221.

27. The polynucleic acid molecule of any one of claims 1-26, wherein the antisense strand comprises a nucleic acid sequence that is at least 80%, at least 85%, at least 90%, at least 95% identical to a nucleic acid sequence selected from SEQ ID NOs: 51-100, 201-213, and 217-219.

28. The polynucleic acid molecule of any one of claims 1-27, wherein the sense strand comprises a nucleic acid sequence selected from a nucleic acid sequence of SEQ ID NOs: 151-200, 214, and 221 and the antisense strand comprises a nucleic acid sequence selected from a nucleic acid sequence of SEQ ID NOs: 51-100, 201-213, and 217-219.

29. A polynucleic acid molecule for modulating expression of coagulation factor XI (FXI) gene, wherein the polynucleic acid molecule comprises:
(a) an antisense strand comprising a nucleotide sequence of SEQ ID NO: 4, 6-7, 9-11, 13-14, 35, 50, and 215-216 and a sense strand comprising a nucleotide sequence of SEQ ID NO: 104, 106-107, 109-111, 113-114, 135, 150, and 220; or
(b) an antisense strand comprising a nucleotide sequence of SEQ ID NO: 54, 56-57, 59-61, 63-64, 85, 100, 201-213, and 217-219 and a sense strand comprising a nucleotide sequence of SEQ ID NO: 154, 156-157, 159-161, 163-164, 185, 200, 214, and 221.

30. The polynucleic acid molecule of claim 29, wherein the poly nucleic acid molecule comprise:
(a) an antisense strand comprising a nucleotide sequence of AUAAAUGUCUUUGUUGCAAGCGC (SEQ ID NO:11) and a sense strand comprising a nucleotide sequence of GCUUGCAACAAAGACAUUUAU(SEQ ID NO: 111),
(b) an antisense strand comprising a nucleotide sequence of AUGUCUUUGUUGCAAGCGCUUAU (SEQ ID NO: 9) and a sense strand comprising a nucleotide sequence of AAGCGCUUGCAACAAAGACAU (SEQ ID NO: 109);
(c) an antisense strand comprising a nucleotide sequence of AAUGUCUUUGUUGCAAGCGCUUA (SEQ ID NO: 10) and a sense strand comprising a nucleotide sequence of AGCGCUUGCAACAAAGACAUU (SEQ ID NO: 110);
(d) an antisense strand comprising a nucleotide sequence of UUAUAGUUUAUGCCCUUCAUGUC (SEQ ID NO: 13) and a sense strand comprising a nucleotide sequence of CAUGAAGGGCAUAAACUAUAA (SEQ ID NO: 113);
(e) an antisense strand comprising a nucleotide sequence of AUAGGUAAAAAACUGGCAGCGGA (SEQ ID NO: 35) and a sense strand comprising a nucleotide sequence of CGCUGCCAGUUUUUUACCUAU (SEQ ID NO: 135)
(f) an antisense strand comprising a nucleotide sequence of IUAAAUGUCUUUGUUGCAAGCGC (SEQ ID NO: 215) and a sense strand comprising a nucleotide sequence of GCUUGCAACAAAGACAUUUAU (SEQ ID NO: 111); or
(g) an antisense strand comprising a nucleotide sequence of UUAAAUGUCUUUGUUGCAAGCGC (SEQ ID NO: 216) and a sense strand comprising a nucleotide sequence of GCUUGCAACAAAGACAUUUAA (SEQ ID NO: 220).

31. A polynucleic acid molecule for modulating expression of coagulation factor XI (FXI) gene, wherein the polynucleic acid molecule comprises:
(a) an antisense strand comprising a nucleotide sequence of asUfsaaaugucuuUfgUfuGfcaagcsgsc (SEQ ID NO: 61) and a sense strand comprising a nucleotide sequence of gscsuugcAfaCfaAfagacauuuau (SEQ ID NO: 161);
(b) an antisense strand comprising a nucleotide sequence of asUfsaaaugucuuUfgUfuGfcsaagcsgsc (SEQ ID NO: 211) and a sense strand comprising a nucleotide sequence of gscsuugcAfaCfaAfagacauuuau (SEQ ID NO: 161);
(c) an antisense strand comprising a nucleotide sequence of asUfsaaaugucuuUfgUfsuGfcaagcsgsc (SEQ ID NO: 212) and a sense strand comprising a nucleotide sequence of gscsuugcAfaCfaAfagacauuuau (SEQ ID NO: 161);
(d) an antisense strand comprising a nucleotide sequence of asUfsaaaugucuuUfgUfuGfcaasgcsgsc (SEQ ID NO: 213) and a sense strand comprising a nucleotide sequence of gscsuugcAfaCfaAfagacauuuau (SEQ ID NO: 161);
(e) an antisense strand comprising a nucleotide sequence of asUfsaaaugucuuUfgUfuGfcaagcsgsc (SEQ ID NO: 61) and a sense strand comprising a nucleotide sequence of gscsuugcAfaCfaAfagacauuuau(invdT)(invdT) (SEQ ID NO: 214);
(f) an antisense strand comprising a nucleotide sequence of asUfsgucuUfuguuGfcAfaGfcgcuusasu (SEQ ID NO: 204) and a sense strand comprising a nucleotide sequence of asasgcgcUfuGfcAfacaaagacau (SEQ ID NO: 159);
(g) an antisense strand comprising a nucleotide sequence of asAfsugucUfuuguUfgCfaAfgcgcususa (SEQ ID NO: 205) and a sense strand comprising a nucleotide sequence of asgscgcuUfgCfaAfcaaagacauu (SEQ ID NO: 160);
(h) an antisense strand comprising a nucleotide sequence of asUfsaaauGfucuuUfgUfuGfcaagcsgsc (SEQ ID NO: 206) and a sense strand comprising a nucleotide sequence of gscsuugcAfaCfaAfagacauuuau (SEQ ID NO: 161);
(i) an antisense strand comprising a nucleotide sequence of usUfsauagUfuuauGfcCfcUfucaugsusc (SEQ ID NO: 207) and a sense strand comprising a nucleotide sequence of csasugaaGfgGfcAfuaaacuauaa (SEQ ID NO: 163);
(j) an antisense strand comprising a nucleotide sequence of asUfsagguAfaaaaAfcUfgGfcagcgsgsa (SEQ ID NO: 209) and a sense strand comprising a nucleotide sequence of csgscugcCfaGfuUfuuuuaccuau (SEQ ID NO: 185);
(k) an antisense strand comprising a nucleotide sequence of isUfsaaaugucuuUfgUfuGfcaagcsgsc (SEQ ID NO: 217) and a sense strand comprising a nucleotide sequence of gscsuugcAfaCfaAfagacauuuau (SEQ ID NO: 161);
(l) an antisense strand comprising a nucleotide sequence of usUfsaaauGfucuuUfgUfuGfcaagcsgsc (SEQ ID NO: 218) and a sense strand comprising a nucleotide sequence of gscsuugcAfaCfaAfagacauuuaa (SEQ ID NO: 222); or
(m) an antisense strand comprising a nucleotide sequence of a4sUfsaaauGfucuuUfgUfuGfcaagcsgsc (SEQ ID NO: 219) and a sense strand comprising a nucleotide sequence of gscsuugcAfaCfaAfagacauuuau (SEQ ID NO: 161);
wherein “A” refers to adenosine-3′-phosphate; “a” refers to 2′-O-methyladenosine-3′-phosphate; “Af” refers to 2′-fluoroadenosine-3′-phosphate; “dA” refers to 2′-deoxyadenosine-3-phosphate; “C” refers to cytidine-3′-phosphate; “c” refers to 2′-O-methylcytidine-3′-phosphate; “Cf” refers to 2′-fluorocytidine-3′-phosphate; “dC” refers to 2′-deoxycytidine-3′-phosphate; “G” refers to guanosine-3′-phosphate; “g” refers to 2′-O-methylguanosine-3′-phosphate; “Gf” refers to 2′-fluoroguanosine-3′-phosphate; “dG” refers to 2′-deoxyguanosine-3′-phosphate; “U” refers to uridine-3′-phosphate; “u” refers to 2′-O-methyluridine-3′-phosphate; “Uf” refers to 2′-fluorouridine-3′-phosphate; “dU” refers to 2′-deoxyuridine-3′-phosphate; “T” refers to 5-methyluridine-3′-phosphate; “t” refers to 2′-O-methyl-5-methyluridine-3′-phosphate; “Tf” refers to 2′-fluoro-5-methyluridine-3′-phosphate; “dT” refers to 2′-deoxythymidine-3′-phosphate; “s” refers to 3′-phosphorothioate, “invdT” refers to 3′ inverted thymidine, “i” refers to 2′-O-methylinosine-3′-phosphate, and “a4” refers to 2′-O-methyl-8-bromo-adenosine-3′-phopshate.

32. A polynucleic acid molecule conjugate for modulating expression of coagulation factor XI (FXI) gene, wherein the polynucleic acid molecule conjugate comprises a polynucleic acid molecule of any one of claims 1-31 and an asialoglycoprotein receptor targeting moiety.

33. The polynucleic acid molecule conjugate of claim 32, wherein the polynucleic acid molecule and the asialoglycoprotein receptor targeting moiety is coupled via a linker.

34. The polynucleic acid molecule conjugate of claim 33, wherein the linker comprises formula (IV) below, 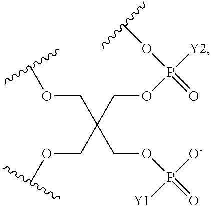
wherein at least one of Y1 and Y2 is a nucleotide in the polynucleic acid molecule.

35. The polynucleic acid molecule conjugate of claim 34, wherein the Y1 is the last nucleotide on the 3′-terminus of the sense strand of the polynucleic acid molecule, or wherein the Y2 is the first nucleotide on the 5′-terminus of the sense strand of the polynucleic acid molecule.

36. The polynucleic acid molecule conjugate of claim 34, wherein the Y1 and Y2 are two consecutive nucleotides in the polynucleic acid molecule.

37. The polynucleic acid molecule conjugate of any one of claims 32-36, wherein the asialoglycoprotein receptor targeting moiety comprises N-Acetylgalactosamine (GalNAc) or galactose.

38. The polynucleic acid molecule conjugate of any one of claims 33-36, wherein the linker and the asialoglycoprotein receptor targeting moiety with the last nucleotide on the 3′-terminus of the sense strand of the polynucleic acid molecule are shown in: 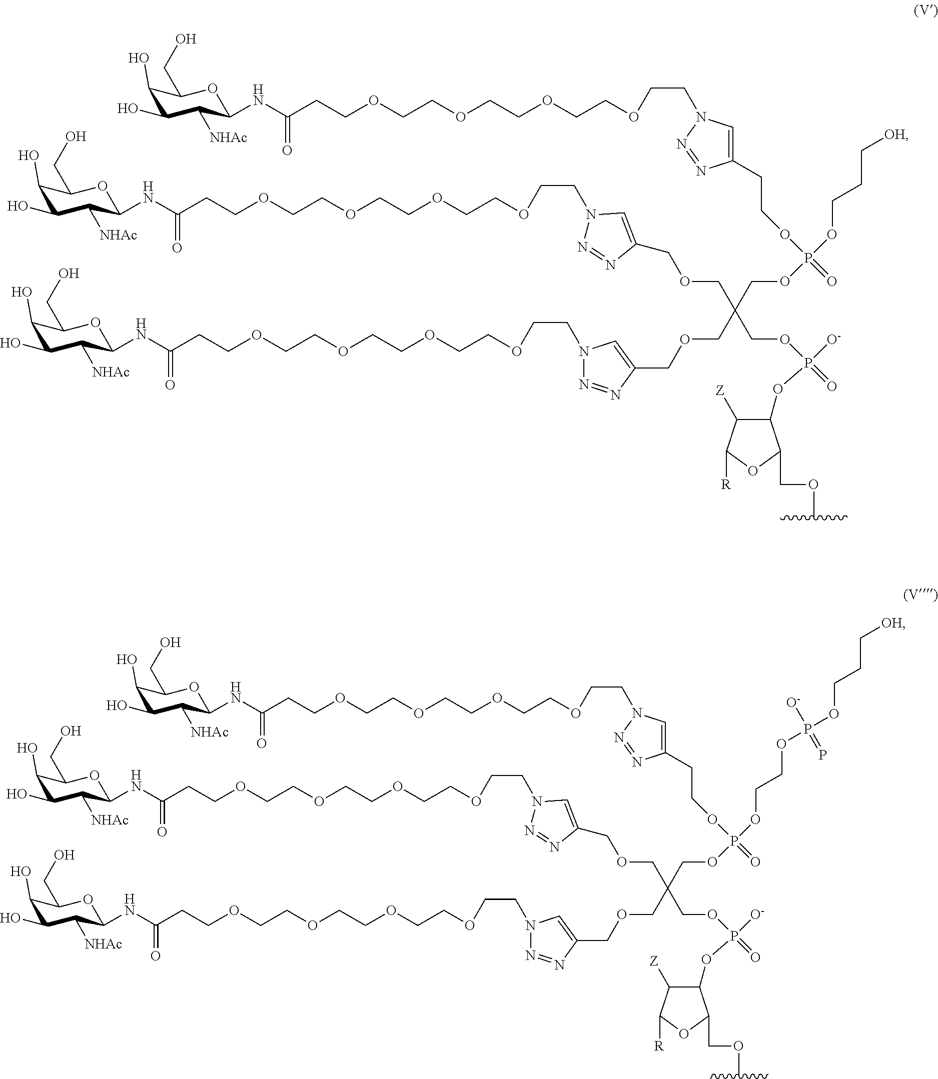 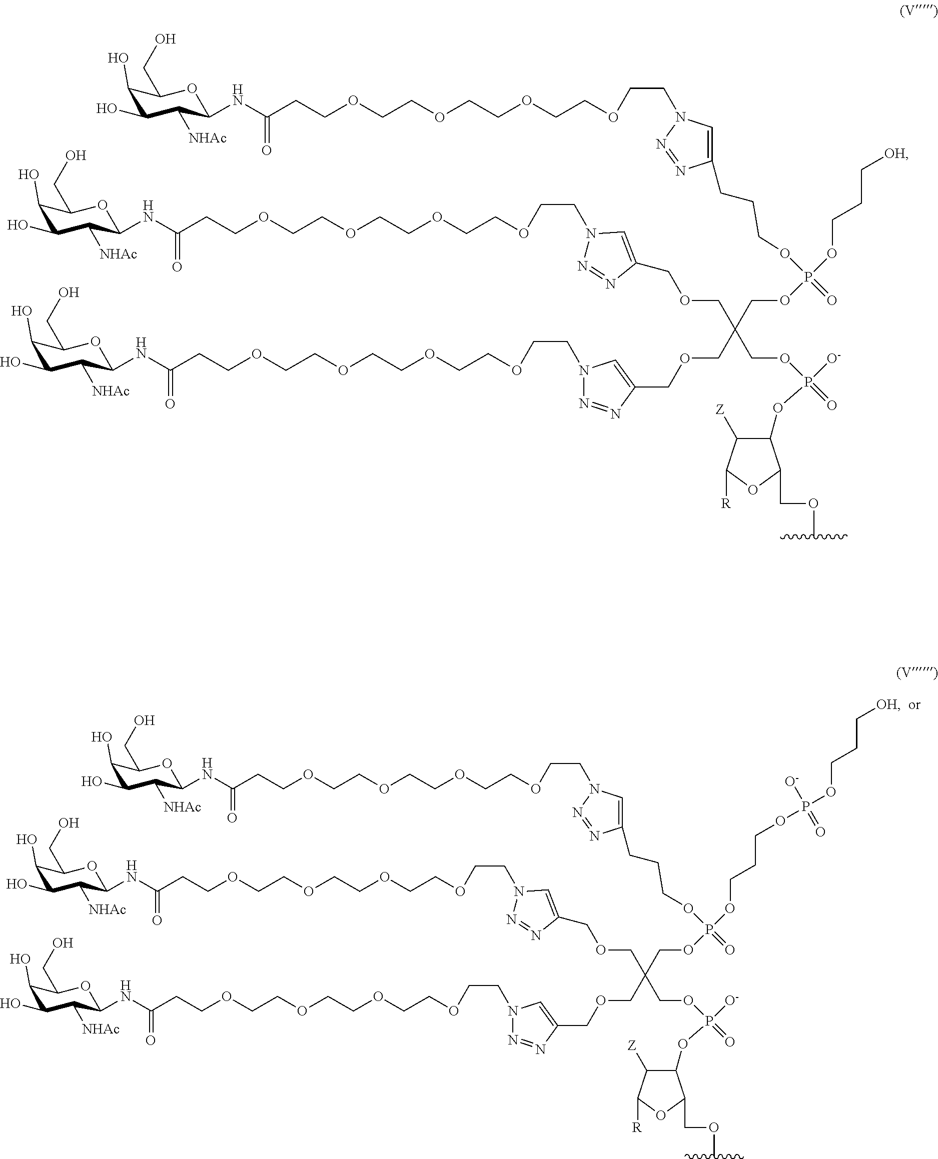
wherein Z in formula (V′), (V″″), (V′″″), or (V″″″) is —H, —OH, —O-Methyl, —F, or —O-methoxyethyl, and R in formula (V′) is adenine, uracil, guanine, cytosine, thymine, abasic, or others.

39. A pharmaceutical composition comprising a polynucleic acid molecule of any one of claims 1-31 or a polynucleic acid molecule conjugate of any one of claims 32-38, and a pharmaceutically acceptable excipient.

40. A method of modulating mRNA expression of coagulation factor XI (FXI) gene in a subject, comprising: administering to the subject a polynucleic acid molecule of any one of claims 1-31 or a polynucleic acid molecule conjugate of any one of claims 32-38, or a pharmaceutical composition of claim 39, thereby modulating the mRNA expression of FXI gene in the subject.

41. A method of modulating FXI or FXIa protein levels or FXI or FXIa activity in a subject in need thereof, comprising: administering to the subject a polynucleic acid molecule of any one of claims 1-31 or a polynucleic acid molecule conjugate of any one of claims 32-38, or a pharmaceutical composition of claim 39, thereby modulating the FXI or FXIa protein levels or FXI or FXIa activity in the subject.

42. The method of claim 41, wherein the subject in need thereof suffers from thrombosis or a symptom thereof.


SEQUENCE LISTING (229 sequences)

SEQ ID NO: 1 - RNA, 23 nt, synthetic construct
  source 1..23: mol_type = other RNA
agtcacacattcaccagaaactg

SEQ ID NO: 2 - RNA, 23 nt, synthetic construct
  source 1..23: mol_type = other RNA
aatgtcccctccttcaaagcagg

SEQ ID NO: 3 - RNA, 23 nt, synthetic construct
  source 1..23: mol_type = other RNA
attcactcttggcagtgtttctg

SEQ ID NO: 4 - RNA, 23 nt, synthetic construct
  source 1..23: mol_type = other RNA
tgaaagaatacccagaaatcgct

SEQ ID NO: 5 - RNA, 23 nt, synthetic construct
  source 1..23: mol_type = other RNA
ttgcttgaaagaatacccagaaa

SEQ ID NO: 6 - RNA, 23 nt, synthetic construct
  source 1..23: mol_type = other RNA
attgcttgaaagaatacccagaa

SEQ ID NO: 7 - RNA, 23 nt, synthetic construct
  source 1..23: mol_type = other RNA
tattgcttgaaagaatacccaga

SEQ ID NO: 8 - RNA, 23 nt, synthetic construct
  source 1..23: mol_type = other RNA
tatttggtgtgagcattgcttga

SEQ ID NO: 9 - RNA, 23 nt, synthetic construct
  source 1..23: mol_type = other RNA
atgtctttgttgcaagcgcttat

SEQ ID NO: 10 - RNA, 23 nt, synthetic construct
  source 1..23: mol_type = other RNA
aatgtctttgttgcaagcgctta

SEQ ID NO: 11 - RNA, 23 nt, synthetic construct
  source 1..23: mol_type = other RNA
ataaatgtctttgttgcaagcgc

SEQ ID NO: 12 - RNA, 23 nt, synthetic construct
  source 1..23: mol_type = other RNA
acataaatgtctttgttgcaagc

SEQ ID NO: 13 - RNA, 23 nt, synthetic construct
  source 1..23: mol_type = other RNA
ttatagtttatgcccttcatgtc

SEQ ID NO: 14 - RNA, 23 nt, synthetic construct
  source 1..23: mol_type = other RNA
tttcttggcattcttgagcactc

SEQ ID NO: 15 - RNA, 23 nt, synthetic construct
  source 1..23: mol_type = other RNA
acaaatgttacgatgctccaggc

SEQ ID NO: 16 - RNA, 23 nt, synthetic construct
  source 1..23: mol_type = other RNA
tagtagacaaatgttacgatgct

SEQ ID NO: 17 - RNA, 23 nt, synthetic construct
  source 1..23: mol_type = other RNA
tttcagtagacaaatgttacgat

SEQ ID NO: 18 - RNA, 23 nt, synthetic construct
  source 1..23: mol_type = other RNA
tatttcagtgaaaatccagacac

SEQ ID NO: 19 - RNA, 23 nt, synthetic construct
  source 1..23: mol_type = other RNA
aaagtgcacaggatttcagtgaa

SEQ ID NO: 20 - RNA, 23 nt, synthetic construct
  source 1..23: mol_type = other RNA
tacaagccagattagaaagtgca

SEQ ID NO: 21 - RNA, 23 nt, synthetic construct
  source 1..23: mol_type = other RNA
aaggtaaaaaacaagcaaccggg

SEQ ID NO: 22 - RNA, 23 nt, synthetic construct
  source 1..23: mol_type = other RNA
tctttgagattctttgggccatt

SEQ ID NO: 23 - RNA, 23 nt, synthetic construct
  source 1..23: mol_type = other RNA
tagacaaagatttctttgagatt

SEQ ID NO: 24 - RNA, 23 nt, synthetic construct
  source 1..23: mol_type = other RNA
tgttttaaggagacaaagatttc

SEQ ID NO: 25 - RNA, 23 nt, synthetic construct
  source 1..23: mol_type = other RNA
atgttttaaggagacaaagattt

SEQ ID NO: 26 - RNA, 23 nt, synthetic construct
  source 1..23: mol_type = other RNA
tctcagatgttttaaggagacaa

SEQ ID NO: 27 - RNA, 23 nt, synthetic construct
  source 1..23: mol_type = other RNA
tctttttaatgcgtgtactgggc

SEQ ID NO: 28 - RNA, 23 nt, synthetic construct
  source 1..23: mol_type = other RNA
tgaaaccagaaagagctttgctc

SEQ ID NO: 29 - RNA, 23 nt, synthetic construct
  source 1..23: mol_type = other RNA
actgaaaccagaaagagctttgc

SEQ ID NO: 30 - RNA, 23 nt, synthetic construct
  source 1..23: mol_type = other RNA
tgtaaaatgaagaatggcagaac

SEQ ID NO: 31 - RNA, 23 nt, synthetic construct
  source 1..23: mol_type = other RNA
taagaaatcagtgtcatggtaaa

SEQ ID NO: 32 - RNA, 23 nt, synthetic construct
  source 1..23: mol_type = other RNA
atatccagttcttctcccaagaa

SEQ ID NO: 33 - RNA, 23 nt, synthetic construct
  source 1..23: mol_type = other RNA
agcaacaatatccagttcttctc

SEQ ID NO: 34 - RNA, 23 nt, synthetic construct
  source 1..23: mol_type = other RNA
tattggtgcacagtttctggcag

SEQ ID NO: 35 - RNA, 23 nt, synthetic construct
  source 1..23: mol_type = other RNA
ataggtaaaaaactggcagcgga

SEQ ID NO: 36 - RNA, 23 nt, synthetic construct
  source 1..23: mol_type = other RNA
ttgaagaaagctttaagtaacac

SEQ ID NO: 37 - RNA, 23 nt, synthetic construct
  source 1..23: mol_type = other RNA
ttaatgtgtatccagagatgcct

SEQ ID NO: 38 - RNA, 23 nt, synthetic construct
  source 1..23: mol_type = other RNA
tgattttggtggtacactcatta

SEQ ID NO: 39 - RNA, 23 nt, synthetic construct
  source 1..23: mol_type = other RNA
ttgattttggtggtacactcatt

SEQ ID NO: 40 - RNA, 23 nt, synthetic construct
  source 1..23: mol_type = other RNA
tttccaatgatggagcctccaca

SEQ ID NO: 41 - RNA, 23 nt, synthetic construct
  source 1..23: mol_type = other RNA
aaaatcttaggtgactctacccc

SEQ ID NO: 42 - RNA, 23 nt, synthetic construct
  source 1..23: mol_type = other RNA
tcagattgatttaaaatgccact

SEQ ID NO: 43 - RNA, 23 nt, synthetic construct
  source 1..23: mol_type = other RNA
taacaaggcaatatcatacccgc

SEQ ID NO: 44 - RNA, 23 nt, synthetic construct
  source 1..23: mol_type = other RNA
ttgtaattcactgtggtttccag

SEQ ID NO: 45 - RNA, 23 nt, synthetic construct
  source 1..23: mol_type = other RNA
agaatctgtgtaattcactgtgg

SEQ ID NO: 46 - RNA, 23 nt, synthetic construct
  source 1..23: mol_type = other RNA
ttgagaatctgtgtaattcactg

SEQ ID NO: 47 - RNA, 23 nt, synthetic construct
  source 1..23: mol_type = other RNA
tatcttggctttctggagagtat

SEQ ID NO: 48 - RNA, 23 nt, synthetic construct
  source 1..23: mol_type = other RNA
actaagggtatcttggctttctg

SEQ ID NO: 49 - RNA, 23 nt, synthetic construct
  source 1..23: mol_type = other RNA
tatgtcctctgtatctcttctgg

SEQ ID NO: 50 - RNA, 23 nt, synthetic construct
  source 1..23: mol_type = other RNA
tatgggttattttatgtcctctg

SEQ ID NO: 51 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
agtcacacattcaccagaaactg

SEQ ID NO: 52 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
aatgtcccctccttcaaagcagg

SEQ ID NO: 53 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
attcactcttggcagtgtttctg

SEQ ID NO: 54 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tgaaagaatacccagaaatcgct

SEQ ID NO: 55 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
ttgcttgaaagaatacccagaaa

SEQ ID NO: 56 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
attgcttgaaagaatacccagaa

SEQ ID NO: 57 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tattgcttgaaagaatacccaga

SEQ ID NO: 58 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tatttggtgtgagcattgcttga

SEQ ID NO: 59 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
atgtctttgttgcaagcgcttat

SEQ ID NO: 60 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
aatgtctttgttgcaagcgctta

SEQ ID NO: 61 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
ataaatgtctttgttgcaagcgc

SEQ ID NO: 62 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
acataaatgtctttgttgcaagc

SEQ ID NO: 63 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
ttatagtttatgcccttcatgtc

SEQ ID NO: 64 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tttcttggcattcttgagcactc

SEQ ID NO: 65 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
acaaatgttacgatgctccaggc

SEQ ID NO: 66 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tagtagacaaatgttacgatgct

SEQ ID NO: 67 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tttcagtagacaaatgttacgat

SEQ ID NO: 68 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tatttcagtgaaaatccagacac

SEQ ID NO: 69 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
aaagtgcacaggatttcagtgaa

SEQ ID NO: 70 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tacaagccagattagaaagtgca

SEQ ID NO: 71 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
aaggtaaaaaacaagcaaccggg

SEQ ID NO: 72 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tctttgagattctttgggccatt

SEQ ID NO: 73 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tagacaaagatttctttgagatt

SEQ ID NO: 74 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tgttttaaggagacaaagatttc

SEQ ID NO: 75 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
atgttttaaggagacaaagattt

SEQ ID NO: 76 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tctcagatgttttaaggagacaa

SEQ ID NO: 77 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tctttttaatgcgtgtactgggc

SEQ ID NO: 78 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tgaaaccagaaagagctttgctc

SEQ ID NO: 79 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
actgaaaccagaaagagctttgc

SEQ ID NO: 80 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tgtaaaatgaagaatggcagaac

SEQ ID NO: 81 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
taagaaatcagtgtcatggtaaa

SEQ ID NO: 82 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
atatccagttcttctcccaagaa

SEQ ID NO: 83 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
agcaacaatatccagttcttctc

SEQ ID NO: 84 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tattggtgcacagtttctggcag

SEQ ID NO: 85 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
ataggtaaaaaactggcagcgga

SEQ ID NO: 86 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
ttgaagaaagctttaagtaacac

SEQ ID NO: 87 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
ttaatgtgtatccagagatgcct

SEQ ID NO: 88 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tgattttggtggtacactcatta

SEQ ID NO: 89 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
ttgattttggtggtacactcatt

SEQ ID NO: 90 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tttccaatgatggagcctccaca

SEQ ID NO: 91 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
aaaatcttaggtgactctacccc

SEQ ID NO: 92 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tcagattgatttaaaatgccact

SEQ ID NO: 93 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
taacaaggcaatatcatacccgc

SEQ ID NO: 94 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
ttgtaattcactgtggtttccag

SEQ ID NO: 95 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
agaatctgtgtaattcactgtgg

SEQ ID NO: 96 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
ttgagaatctgtgtaattcactg

SEQ ID NO: 97 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tatcttggctttctggagagtat

SEQ ID NO: 98 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
actaagggtatcttggctttctg

SEQ ID NO: 99 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tatgtcctctgtatctcttctgg

SEQ ID NO: 100 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tatgggttattttatgtcctctg

SEQ ID NO: 101 - RNA, 21 nt, synthetic construct
  source 1..21: mol_type = other RNA
gtttctggtgaatgtgtgact

SEQ ID NO: 102 - RNA, 21 nt, synthetic construct
  source 1..21: mol_type = other RNA
tgctttgaaggaggggacatt

SEQ ID NO: 103 - RNA, 21 nt, synthetic construct
  source 1..21: mol_type = other RNA
gaaacactgccaagagtgaat

SEQ ID NO: 104 - RNA, 21 nt, synthetic construct
  source 1..21: mol_type = other RNA
cgatttctgggtattctttca

SEQ ID NO: 105 - RNA, 21 nt, synthetic construct
  source 1..21: mol_type = other RNA
tctgggtattctttcaagcaa

SEQ ID NO: 106 - RNA, 21 nt, synthetic construct
  source 1..21: mol_type = other RNA
ctgggtattctttcaagcaat

SEQ ID NO: 107 - RNA, 21 nt, synthetic construct
  source 1..21: mol_type = other RNA
tgggtattctttcaagcaata

SEQ ID NO: 108 - RNA, 21 nt, synthetic construct
  source 1..21: mol_type = other RNA
aagcaatgctcacaccaaata

SEQ ID NO: 109 - RNA, 21 nt, synthetic construct
  source 1..21: mol_type = other RNA
aagcgcttgcaacaaagacat

SEQ ID NO: 110 - RNA, 21 nt, synthetic construct
  source 1..21: mol_type = other RNA
agcgcttgcaacaaagacatt

SEQ ID NO: 111 - RNA, 21 nt, synthetic construct
  source 1..21: mol_type = other RNA
gcttgcaacaaagacatttat

SEQ ID NO: 112 - RNA, 21 nt, synthetic construct
  source 1..21: mol_type = other RNA
ttgcaacaaagacatttatgt

SEQ ID NO: 113 - RNA, 21 nt, synthetic construct
  source 1..21: mol_type = other RNA
catgaagggcataaactataa

SEQ ID NO: 114 - RNA, 21 nt, synthetic construct
  source 1..21: mol_type = other RNA
gtgctcaagaatgccaagaaa

SEQ ID NO: 115 - RNA, 21 nt, synthetic construct
  source 1..21: mol_type = other RNA
ctggagcatcgtaacatttgt

SEQ ID NO: 116 - RNA, 21 nt, synthetic construct
  source 1..21: mol_type = other RNA
catcgtaacatttgtctacta

SEQ ID NO: 117 - RNA, 21 nt, synthetic construct
  source 1..21: mol_type = other RNA
cgtaacatttgtctactgaaa

SEQ ID NO: 118 - RNA, 21 nt, synthetic construct
  source 1..21: mol_type = other RNA
gtctggattttcactgaaata

SEQ ID NO: 119 - RNA, 21 nt, synthetic construct
  source 1..21: mol_type = other RNA
cactgaaatcctgtgcacttt

SEQ ID NO: 120 - RNA, 21 nt, synthetic construct
  source 1..21: mol_type = other RNA
cactttctaatctggcttgta

SEQ ID NO: 121 - RNA, 21 nt, synthetic construct
  source 1..21: mol_type = other RNA
cggttgcttgttttttacctt

SEQ ID NO: 122 - RNA, 21 nt, synthetic construct
  source 1..21: mol_type = other RNA
tggcccaaagaatctcaaaga

SEQ ID NO: 123 - RNA, 21 nt, synthetic construct
  source 1..21: mol_type = other RNA
tctcaaagaaatctttgtcta

SEQ ID NO: 124 - RNA, 21 nt, synthetic construct
  source 1..21: mol_type = other RNA
aatctttgtctccttaaaaca

SEQ ID NO: 125 - RNA, 21 nt, synthetic construct
  source 1..21: mol_type = other RNA
atctttgtctccttaaaacat

SEQ ID NO: 126 - RNA, 21 nt, synthetic construct
  source 1..21: mol_type = other RNA
gtctccttaaaacatctgaga

SEQ ID NO: 127 - RNA, 21 nt, synthetic construct
  source 1..21: mol_type = other RNA
ccagtacacgcattaaaaaga

SEQ ID NO: 128 - RNA, 21 nt, synthetic construct
  source 1..21: mol_type = other RNA
gcaaagctctttctggtttca

SEQ ID NO: 129 - RNA, 21 nt, synthetic construct
  source 1..21: mol_type = other RNA
aaagctctttctggtttcagt

SEQ ID NO: 130 - RNA, 21 nt, synthetic construct
  source 1..21: mol_type = other RNA
tctgccattcttcattttaca

SEQ ID NO: 131 - RNA, 21 nt, synthetic construct
  source 1..21: mol_type = other RNA
taccatgacactgatttctta

SEQ ID NO: 132 - RNA, 21 nt, synthetic construct
  source 1..21: mol_type = other RNA
cttgggagaagaactggatat

SEQ ID NO: 133 - RNA, 21 nt, synthetic construct
  source 1..21: mol_type = other RNA
gaagaactggatattgttgct

SEQ ID NO: 134 - RNA, 21 nt, synthetic construct
  source 1..21: mol_type = other RNA
gccagaaactgtgcaccaata

SEQ ID NO: 135 - RNA, 21 nt, synthetic construct
  source 1..21: mol_type = other RNA
cgctgccagttttttacctat

SEQ ID NO: 136 - RNA, 21 nt, synthetic construct
  source 1..21: mol_type = other RNA
gttacttaaagctttcttcaa

SEQ ID NO: 137 - RNA, 21 nt, synthetic construct
  source 1..21: mol_type = other RNA
gcatctctggatacacattaa

SEQ ID NO: 138 - RNA, 21 nt, synthetic construct
  source 1..21: mol_type = other RNA
atgagtgtaccaccaaaatca

SEQ ID NO: 139 - RNA, 21 nt, synthetic construct
  source 1..21: mol_type = other RNA
tgagtgtaccaccaaaatcaa

SEQ ID NO: 140 - RNA, 21 nt, synthetic construct
  source 1..21: mol_type = other RNA
tggaggctccatcattggaaa

SEQ ID NO: 141 - RNA, 21 nt, synthetic construct
  source 1..21: mol_type = other RNA
ggtagagtcacctaagatttt

SEQ ID NO: 142 - RNA, 21 nt, synthetic construct
  source 1..21: mol_type = other RNA
tggcattttaaatcaatctga

SEQ ID NO: 143 - RNA, 21 nt, synthetic construct
  source 1..21: mol_type = other RNA
gggtatgatattgccttgtta

SEQ ID NO: 144 - RNA, 21 nt, synthetic construct
  source 1..21: mol_type = other RNA
ggaaaccacagtgaattacaa

SEQ ID NO: 145 - RNA, 21 nt, synthetic construct
  source 1..21: mol_type = other RNA
acagtgaattacacagattct

SEQ ID NO: 146 - RNA, 21 nt, synthetic construct
  source 1..21: mol_type = other RNA
gtgaattacacagattctcaa

SEQ ID NO: 147 - RNA, 21 nt, synthetic construct
  source 1..21: mol_type = other RNA
actctccagaaagccaagata

SEQ ID NO: 148 - RNA, 21 nt, synthetic construct
  source 1..21: mol_type = other RNA
gaaagccaagatacccttagt

SEQ ID NO: 149 - RNA, 21 nt, synthetic construct
  source 1..21: mol_type = other RNA
agaagagatacagaggacata

SEQ ID NO: 150 - RNA, 21 nt, synthetic construct
  source 1..21: mol_type = other RNA
gaggacataaaataacccata

SEQ ID NO: 151 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
gtttctggtgaatgtgtgact

SEQ ID NO: 152 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
tgctttgaaggaggggacatt

SEQ ID NO: 153 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
gaaacactgccaagagtgaat

SEQ ID NO: 154 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
cgatttctgggtattctttca

SEQ ID NO: 155 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
tctgggtattctttcaagcaa

SEQ ID NO: 156 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
ctgggtattctttcaagcaat

SEQ ID NO: 157 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
tgggtattctttcaagcaata

SEQ ID NO: 158 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
aagcaatgctcacaccaaata

SEQ ID NO: 159 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
aagcgcttgcaacaaagacat

SEQ ID NO: 160 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
agcgcttgcaacaaagacatt

SEQ ID NO: 161 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
gcttgcaacaaagacatttat

SEQ ID NO: 162 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
ttgcaacaaagacatttatgt

SEQ ID NO: 163 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
catgaagggcataaactataa

SEQ ID NO: 164 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
gtgctcaagaatgccaagaaa

SEQ ID NO: 165 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
ctggagcatcgtaacatttgt

SEQ ID NO: 166 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
catcgtaacatttgtctacta

SEQ ID NO: 167 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
cgtaacatttgtctactgaaa

SEQ ID NO: 168 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
gtctggattttcactgaaata

SEQ ID NO: 169 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
cactgaaatcctgtgcacttt

SEQ ID NO: 170 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
cactttctaatctggcttgta

SEQ ID NO: 171 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
cggttgcttgttttttacctt

SEQ ID NO: 172 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
tggcccaaagaatctcaaaga

SEQ ID NO: 173 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
tctcaaagaaatctttgtcta

SEQ ID NO: 174 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
aatctttgtctccttaaaaca

SEQ ID NO: 175 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
atctttgtctccttaaaacat

SEQ ID NO: 176 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
gtctccttaaaacatctgaga

SEQ ID NO: 177 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
ccagtacacgcattaaaaaga

SEQ ID NO: 178 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
gcaaagctctttctggtttca

SEQ ID NO: 179 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
aaagctctttctggtttcagt

SEQ ID NO: 180 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
tctgccattcttcattttaca

SEQ ID NO: 181 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
taccatgacactgatttctta

SEQ ID NO: 182 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
cttgggagaagaactggatat

SEQ ID NO: 183 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
gaagaactggatattgttgct

SEQ ID NO: 184 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
gccagaaactgtgcaccaata

SEQ ID NO: 185 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
cgctgccagttttttacctat

SEQ ID NO: 186 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
gttacttaaagctttcttcaa

SEQ ID NO: 187 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
gcatctctggatacacattaa

SEQ ID NO: 188 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
atgagtgtaccaccaaaatca

SEQ ID NO: 189 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
tgagtgtaccaccaaaatcaa

SEQ ID NO: 190 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
tggaggctccatcattggaaa

SEQ ID NO: 191 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
ggtagagtcacctaagatttt

SEQ ID NO: 192 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
tggcattttaaatcaatctga

SEQ ID NO: 193 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
gggtatgatattgccttgtta

SEQ ID NO: 194 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
ggaaaccacagtgaattacaa

SEQ ID NO: 195 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
acagtgaattacacagattct

SEQ ID NO: 196 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
gtgaattacacagattctcaa

SEQ ID NO: 197 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
actctccagaaagccaagata

SEQ ID NO: 198 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
gaaagccaagatacccttagt

SEQ ID NO: 199 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
agaagagatacagaggacata

SEQ ID NO: 200 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
gaggacataaaataacccata

SEQ ID NO: 201 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..6: mod_base = OTHER; note = 2'-O-Methyl ribonucleotide
  modified_base 7: mod_base = OTHER; note = 2'-fluoro ribonucleotide
  modified_base 8..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tgaaagaatacccagaaatcgct

SEQ ID NO: 202 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..6: mod_base = OTHER; note = 2'-O-Methyl ribonucleotide
  modified_base 7: mod_base = OTHER; note = 2'-fluoro ribonucleotide
  modified_base 8..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
attgcttgaaagaatacccagaa

SEQ ID NO: 203 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..6: mod_base = OTHER; note = 2'-O-Methyl ribonucleotide
  modified_base 7: mod_base = OTHER; note = 2'-fluoro ribonucleotide
  modified_base 8..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tattgcttgaaagaatacccaga

SEQ ID NO: 204 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..6: mod_base = OTHER; note = 2'-O-Methyl ribonucleotide
  modified_base 7: mod_base = OTHER; note = 2'-fluoro ribonucleotide
  modified_base 8..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
atgtctttgttgcaagcgcttat

SEQ ID NO: 205 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..6: mod_base = OTHER; note = 2'-O-Methyl ribonucleotide
  modified_base 7: mod_base = OTHER; note = 2'-fluoro ribonucleotide
  modified_base 8..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
aatgtctttgttgcaagcgctta

SEQ ID NO: 206 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..6: mod_base = OTHER; note = 2'-O-Methyl ribonucleotide
  modified_base 7: mod_base = OTHER; note = 2'-fluoro ribonucleotide
  modified_base 8..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
ataaatgtctttgttgcaagcgc

SEQ ID NO: 207 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..6: mod_base = OTHER; note = 2'-O-Methyl ribonucleotide
  modified_base 7: mod_base = OTHER; note = 2'-fluoro ribonucleotide
  modified_base 8..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
ttatagtttatgcccttcatgtc

SEQ ID NO: 208 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..6: mod_base = OTHER; note = 2'-O-Methyl ribonucleotide
  modified_base 7: mod_base = OTHER; note = 2'-fluoro ribonucleotide
  modified_base 8..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tttcttggcattcttgagcactc

SEQ ID NO: 209 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..6: mod_base = OTHER; note = 2'-O-Methyl ribonucleotide
  modified_base 7: mod_base = OTHER; note = 2'-fluoro ribonucleotide
  modified_base 8..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
ataggtaaaaaactggcagcgga

SEQ ID NO: 210 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..6: mod_base = OTHER; note = 2'-O-Methyl ribonucleotide
  modified_base 7: mod_base = OTHER; note = 2'-fluoro ribonucleotide
  modified_base 8..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
tatgggttattttatgtcctctg

SEQ ID NO: 211 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 17..18: mod_base = OTHER; note = phosphorothioate linkage
  modified_base 21..23: mod_base = OTHER; note = phosphorothioate linkage
ataaatgtctttgttgcaagcgc

SEQ ID NO: 212 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 14..15: mod_base = OTHER; note = phosphorothioate linkage
  modified_base 21..23: mod_base = OTHER; note = phosphorothioate linkage
ataaatgtctttgttgcaagcgc

SEQ ID NO: 213 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 19..20: mod_base = OTHER; note = phosphorothioate linkage
  modified_base 21..23: mod_base = OTHER; note = phosphorothioate linkage
ataaatgtctttgttgcaagcgc

SEQ ID NO: 214 - DNA, 23 bp, synthetic construct
  source 1..23: mol_type = other DNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 22..23: mod_base = OTHER; note = 3' inverted thymidine
  modified_base 1..3: mod_base = OTHER; note = phosphorothioate linkage
  misc_feature 1..21: note = RNA
  misc_feature 22..23: note = DNA
gcttgcaacaaagacatttattt

SEQ ID NO: 215 - RNA, 23 nt, synthetic construct
  source 1..23: mol_type = other RNA
  modified_base 1: mod_base = OTHER; note = inosine
ntaaatgtctttgttgcaagcgc

SEQ ID NO: 216 - RNA, 23 nt, synthetic construct
  source 1..23: mol_type = other RNA
ttaaatgtctttgttgcaagcgc

SEQ ID NO: 217 - RNA, 23 nt, synthetic construct
  source 1..23: mol_type = other RNA
  modified_base 1: mod_base = OTHER; note = 2'-O-methylinosine
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
ntaaatgtctttgttgcaagcgc

SEQ ID NO: 218 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..6: mod_base = OTHER; note = 2'-O-Methyl ribonucleotide
  modified_base 7: mod_base = OTHER; note = 2'-fluoro ribonucleotide
  modified_base 8..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
ttaaatgtctttgttgcaagcgc

SEQ ID NO: 219 - RNA, 23 nt, synthetic construct
  source 1..23: mol_type = other RNA
  modified_base 1: mod_base = OTHER; note = 2'-O-methyl-8-bromo-adenosine
  modified_base 2: mod_base = OTHER; note = 2'-fluoro ribonucleotide
  modified_base 3..6: mod_base = OTHER; note = 2'-O-Methyl ribonucleotide
  modified_base 7: mod_base = OTHER; note = 2'-fluoro ribonucleotide
  modified_base 8..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
ataaatgtctttgttgcaagcgc

SEQ ID NO: 220 - RNA, 21 nt, synthetic construct
  source 1..21: mol_type = other RNA
gcttgcaacaaagacatttaa

SEQ ID NO: 221 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
gcttgcaacaaagacatttaa

SEQ ID NO: 222 - RNA, 21 nt, synthetic construct
  source 1..21: mol_type = other RNA
  modified_base 1..6: mod_base = OTHER; note = 2'-O-Methyl ribonucleotide
  modified_base 7: mod_base = OTHER; note = 2'-fluoro ribonucleotide
  modified_base 8: mod_base = OTHER; note = 2'-O-Methyl ribonucleotide
  modified_base 9: mod_base = OTHER; note = 2'-fluoro ribonucleotide
  modified_base 10: mod_base = OTHER; note = 2'-O-Methyl ribonucleotide
  modified_base 11: mod_base = OTHER; note = 2'-fluoro ribonucleotide
  modified_base 12..21: mod_base = OTHER; note = 2'-O-Methyl ribonucleotide
  modified_base 1..3: mod_base = OTHER; note = phosphorothioate linkage
gcttgcaacaaagacatttaa

SEQ ID NO: 223 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 21..23: mod_base = OTHER; note = phosphorothioate linkage
ataaatgtctttgttgcaagcgc

SEQ ID NO: 224 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..4: mod_base = OTHER; note = phosphorothioate linkage
  modified_base 21..23: mod_base = OTHER; note = phosphorothioate linkage
ataaatgtctttgttgcaagcgc

SEQ ID NO: 225 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 5..6: mod_base = OTHER; note = phosphorothioate linkage
  modified_base 21..23: mod_base = OTHER; note = phosphorothioate linkage
ataaatgtctttgttgcaagcgc

SEQ ID NO: 226 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 5..6: mod_base = OTHER; note = phosphorothioate linkage
  modified_base 17..18: mod_base = OTHER; note = phosphorothioate linkage
  modified_base 21..23: mod_base = OTHER; note = phosphorothioate linkage
ataaatgtctttgttgcaagcgc

SEQ ID NO: 227 - RNA, 23 nt, synthetic construct
  source 1..23: mol_type = other RNA
  modified_base 1: mod_base = OTHER; note = 2'-O-Methyl ribonucleotide
  modified_base 2: mod_base = OTHER; note = 2'-fluoro ribonucleotide
  modified_base 3..11: mod_base = OTHER; note = 2'-O-Methyl ribonucleotide
  modified_base 12: mod_base = OTHER; note = 2'-fluoro ribonucleotide
  modified_base 13: mod_base = OTHER; note = 2'-O-Methyl ribonucleotide
  modified_base 14: mod_base = OTHER; note = 2'-fluoro ribonucleotide
  modified_base 15: mod_base = OTHER; note = 2'-O-Methyl ribonucleotide
  modified_base 16: mod_base = OTHER; note = 2'-fluoro ribonucleotide
  modified_base 17..23: mod_base = OTHER; note = 2'-O-Methyl ribonucleotide
  modified_base 1..3: mod_base = OTHER; note = phosphorothioate linkage
  modified_base 5..6: mod_base = OTHER; note = phosphorothioate linkage
  modified_base 14..15: mod_base = OTHER; note = phosphorothioate linkage
  modified_base 21..23: mod_base = OTHER; note = phosphorothioate linkage
ataaatgtctttgttgcaagcgc

SEQ ID NO: 228 - protein, 5 aa, synthetic construct
YIGSR

SEQ ID NO: 229 - protein, 4 aa, synthetic construct
  SITE 1: note = N-formyl-methionyl
  SITE 2: note = leucyl
MLFK